(19) United States
(12) Patent Application Publication
(10) Pub. No.: US 2023/0065628 A1
(43) Pub. Date: Mar. 2, 2023

(54) Droplet Formation and Particle Morphology

(71) Applicant: Elektrofi, Inc., Boston, MA (US)

(72) Inventors: Jason G. Auer, Weymouth, MA (US); Paul Brown, Boston, MA (US); Chase Spenser Coffman, Newton, MA (US); James W. Ivey, Reading, MA (US); Lisa Liu, Somerville, MA (US); Shankul Vartak, Cambridge, MA (US)

(21) Appl. No.: 17/759,592

(22) PCT Filed: Feb. 19, 2021
(86) PCT No.: PCT/US2021/018806

§ 371 Date: Jul. 27, 2022

Related U.S. Application Data
(60) Provisional application No. 62/978,641, filed on Feb. 19, 2020.

(51) Int. Cl. A61K 9/16; C07K 16/28

(52) U.S. Cl. CPC A61K 9/1682; A61K 9/1617; C07K 16/2887

(57) ABSTRACT

The present disclosure relates to compositions and methods that enable the formation of pharmaceutically relevant particles that can be used for therapy. In particular, the methods disclosed herein allow the controlled formation of circular particles having low internal void spaces comprising bioactive therapeutic biologies.

### RELATED APPLICATION(S)

This application claims the benefit of U.S. Provisional Application No. 62/978,641, filed Feb. 19, 2020. The entire teachings of the above application are incorporated herein by reference.

### TECHNICAL FIELD

The present disclosure relates to methods that enable the formation of pharmaceutically relevant particles that can be used for therapy. In particular, the methods disclosed herein allow the formation of droplets that produce circular particles comprising therapeutic biologics having low internal void spaces.

### BACKGROUND

Materials science and the application of nanotechnology calls for more efficient, reproducible and innovative technologies to synthesize novel functional particles. Recent advances in synthesis and the controlled assembly of bioactive particles have enabled their applications for use in therapy. Current efforts have been directed to developing new synthetic approaches for non-circular microparticles that often exhibit physical properties unobtainable by simply tuning the size and form of the particles. However, the application of these techniques to circular particles have been limited due to the lack of sufficient control over size uniformity, shape selectivity, surface functionality and skeletal density of the particles which are often difficult to obtain. Therefore, a highly robust and controlled method for particle preparation is needed.

### SUMMARY

Provided herein are methods allowing the formation of droplets comprising a therapeutic biologic that produce circular particles having low internal void spaces.

In one aspect, the disclosure provides a method of forming particles, the method comprising:

a) providing a first liquid comprising a therapeutic biologic and a solvent;

b) contacting the first liquid with a second liquid, thereby forming liquid droplets comprising the therapeutic biologic;

c) contacting the liquid droplets with a third liquid, thereby allowing the liquid droplets to dry; and

d) removing the first liquid, second liquid, and third liquid, thereby forming particles comprising a therapeutic biologic, wherein the particles comprise less than about 10% internal void spaces and the circularity of the particles is from about 0.80 to about 1.00 after removing the first liquid, second liquid, and third liquid.

The present disclosure also provides herein, a method of controlling the morphology of particles.

In another aspect, the disclosure provides a method of controlling the morphology of particles, the method comprising:

a) providing a first liquid comprising a therapeutic biologic and a solvent;

b) contacting the first liquid with a second liquid, thereby forming liquid droplets comprising the therapeutic biologic;

c) contacting the liquid droplets with a third liquid, thereby forming a mixture, wherein the Peclet number of the mixture determines the morphology of the particles;

d) allowing the liquid droplets to dry; and

e) removing the first liquid, second liquid, and third liquid,

thereby forming particles comprising a therapeutic biologic, wherein the particles comprise less than about 10% internal void spaces and the circularity of the particles is from about 0.80 to about 1.00 after removing the first liquid, second liquid, and third liquid.

The present methods may be useful for the formation of pharmaceutically relevant particles that can be used for therapy. In preferred embodiments, the methods disclosed herein may allow the formation of droplets comprising a therapeutic biologic that produce circular particles having low internal void spaces.

### BRIEF DESCRIPTION OF THE DRAWINGS

The foregoing will be apparent from the following more particular description of example embodiments, as illustrated in the accompanying drawings in which like reference characters, refer to the same parts throughout the different views. The drawings are not necessarily to scale, emphasis instead being placed upon illustrating embodiments.

FIG. 1 shows images of a typical experiment of a dispersed phase added to a continuous phase to form droplets of the disclosure.

FIG. 2 shows an image of bovine serum albumin (BSA) particles produced using an impinging jet mixer to form droplets of the disclosure.

FIG. 3 shows a diagram of an exemplary impinging jet mixer used to form particles comprising a therapeutic biologic through methods of the disclosure.

FIG. 4 shows an image of bovine serum albumin (BSA) particles produced using an impinging jet mixer to form droplets of the disclosure.

FIG. 5 shows a diagram of an exemplary impinging jet mixer used to form particles comprising a therapeutic biologic through methods of the disclosure.

FIG. 6 shows an image of monoclonal antibody (mAb) particles produced using an impinging jet mixer to form droplets of the disclosure.

FIG. 7 shows an image of bovine serum albumin (BSA) particles produced using an impinging jet mixer to form droplets of the disclosure.

FIG. 8 shows a diagram of an exemplary membrane emulsification system used to form particles comprising a therapeutic biologic through methods of the disclosure.

FIG. 9 shows an image of BIgG particles produced using membrane emulsification to form droplets of the disclosure.

FIG. 10 shows an image of BIgG particles produced using membrane emulsification to form droplets of the disclosure.

FIG. 11 shows an image of BSA particles produced using membrane emulsification to form droplets of the disclosure.

FIG. 12 shows an image of monoclonal antibody particles produced using membrane emulsification to form droplets of the disclosure.

FIG. 13 shows an image of HIgG particles produced using membrane emulsification to form droplets of the disclosure.

FIG. 14 shows an image of Human IgG particles produced using a rotor-stator mixer to form droplets of the disclosure.

FIG. 15 shows a diagram of an exemplary micromixer system used to form particles comprising a therapeutic biologic through methods of the disclosure.

FIG. 16 shows a graph of typical average droplet sizes dispersed in the continuous phase versus CP flow rate according to methods of the disclosure.

FIG. 17 shows an image of a human IgG particle produced using a micromixer system to form droplets of the disclosure.

### DETAILED DESCRIPTION

Particles have been produced using various techniques. For example, the generation of particles can be accomplished by producing a droplet of a liquid comprising an active agent dissolved in a solvent. The solvent can then be extracted from the droplets by depositing the droplets into a liquid in which the solvent, but not the active agent is soluble leaving behind a solid particle. Isolation of the particles occur following removal of the liquids. However, the application of these techniques to form functional circular particles have been limited due to the lack of sufficient control over size uniformity, shape selectivity, surface functionality and skeletal density of the particles which are often difficult to obtain. The present disclosure seeks to mitigate the control issues that are associated with forming functional particles by providing a robust and controlled method for particle preparation.

The present disclosure generally relates to methods of forming particles, the method comprising: a) providing a first liquid comprising a therapeutic biologic and a solvent; b) contacting the first liquid with a second liquid, thereby forming liquid droplets comprising the therapeutic biologic; c) contacting the liquid droplets with a third liquid, thereby allowing the liquid droplets to dry; and d) removing the first liquid, second liquid, and third liquid, thereby forming particles comprising a therapeutic biologic, wherein the particles comprise less than about 10% internal void spaces and the circularity of the particles is from about 0.80 to about 1.00 after removing the first liquid, second liquid, and third liquid.

In certain aspects, the disclosure generally relates to a method of controlling the morphology of particles, the method comprising: a) providing a first liquid comprising a therapeutic biologic and a solvent; b) contacting the first liquid with a second liquid, thereby forming liquid droplets comprising the therapeutic biologic; c) contacting the liquid droplets with a third liquid, thereby forming a mixture, wherein the Peclet number of the mixture determines the morphology of the particles; d) allowing the liquid droplets to dry; and e) removing the first liquid, second liquid, and third liquid, thereby forming particles comprising a therapeutic biologic, wherein the particles comprise less than about 10% internal void spaces and the circularity of the particles is from about 0.80 to about 1.00 after removing the first liquid, second liquid, and third liquid.

As described herein, the disclosure provides methods for the preparation of particles comprising a therapeutic biologic, e.g., an antibody, bovine serum albumin (BSA), or human serum albumin (HSA). In preferred embodiments, the therapeutic biologic is an antibody, bovine serum albumin (BSA), or human serum albumin (HSA). The generation of particles can be accomplished by providing a first liquid comprising a therapeutic biologic and a solvent. The first liquid can then be contacted with a second liquid, thereby forming liquid droplets comprising the therapeutic biologic. The solvent can then be removed from the droplets by contacting the droplets to a third liquid in which the solvent, but not the therapeutic biologic, is soluble leaving behind a solid particle. Isolation of the particles occur following removal of the solvent and liquids. The process of forming the droplets as described herein, can significantly alter the structure and morphology of the particles and may enhance the stability of the therapeutic biologic. These particles may be used to generate stabilized pharmaceutical compositions, pharmaceutical suspension formulations, pharmaceutical powder formulations (e.g., inhalable powders, injectable powders), creams or other topical pastes, nutraceuticals, or cosmetics. The term “pharmaceutical composition” as used herein, denotes a composition in which a therapeutic biologic retains, or partially retains, its intended biological activity or functional form, and in which only pharmaceutically acceptable components are included.

It will be readily understood that the aspects and embodiments, as generally described herein, are exemplary. The following more detailed description of various aspects and embodiments are not intended to limit the scope of the present disclosure, but is merely representative of various aspects and embodiments. Moreover, the methods disclosed herein may be changed by those skilled in the art without departing from the scope of the present disclosure. Unless defined otherwise, all technical and scientific terms used herein have the same meaning as is commonly understood by one of skill in the art to which this disclosure belongs. All publications and patents referred to herein are incorporated by reference.

### Definitions

For purposes of the present disclosure, the following definitions will be used unless expressly stated otherwise:

The terms “a”, “an”, “the” and similar referents used in the context of describing the present disclosure are to be construed to cover both the singular and the plural, unless otherwise indicated herein or clearly contradicted by context. All methods described herein, can be performed in any suitable order unless otherwise indicated herein or otherwise clearly contradicted by context. The use of any and all examples, or exemplary language (e.g., “such as”) provided herein, is intended merely to better illuminate the present disclosure and does not pose a limitation on the scope of the disclosure otherwise claimed. No language in the present specification should be construed as indicating any unclaimed element is essential to the practice of the disclosure.

The term “about” in relation to a given numerical value, such as for temperature and period of time, is meant to include numerical values within 10% of the specified value.

As used herein, an “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, iso-butyl, sec-butyl, tert-butyl, n-pentyl, tert-pentyl, neo-pentyl, iso-pentyl, sec-pentyl, 3-pentyl, sec-iso-pentyl, active-pentyl, hexyl, heptyl, octyl, ethylhexyl, and the like. A C₁₈ straight chained or branched alkyl group is also referred to as a “lower alkyl” group. An alkyl group with two open valences is sometimes referred to as an alkylene group, such as methylene, ethylene, propylene and the like. Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, an alkyl, a halogen, a hydroxyl, a carbonyl (such as a carboxyl, and alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamide, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN and the like. In other embodiments, the term “alkyl” can mean “cycloalkyl” which refers to a non-aromatic carbocyclic ring having 3 to 10 carbon ring atoms, which are carbon atoms bound together to form the ring. The ring may be saturated or have one or more carbon-carbon double bonds. Examples of cycloalkyl include, but not limited to cyclopropyl, cyclobutyl, cyclopentyl, cyclopentenyl, cyclohexyl, cyclohexenyl, and cycloheptyl, as well as bridged and caged saturated ring groups such as norbornyl and adamantyl. As described herein, organic solvents include, but are not limited to aliphatic hydrocarbon solvents, aromatic hydrocarbon solvents, alcohols or alkylalcohols, alkylethers, sulfoxides, alkylketones, alkylacetates, trialkylamines, alkylformates, trialkylamines, or a combination thereof. Aliphatic hydrocarbon solvents can be pentane, hexane, heptane, octane, cyclohexane, and the like or a combination thereof. Aromatic hydrocarbon solvents can be benzene, toluene, and the like or a combination thereof. Alcohols or alkylalcohols include, for example, methanol, ethanol, propanol, butanol, pentanol, hexanol, heptanol, octanol, decanol, amylalcohol, or a combination thereof. Alkylethers include methyl, ethyl, propyl, butyl, and the like, e.g., diethylether, diisopropylether or a combination thereof. Sulfoxides include dimethyl sulfoxide (DMSO), decylmethyl sulfoxide, tetradecylmethyl sulfoxide, and the like or a combination thereof. The term “alkylketone” refers to a ketone substituted with an alkyl group, e.g., acetone, ethylmethylketone, and the like or a combination thereof. The term “alkylacetate” refers to an acetate substituted with an alkyl group, e.g., ethylacetate, propylacetate (n-propylacetate, iso-propylacetate), butylacetate (n-butylacetate, iso-butylacetate, sec-butylacetate, tert-butylacetate), amylacetate (n-pentylacetate, tert-pentylacetate, neo-pentylacetate, iso-pentylacetate, sec-pentylacetate, 3-pentylacetate, sec-iso-pentylacetate, active-pentylacetate), 2-ethylhexylacetate, and the like or a combination thereof. The term “alkylformate” refers to a formate substituted with an alkyl group, e.g., methylformate, ethylformate, propylformate, butylformate, and the like or a combination thereof. The term “trialkylamine” refers to an amino group substituted with three alkyl groups, e.g., triethylamine.

As used herein, an “amino acid” or “residue” refers to any naturally or non-naturally occurring amino acid, any amino acid derivative or any amino acid mimic known in the art. Included are the L- as well as the D-forms of the respective amino acids, although the L-forms are usually preferred. In some embodiments, the term relates to any one of the 20 naturally occurring amino acids: glycine (Gly), alanine (Ala), valine (Val), leucine (Leu), isoleucine (Ile), proline (Pro), cysteine (Cys), methionine (Met), serine (Ser), threonine (Thr), glutamine (Gin), asparagine (Asn), glutamic acid (Glu), aspartic acid (Asp), lysine (Lys), histidine (His), arginine (Arg), phenylalanine (Phe), tryptophan (Trp), and tyrosine (Tyr) in their L-form. In certain embodiments, the amino acid side-chain may be a side-chain of Gly, Ala, Val, Leu, Ile, Met, Cys, Ser, Thr, Trp, Phe, Lys, Arg, His, Tyr, Asn, Gln, Asp, Glu, or Pro.

As used herein, except where the context requires otherwise, the term “comprise” and variations of the term, such as “comprising”, “comprises” and “comprised”, are not intended to exclude further additives, components, integers or steps. The terms “including” and “comprising” may be used interchangeably. As used herein, the phrases “selected from the group consisting of”, “chosen from”, and the like, include mixtures of the specified materials. Where a numerical limit or range is stated herein, the endpoints are included. Also, all values and subranges within a numerical limit or range are specifically included as if explicitly written herein. References to an element in the singular is not intended to mean “one and only one” unless specifically stated, but rather “one or more”. Unless specifically stated otherwise, terms such as “some” refer to one or more, and singular terms such as “a”, “an” and “the” refer to one or more.

The term “oligopeptide” is used to refer to a peptide with fewer members of amino acids as opposed to a polypeptide or protein. Oligopeptides described herein, are typically comprised of about two to about forty amino acid residues. Oligopeptides include dipeptides (two amino acids), tripeptides (three amino acids), tetrapeptides (four amino acids), pentapeptides (five amino acids), hexapeptides (six amino acids), heptapeptides (seven amino acids), octapeptides (eight amino acids), nonapeptides (nine amino acids), decapeptides (ten amino acids), undecapeptides (eleven amino acids), dodecapeptides (twelve amino acids), icosapeptides (twenty amino acids), tricontapeptides (thirty amino acids), tetracontapeptides (forty amino acids), etc. Oligopeptides may also be classified according to molecular structure: aeruginosins, cyanopeptolins, microcystins, microviridins, microginins, anabaenopeptins and cyclamides, etc. Homo-oligopeptides are oligopeptides comprising the same amino acid. In preferred embodiments, homo-oligopeptides comprise 10 amino acid poly-valine, poly-alanine, and poly-glycine hexamers.

The meaning of the term “peptides” are defined as small proteins of two or more amino acids linked by the carboxyl group of one to the amino group of another. Accordingly, at its basic level, peptide synthesis of whatever type comprises the repeated steps of adding amino acid or peptide molecules to one another or to an existing peptide chain. The term “peptide” generally has from about 2 to about 100 amino acids, whereas a polypeptide or protein has about 100 or more amino acids, up to a full length sequence which may be translated from a gene. Additionally, as used herein, a peptide can be a subsequence or a portion of a polypeptide or protein. In certain embodiments, the peptide consists of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acid residues. In preferred embodiments, the peptide is from about 30 to about 100 amino acids in length. In some embodiments, the peptide is from about 40 to about 100 amino acids in length.

As used herein, the term “pharmaceutically acceptable” refers to compositions that are physiologically tolerable and do not typically produce an allergic or similar untoward reaction when administered to a subject, preferably a human subject. Preferably, as used herein, the term “pharmaceutically acceptable” means approved by a regulatory agency of a federal or state government or listed in the U.S. Pharmacopeia or other generally recognized pharmacopeia for use in animals, and more particularly in humans.

As used herein, the term “prodrug” is intended to encompass therapeutic biologics which, under physiologic conditions, are converted into the therapeutically active biologics of the present disclosure. A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present disclosure. In certain embodiments, some or all of the molecules in a composition represented above can be replaced with the corresponding suitable prodrug, e.g., wherein a hydroxyl in the parent molecule is presented as an ester or a carbonate or carboxylic acid present in the parent therapeutic biologic is presented as an ester.

The meaning of the term “protein” is defined as a linear polymer built from about 20 different amino acids. The type and the sequence of amino acids in a protein are specified by the DNA that produces them. In certain embodiments, the sequences can be natural and unnatural. The sequence of amino acids determines the overall structure and function of a protein. In some embodiments, proteins can contain 50 or more residues. In preferred embodiments, proteins can contain greater than about 101 residues in length. A protein's net charge can be determined by two factors: 1) the total count of acidic amino acids vs. basic amino acids; and 2) the specific solvent pH surroundings, which expose positive or negative residues. As used herein, “net positively or net negatively charged proteins” are proteins that, under non-denaturing pH surroundings, have a net positive or net negative electric charge. In general, those skilled in the art will recognize that all proteins may be considered “net negatively charged proteins”, regardless of their amino acid composition, depending on their pH and/or solvent surroundings. For example, different solvents can expose negative or positive side chains depending on the solvent pH. Proteins or peptides are preferably selected from any type of enzyme or antibodies or fragments thereof showing substantially the same activity as the corresponding enzyme or antibody. Proteins or peptides may serve as a structural material (e.g. keratin), as enzymes, as hormones, as transporters (e.g. hemoglobin), as antibodies, or as regulators of gene expression. Proteins or peptides are required for the structure, function, and regulation of cells, tissues, and organs.

The term “substantially” as used herein, refers to a majority of, or mostly, as in at least about 50%, 60%, 70%, 80%, 90%, 95%, 96%, 97%, 98%, 99%, 99.5%, 99.9%, 99.99%, or at least about 99.999% or more.

It is understood that the specific order or hierarchy of steps in the methods or processes disclosed is an illustration of exemplary approaches. Based upon design preferences, it is understood that the specific order or hierarchy of steps in the methods or processes may be rearranged. Some of the steps may be performed simultaneously. The accompanying methods claims present elements of the various steps in a sample order, and are not meant to be limited to a specific hierarchy or order presented. A phrase such as “embodiment” does not imply that such embodiment applies to all configurations of the subject technology. A disclosure relating to an embodiment may apply to all embodiments, or one or more embodiments. A phrase such as an embodiment may refer to one or more embodiments and vice-versa.

### Particles

Unless otherwise defined, all terms of art, notations and other scientific terminology used herein, are intended to have the meanings commonly understood by those of skill in the art to which this disclosure pertains. In some cases, terms with commonly understood meanings are defined herein, for clarity and/or for ready reference, and the inclusion of such definitions herein, should not necessarily be construed to represent a substantial difference over what is generally understood in the art. The techniques and procedures described or referenced herein, are generally well understood and commonly employed using conventional methodology by those skilled in the art. As appropriate, procedures involving the use of commercially available kits and reagents are generally carried out in accordance with manufacturer defined protocols and/or parameters unless otherwise noted. As used herein, the phrase “and/or” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The terms “particle” or “particles” or “microparticle” or “microparticles” are used herein, interchangeably in the broadest sense, refers to a discrete body or bodies. The particles described herein, are circular, spheroidal and of controlled dispersity with a characteristic size from sub-micrometers to tens of micrometers, in contrast to, e.g., a porous monolithic “cake”, which is typically produced during conventional lyophilization. This morphology allows for a flowable powder (as described by low Hausner ratios) without post-processing. In some embodiments, the term “particle” refers to a quantity of a therapeutic biologic or therapeutic biologics which is either in a state of matter that is substantially solid as compared to a liquid droplet or in a gel form. The term “proto-particle” refers to a stage of particle formation in which one or more of the components comprising the particle are in an at least a partial state of desiccation. The total liquid content of the proto-particle is less than that of the droplet and greater than that of the formed particle. Similarly, the average concentration of the solutes is higher than that of the drop but typically less than that of the formed particle. The term “encapsulant” refers to a substance that can be dried or gelled around a particle core to form a shell.

As disclosed herein, a therapeutic biologic, also known as a biologic medical product, or biopharmaceutical, is any pharmaceutical drug product manufactured in, extracted from, or semisynthesized from biological sources. Therapeutic biologics can include a wide range of products such as vaccines, blood and blood components, allergenics, somatic cells, gene therapy, tissues, and recombinant therapeutic proteins. In some embodiments, the biologics can be composed of sugars, proteins, or nucleic acids or complex combinations of these substances, or may be living entities such as cells and tissues. Biologics can be isolated from a variety of natural sources, e.g., a human, animal, or microorganism, and may be produced by biotechnology methods or other technologies. Gene-based and cellular biologics, for example, are often used to treat a variety of medical conditions for which no other treatments are available. In preferred embodiments of the disclosure, the therapeutic biologic is an antibody, bovine serum albumin (BSA), or human serum albumin (HSA).

The terms “antibody” and “immunoglobulin” are used interchangeably in the broadest sense and include monoclonal antibodies, polyclonal antibodies, multivalent antibodies, and multispecific antibodies, regardless of how they are produced (i.e., using immunization, recombinant, synthetic methodologies). Antibodies can be gamma globulin proteins that are found in blood, or other bodily fluids of vertebrates that function in the immune system to bind antigen, hence identifying and/or neutralizing foreign objects. Antibodies can be assigned to different classes or isotypes. There are five classes of immunoglobulins: IgA, IgD, IgE, IgG, and IgM, having heavy chains designated alpha, delta, epsilon, gamma, and mu, respectively. The gamma class is further divided into subclasses based on the differences in sequences and function, e.g., humans express the following subclasses: IgG1, IgG2, IgG3, IgG4, IgA1, and IgA2. In certain embodiments of the disclosure, the IgG antibody is an IgG1 antibody. In preferred embodiments of the disclosure, the IgG1 antibody is a monoclonal IgG1 antibody. The L chain from any vertebrate species can be assigned to one of two clearly distinct types, e.g., kappa and lambda, based on the amino acid sequences of their constant domains.

The recognized immunoglobulin genes include the kappa, lambda, alpha, gamma, delta, epsilon and mu constant region genes, as well as the myriad immunoglobulin variable region genes. In some embodiments, light chains are classified as either kappa or lambda. In other embodiments, heavy chains are classified as gamma, mu, alpha, delta, or epsilon, which in turn define the immunoglobulin classes, IgG, IgM, IgA, IgD and IgE, respectively. In preferred embodiments of the disclosure, the antibody is an IgG antibody.

An exemplary antibody (immunoglobulin) structural unit comprises a tetramer. Each tetramer is composed of two identical pairs of polypeptide chains, each pair having one “light” (about sss25 kD) and one “heavy” chain (about 50-70 kD). The N-terminus of each chain defines a variable region of about 100 to 110 or more amino acids primarily responsible for antigen recognition. The terms “variable light” chain, domain, region and component are used interchangeably, are abbreviated by “VL” or “V_{L}” and refer to the light chain of an antibody or antibody fragment. Similarly, terms “variable heavy” chain, domain, region and component are used interchangeably, are abbreviated by “VH” or “V_{H}” and refer to the heavy chain of an antibody or antibody fragment. Antibodies are generally a heterotetrameric glycoprotein composed of two identical light (L) chains and two identical heavy (H) chains. Each L chain is linked to a H chain by one covalent disulfide bond. The two H chains are linked to each other by one or more disulfide bonds depending on the H chain isotype. Each H and L chain also has regularly spaced intra-chain disulfide bridges. H and L chains define specific Ig domains. In particular, each H chain has at the N-terminus, a variable domain (VH) followed by three constant domains (CH) for each of the alpha and gamma chains and four CH domains for p and c isotypes. Each L chain has at the N-terminus, a variable domain (VL) followed by a constant domain (CL) at its other end. The VL is aligned with the VH and the CL is aligned with the first constant domain of the heavy chain (CHL). The constant domain includes the Fc portion which comprises the carboxy-terminal portions of both H chains held together by disulfides. The effector functions of antibodies such as ADCC are determined by sequences in the Fc region, which is also the part recognized by Fc receptors (FcR) found on certain types of cells.

As disclosed herein, the pairing of a VH and VL together form a “variable region” or “variable domain” including the amino-terminal domains of the heavy or light chain of the antibody. The variable domain of the heavy chain may be referred to as “VH”. The variable domain of the light chain may be referred to as “VL”. The V domain contains an “antigen binding site” which affects antigen binding and defines specificity of a particular antibody for its particular antigen. V regions span about 110 amino acid residues and consist of relatively invariant stretches called framework regions (FRs) (generally about 4) of 15-30 amino acids separated by shorter regions of extreme variability called “hypervariable regions” (generally about 3) that are each generally 9-12 amino acids long. The FRs largely adopt a p-sheet configuration and the hypervariable regions form loops connecting, and in some cases forming part of, the p-sheet structure. In certain embodiments, the “hypervariable region” refers to the regions of an antibody variable domain which are hypervariable in sequence and/or form structurally defined loops. Generally, antibodies comprise six hypervariable regions; three in the VH (H1, H2, H3), and three in the VL (L1, L2, L3). “Framework” or “FR” residues are those variable domain residues other than the hypervariable region residues defined herein.

The terms “full length antibody”, “intact antibody” and “whole antibody” are used herein, interchangeably, to refer to an antibody in its substantially intact form, not as antibody fragments as defined above. The terms particularly refer to an antibody with heavy chains that contain the Fc region. A full length antibody can be a native sequence antibody or an antibody variant. In certain embodiments, an “intact” or “whole” antibody is one which comprises an antigen-binding site as well as a CL and at least heavy chain constant domains, CH1, CH2 and CH3. The constant domains may be native sequence constant domains (e.g. human native sequence constant domains) or amino acid sequence variants thereof.

As disclosed herein, “whole antibody fragments including a variable domain” include Fab, Fab′, F(ab′)₂, and Fv fragments; diabodies; linear antibodies, single-chain antibody molecules; and multispecific antibodies formed from antibody fragments. The “Fab fragment” consists of an entire L chain along with the variable region domain of the H chain (VH), and the first constant domain of one heavy chain (CHI). Each Fab fragment is monovalent with respect to antigen binding, i.e., it has a single antigen-binding site. A “Fab′ fragment” differs from Fab fragments by having additional few residues at the carboxy terminus of the CHI domain including one or more cysteines from the antibody hinge region. Fab′-SH is the designation herein for Fab′ in which the cysteine residue(s) of the constant domains bear a free thiol group. A “F(ab′)₂ fragment” roughly corresponds to two disulfide linked Fab fragments having divalent antigen-binding activity and is still capable of cross-linking antigen. An “Fv” is the minimum antibody fragment which contains a complete antigen-recognition and binding site. This fragment consists of a dimer of one heavy and one light chain variable region domain in tight, non-covalent association. In a single-chain Fv (scFv) species, one heavy and one light chain variable domain can be covalently linked by a flexible peptide linker such that the light and heavy chains can associate in a “dimeric” structure analogous to that in a two-chain Fv species. From the folding of these two domains emanate six hypervariable loops (3 loops each from the H and L chain) that contribute the amino acid residues for antigen binding and confer antigen binding specificity to the antibody. “Single-chain Fv” also abbreviated as “sFv” or “scFv” are antibody fragments that comprise the VH and VL antibody domains connected to form a single polypeptide chain. In preferred embodiments, the scFv polypeptide further comprises a polypeptide linker between the VH and VL domains which enables the scFv to form the desired structure for antigen binding. In some embodiments, a “single variable domain” is half of an Fv (comprising only three CDRs specific for an antigen) that has the ability to recognize and bind antigen, although at a lower affinity than the entire binding site.

In some embodiments, “diabodies” refer to antibody fragments with two antigen-binding sites, which fragments comprise a heavy-chain variable domain (VH) connected to a light-chain variable domain (VL) in the same polypeptide chain (VH-VL). The small antibody fragments are prepared by constructing sFv fragments with short linkers (about 5-10 residues) between the VH and VL domains such that inter-chain but not intra-chain pairing of the V domains is achieved, resulting in a bivalent fragment, i.e., fragment having two antigen-binding sites. In other embodiments, diabodies may be bivalent or bispecific. In certain embodiments, bispecific diabodies are heterodimers of two “crossover” sFv fragments in which the VH and VL domains of the two antibodies are present on different polypeptide chains. Triabodies and tetrabodies are also generally known in the art.

“Antigen binding fragments” of antibodies as described herein, comprise only a portion of an intact antibody, generally including an antigen binding site of the intact antibody and thus retaining the ability to bind antigen. Exemplary examples of antibody fragments encompassed by the present definition include but are not limited to: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment which consists of a VH domain; (vii) isolated CDR regions; (viii) F(ab′)₂ fragments, a bivalent fragment including two Fab′ fragments linked by a disulfide bridge at the hinge region; (ix) single chain antibody molecules (e.g. single chain Fv; scFv); (x) “diabodies” with two antigen binding sites, comprising a heavy chain variable domain (VH) connected to a light chain variable domain (VL) in the same polypeptide chain; (xi) “linear antibodies” comprising a pair of tandem Fd, segments (VH-CH1-VH-CH1) which, together with complementary light chain polypeptides, form a pair of antigen binding regions. In some embodiments, an “antigen binding site” generally refers to a molecule that includes at least the hypervariable and framework regions that are required for imparting antigen binding function to a V domain. An antigen binding site may be in the form of an antibody or an antibody fragment, (such as a dAb, Fab, Fd, Fv, F(ab′)₂ or scFv) in a method described herein. In some embodiments, an antigen-binding fragment competes with intact antibody, e.g., with the intact antibody from which the fragment was derived, for antigen binding.

The term “fragment” refers to a part or portion of an antibody or antibody chain comprising fewer amino acid residues than an intact or complete antibody or antibody chain. As used herein, the term “fragment” of an antibody molecule includes Fc fragments and antigen-binding fragments of antibodies, for example, an antibody light chain variable domain (VL), an antibody heavy chain variable domain (VH), a single chain antibody (scFv), a F(ab′)2 fragment, a Fab fragment, an Fd fragment, an Fv fragment, a single domain antibody fragment (DAb), a one-armed (monovalent) antibody, or any antigen-binding molecule formed by combination, assembly or conjugation of such antigen binding fragments.

In some embodiments, the term “single-chain Fv” or “scFv” or “single chain” antibody can refer to antibody fragments comprising the VH and VL domains of antibody, wherein these domains are present in a single polypeptide chain. Generally, the Fv polypeptide further comprises a polypeptide linker between the VH and VL domains which enables the sFv to form the desired structure for antigen binding. For a review of sFv, see Pluckthun, THE PHARMACOLOGY OF MONOCLONAL ANTIBODIES, vol. 113, Rosenburg and Moore eds. Springer-Verlag, New York, pp. 269-315 (1994).

As used herein, the term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies (mAbs) are highly specific, being directed against a single antigenic site or determinant on the antigen. In addition to their specificity, the monoclonal antibodies are advantageous in that they may be synthesized uncontaminated by other antibodies. Monoclonal antibodies may be prepared by the hybridoma methodology. The monoclonal antibodies may also be isolated from phage antibody libraries using molecular engineering techniques. The monoclonal antibodies of the disclosure may be generated by recombinant DNA methods, and are sometimes referred to as “recombinant antibodies” or “recombinant monoclonal antibodies” as described herein. In some embodiments, a monoclonal antibody is a single species of antibody wherein every antibody molecule recognizes the same epitope because all antibody producing cells are derived from a single B-lymphocyte cell line. The methods for generating monoclonal antibodies (mAbs) generally begin along the same lines as those for preparing polyclonal antibodies. In other embodiments, rodents such as mice and rats are used in generating monoclonal antibodies. In certain embodiments, rabbit, sheep, or frog cells are used in generating monoclonal antibodies. The use of rats is well known and may provide certain advantages. Mice (e.g., BALB/c mice) are routinely used and generally give a high percentage of stable fusions. In still other embodiments of the disclosure, the antibody is a monoclonal antibody. In preferred embodiments of the disclosure, the IgG antibody is monoclonal.

In other embodiments, recombinant antibody fragments may be isolated from phage antibody libraries using techniques well known in the art. See, for example, Clackson et al., 1991, Nature 352: 624-628; Marks et al., 1991, J. Mol. Biol. 222: 581-597. Recombinant antibody fragments may be derived from large phage antibody libraries generated by recombination in bacteria (Sblattero and Bradbury, 2000, Nature Biotechnology 18:75-80; and as described herein). Polynucleotides encoding the VH and VL components of antibody fragments (i.e., scFv) may be used to generate recombinant full length immunoglobulins using methods known in the art (see, for example, Persic et al., 1997, Gene 187: 9-18).

An “isolated antibody” is one that has been identified and separated and/or recovered from a component of its pre-existing environment. Contaminant components are materials that would interfere with therapeutic uses for the antibody, and may include enzymes, hormones, and other proteinaceous or nonproteinaceous solutes.

As used herein, a “human antibody” refers to an antibody that possesses an amino acid sequence that corresponds to that of an antibody produced by a human. Human antibodies can be produced using various techniques known in the art, including phage-display libraries. Human antibodies can be prepared by administering the antigen to a transgenic animal that has been modified to produce such antibodies in response to antigenic challenge, but whose endogenous loci has been disabled. “Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin.

An “affinity matured” antibody is one with one or more alterations in one or more hypervariable region thereof that result in an improvement in the affinity of the antibody for antigen, compared to a parent antibody that does not possess those alterations. In some embodiments, affinity matured antibodies can have micromolar affinities for the target antigen. In other embodiments, affinity matured antibodies can have nanomolar or even picomolar affinities for the target antigen. Affinity matured antibodies are produced by procedures known in the art.

A “blocking” antibody or an “antagonist” antibody is one that inhibits or reduces biological activity of the antigen it binds. In some embodiments, blocking antibodies or antagonist antibodies substantially or completely inhibit the biological activity of the antigen. An “agonist antibody”, as used herein, is an antibody, which mimics at least one of the functional activities of a polypeptide of interest.

“Binding affinity” generally refers to the strength of the sum total of noncovalent interactions between a single binding site of a molecule, e.g., an antibody, and its binding partner, e.g., an antigen. Unless indicated otherwise, as used herein, “binding affinity” refers to intrinsic binding affinity, which reflects a 1:1 interaction between members of a binding pair, e.g., antibody and antigen. The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. “Epitope” generally refers to that part of an antigen that is bound by the antigen binding site of an antibody. In some embodiments, an epitope may be “linear” in the sense that the hypervariable loops of the antibody CDRs that form the antigen binding site bind to a sequence of amino acids as in a primary protein structure. In other embodiments, the epitope is a “conformational epitope”, i.e. one in which the hypervariable loops of the CDRs bind to residues as they are presented in the tertiary or quaternary protein structure.

In some embodiments of the foregoing methods, the therapeutic biologic is an antibody. In other embodiments, the antibody includes but are not limited to 3F8, Abagovomab, Abatacept, Abciximab, Abituzumab, Abrezekimab, Abrilumab, Acritumomab, Actoxumab, Abituzumab, Adalimumab-adbm, Adalimumab-atto, Adalimumab-bwwb, Adecatumumab, Ado-trastuzumab emtansine, Aducanumab, Afasevikumab, Afelimomab, Aflibercept, Afutuzumab, Alacizumab pegol, ALD518, Alefacept, Alemtuzumab, Alirocumab, Altumomab pentetate, Amatuximab, Anatumomab mafenatox, Andecaliximab, Anetumab ravtansine, Anifrolumab, Anrukinzumab, Ansuvimab, Apolizumab, Aprutumab ixadotin, Arcitumomab, Ascrinvacumab, Aselizumab, Atezolizumab, Atidortoxumab, Atinumab, Atlizumab, Atoltivimab, Atorolimumab, Avelumab, Azintuxizumab vedotin, Bapineuzumab, Basiliximab, Bavituximab, BCD-100, Bectumomab, Begelomab, Belantamab mafodotin, Belatacept, Belimumab, Bemarituzumab, Benralizumab, Bermekimab, Bersanlimab, Bertilimumab, Besilesomab, Bevacizumab, Bevacizumab-awwb, Bezlotoxumab, Biciromab, Bimagrumab, Bimekizumab, Birtamimab, Bivatuzumab mertansine, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Camidanlumab tesirine, Camrelizumab, Canakinumab, Cantuzumab mertansine, Cantuzumab ravtansine, Caplacizumab, Capromab pendetide, Carlumab, Carotuximab, Catumaxomab, cBR96-doxorubicin immunoconjugate, Cedelizumab, Cemiplimab, Cergutuzumab amunaleukin, Cergutuzumab amunaleukin, Certolizumab pegol, Cetrelimab, Cetuximab, Cibisatamab, Cirmtuzumab, Citatuzumab bogatox, Cixutumumab, Clazakizumab, Clenoliximab, Clivatuzumab tetraxetan, Codrituzumab, Cofetuzumab pelidotin, Coltuximab ravtansine, Conatumumab, Concizumab, Cosfroviximab, Crenezumab, CR6261, Crizanlizumab, Crotedumab, Cusatuzumab, Dacetuzumab, Daclizumab, Dalotuzumab, Dapirolizumab pegol, Daratumumab, Dectrekumab, Demcizumab, Denileukin diftitox, Denintuzumab mafodotin, Denosumab, Depatuxizumab mafodotin, Derlotuximab biotin, Detumomab, Dezamizumab, Dinutuximab, Diridavumab, Domagrozumab, Dorlimomab aritox, Dostarlimab, Drozitumab, DS-8201, Duligotumab, Dupilumab, Durvalumab, Dusigitumab, Duvortuxizumab, Ecromeximab, Eculizumab, Edobacomab, Edrecolomab, Efalizumab, Efungumab, Eldelumab, Elezanumab, Elgemtumab, Elotuzumab, Elsilimomab, Emactuzumab, Emibetuzumab, Emicizumab, Enapotamab vedotin, Enavatuzumab, Enfortumab vedotin, Enlimomab pegol, Enoblituzumab, Enokizumab, Enoticumab, Ensituximab, Epitumomab cituxetan, Epoetin-alfa, Epoetin-alfa-epbx, Epratuzumab, Eptinezumab, Erenumab, Erlizumab, Ertumaxomab, Etanercept, Etanercept-szzs, Etaracizumab, Etigilimab, Etrolizumab, Evinacumab, Evolocumab, Exbivirumab, Factor VIII Fc fusion protein, Factor IX Fc fusion protein, Fanolesomab, Faralimomab, Faricimab, Farletuzumab, Fasinumab, Felvizumab, Fezakinumab, Fibatuzumab, Ficlatuzumab, Figitumumab, Filgrastim, Filgrastim-sndz, Firivumab, Flanvotumab, Fletikumab, Flotetuzumab, Fontolizumab, Foralumab, Foravirumab, Fremanezumab, Fresolimumab, Frovocimab, Frunevetmab, Fulranumab, Futuximab, Galcanezumab, Galiximab, Ganitumab, Gantenerumab, Gatipotuzumab, Gavilimomab, Gedivumab, Gemtuzumab ozogamicin, Gevokizumab, Gilvetmab, Gimsilumab, Girentuximab, Glembatumumab vedotin, Golimumab, Gomiliximab, Gosuranemab, Guselkumab, Ibalizumab, IBI308, Ibritumomab tiuxetan, Icrucumab, Idarucizumab, Ifabotuzumab, Igovomab, Iladatuzumab vedotin, IMAB362, Imalumab, Imaprelimab, Imciromab, Imgatuzumab, Inclacumab, Indatuximab ravtansine, Indusatumab vedotin, Inebilizumab, Infliximab, Infliximab-abda, Infliximab-dyyb, Infliximab-qbtx, Intetumumab, Inolimomab, Inotuzumab ozogamicin, Ipilimumab, Iomab-B, Iratumumab, Isatuximab, Iscalimab, Istiratumab, Itolizumab, Ixekizumab, Keliximab, Labetuzumab, Lacnotuzumab, Ladiratuzumab vedotin, Lambrolizumab, Lampalizumab, Lanadelumab, Landogrozumab, Laprituximab emtansine, Larcaviximab, Lebrikizumab, Lemalesomab, Lendalizumab, Lenvervimab, Lenzilumab, Lerdelimumab, Leronlimab, Lesofavumab, Letolizumab, Lexatumumab, Libivirumab, Lifastuzumab vedotin, Ligelizumab, Loncastuximab tesirine, Losatuxizumab vedotin, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Lupartumab amadotin, Lutikizumab, Maftivimab, Mapatumumab, Margetuximab, Marstacimab, Maslimomab, Mavrilimumab, Matuzumab, Mepolizumab, Metelimumab, Milatuzumab, Minretumomab, Mirikizumab, Mirvetuximab soravtansine, Mitumomab, Modotuximab, Mogamulizumab, Monalizumab, Morolimumab, Mosunetuzumab, Motavizumab, Moxetumomab pasudotox, Muromonab-CD3, Nacolomab tafenatox, Namilumab, Naptumomab estafenatox, Naratuximab emtansine, Narnatumab, Natalizumab, Navicixizumab, Navivumab, Naxitamab, Nebacumab, Necitumumab, Nemolizumab, NEOD001, Nerelimomab, Nesvacumab, Netakimab, Nimotuzumab, Nirsevimab, Nivolumab, Nofetumomab merpentan, Obiltoxaximab, Obinutuzumab, Ocaratuzumab, Ocrelizumab, Odesivimab, Odesivimab-ebgn, Odulimomab, Ofatumumab, Olaratumab, Oleclumab, Olendalizumab, Olokizumab, Omalizumab, Omburtamab, OMS721, Onartuzumab, Ontuxizumab, Onvatilimab, Opicinumab, Oportuzumab monatox, Oregovomab, Orticumab, Otelixizumab, Otilimab, Otlertuzumab, Oxelumab, Ozanezumab, Ozoralizumab, Pagibaximab, Palivizumab, Pamrevlumab, Panitumumab, Pankomab, Panobacumab, Parsatuzumab, Pascolizumab, Pasotuxizumab, Pateclizumab, Patritumab, PDR001, Pegfilgrastim-jmdb, Pembrolizumab, Pemtumomab, Perakizumab, Pertuzumab, Pexelizumab, Pidilizumab, Pinatuzumab vedotin, Pintumomab, Placulumab, Plozalizumab, Pogalizumab, Polatuzumab vedotin, Ponezumab, Porgaviximab, Prasinezumab, Prezalizumab, Priliximab, Pritoxaximab, Pritumumab, PRO 140, Quilizumab, Racotumomab, Radretumab, Rafivirumab, Ralpancizumab, Ramucirumab, Ranevetmab, Ranibizumab, Raxibacumab, Ravagalimab, Ravulizumab, Refanezumab, Regavirumab, Relatlimab, Remtolumab, Reslizumab, Rilonacept, Rilotumumab, Rinucumab, Risankizumab, Rituximab, Rituximab-abbs, Rituximab-arrx, Rituximab-pvvr, Rivabazumab pegol, Rivabazumab pegol, Robatumumab, Rmab, Roledumab, Romilkimab, Romiplostim, Romosozumab, Rontalizumab, Rosmantuzumab, Rovalpituzumab tesirine, Rovalpituzumab tesirine, Rovelizumab, Rozanolixizumab, Ruplizumab, Sacituzumab govitecan, Samalizumab, Samrotamab vedotin, Sapelizumab, Sarilumab, Satralizumab (SA237), Satumomab pendetide, Secukinumab, Selicrelumab, Seribantumab, Setoxaximab, Setrusumab, Sevirumab, Sibrotuzumab, SGN-CD19A, SGN-CD33A, SHP647, Sifalimumab, Siltuximab, Simtuzumab, Siplizumab, Sirtratumab vedotin, Sirukumab, Sofituzumab vedotin, Solanezumab, Solitomab, Sonepcizumab, Sontuzumab, Spartalizumab, Stamulumab, Sulesomab, Suptavumab, Sutimlimab, Suvizumab, Suvratoxumab, Tabalumab, Tacatuzumab tetraxetan, Tadocizumab, Tafasitamab, Talacotuzumab, Talizumab, Tamtuvetmab, Tanezumab, Taplitumomab paptox, Tarextumab, Tavolimab, Tefibazumab, Telimomab aritox, Telisotuzumab vedotin, Tenatumomab, Teneliximab, Teplizumab, Tepoditamab, Teprotumumab, Tesidolumab, Tetulomab, Tezepelumab, TGN1412, Tibulizumab, Ticilimumab, Tildrakizumab, Tigatuzumab, Timigutuzumab, Timolumab, Tiragotumab, Tislelizumab, Tisotumab vedotin, TNX-650, Tocilizumab, Tomuzotuximab, Toralizumab, Tosatoxumab, Tositumomab, Tovetumab, Tralokinumab, Trastuzumab, Trastuzumab-anns, Trastuzumab-dkst, Trastuzumab-dttb, Trastuzumab emtansine, Trastuzumab-pkrb, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vadastuximab talirine, Vanalimab, Vandortuzumab vedotin, Vantictumab, Vanucizumab, Vapaliximab, Varisacumab, Varlilumab, Vatelizumab, Vedolizumab, Veltuzumab, Vepalimomab, Vesencumab, Visilizumab, Vobarilizumab, Volociximab, Vonlerolizumab, Vopratelimab, Vorsetuzumab mafodotin, Votumumab, Xentuzumab, XMAB-5574, Zalutumumab, Zanolimumab, Zatuximab, Zenocutuzumab, Ziralimumab, Zolbetuximab (IMAB362, Claudiximab), Ziv-aflibercept, or Zolimomab aritox.

In other embodiments of the foregoing methods, the antibody is monoclonal. In certain embodiments, the monoclonal antibody includes but are not limited to 3F8, 8H9, Abatacept, Abagovomab, Abciximab, Abituzumab, Adalimumab-adbm, Adalimumab-atto, Adalimumab-bwwb, Abrilumab, Actoxumab, Abituzumab, Abrezekimab, Abrilumab, Actoxumab, Adalimumab, Adecatumumab, Ado-trastuzumab emtansine, Aducanumab, Afasevikumab, Afelimomab, Aflibercept, Afutuzumab, Alacizumab pegol, ALD518, Alefacept, Alemtuzumab, Alirocumab, Altumomab pentetate, Amatuximab, Anatumomab mafenatox, Andecaliximab, Anetumab ravtansine, Anifrolumab, Anrukinzumab (IMA-638), Ansuvimab, Apolizumab, Arcitumomab, Ascrinvacumab, Aselizumab, Atezolizumab, Atidortoxumab, Atinumab, Atlizumab (tocilizumab), Atoltivimab, Atorolimumab, Avelumab, Bapineuzumab, Basiliximab, Bevacizumab, Bevacizumab-awwb, BCD-100, Bectumomab, Begelomab, Belatacept, Belimumab, Bemarituzumab, Benralizumab, Bermekimab, Bersanlimab, Bertilimumab, Besilesomab, Bezlotoxumab, Biciromab, Bimagrumab, Bimekizumab, Birtamimab, Bivatuzumab mertansine, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Camrelizumab, Canakinumab, Cantuzumab mertansine, Cantuzumab ravtansine, Caplacizumab, Capromab pendetide, Carlumab, Carotuximab, Catumaxomab, Cedelizumab, Cemiplimab, Certolizumab pegol, Cetrelimab, Cetuximab, Cibisatamab, Cirmtuzumab, Ch.14.18, Citatuzumab bogatox, Cixutumumab, Clazakizumab, Clenoliximab, Clivatuzumab tetraxetan, Codrituzumab, Cofetuzumab pelidotin, Coltuximab ravtansine, Conatumumab, Concizumab, Cosfroviximab, Crenezumab, CR6261, Crizanlizumab, Crotedumab, Cusatuzumab, Dacetuzumab, Daclizumab, Dalotuzumab, Dapirolizumab pegol, Daratumumab, Dectrekumab, Demcizumab, Denileukin diftitox, Denintuzumab mafodotin, Denosumab, Derlotuximab biotin, Detumomab, Dezamizumab, Dinutuximab, Diridavumab, Domagrozumab, Dorlimomab aritox, Dostarlimab, Drozitumab, Duligotumab, Dupilumab, Durvalumab, Dusigitumab, Duvortuxizumab, Ecromeximab, Eculizumab, Edobacomab, Edrecolomab, Efalizumab, Efungumab, Eldelumab, Elezanumab, Elgemtumab, Elotuzumab, Elsilimomab, Emactuzumab, Emibetuzumab, Emicizumab, Enavatuzumab, Enfortumab vedotin, Enlimomab pegol, Enoblituzumab, Enokizumab, Enoticumab, Ensituximab, Epitumomab cituxetan, Epoetin-alfa, Epoetin-alfa-epbx, Epratuzumab, Eptinezumab, Erenumab, Erlizumab, Ertumaxomab, Etanercept, Etanercept-szzs, Etaracizumab, Etigilimab, Etrolizumab, Evinacumab, Evolocumab, Exbivirumab, Factor VIII Fc fusion protein, Factor IX Fc fusion protein, Fanolesomab, Faralimomab, Faricimab, Farletuzumab, Fasinumab, FBTA05, Felvizumab, Fezakinumab, Fibatuzumab, Ficlatuzumab, Figitumumab, Filgrastim, Filgrastim-sndz, Firivumab, Flanvotumab, Fletikumab, Flotetuzumab, Fontolizumab, Foralumab, Foravirumab, Fremanezumab, Fresolimumab, Frovocimab, Frunevetmab, Fulranumab, Futuximab, Galcanezumab, Galiximab, Ganitumab, Gantenerumab, Gatipotuzumab, Gavilimomab, Gedivumab, Gemtuzumab ozogamicin, Gevokizumab, Gilvetmab, Gimsilumab, Girentuximab, Glembatumumab vedotin, Golimumab, Gomiliximab, Gosuranemab, Guselkumab, Ibalizumab, IBI308, Ibritumomab tiuxetan, Icrucumab, Idarucizumab, Ifabotuzumab, Igovomab, IMAB362, Imalumab, Imaprelimab, Imciromab, Imgatuzumab, Inclacumab, Indatuximab ravtansine, Indusatumab vedotin, Inebilizumab, Infliximab, Infliximab-abda, Infliximab-dyyb, Infliximab-qbtx, Intetumumab, Inolimomab, Inotuzumab ozogamicin, Ipilimumab, Iratumumab, Isatuximab, Iscalimab, Istiratumab, Itolizumab, Ixekizumab, Keliximab, Labetuzumab, Lacnotuzumab, Lambrolizumab, Lampalizumab, Lanadelumab, Landogrozumab, Larcaviximab, Lebrikizumab, Lemalesomab, Lendalizumab, Lenvervimab, Lenzilumab, Lerdelimumab, Leronlimab, Lesofavumab, Letolizumab, Lexatumumab, Libivirumab, Lifastuzumab vedotin, Ligelizumab, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Lutikizumab, Maftivimab, Mapatumumab, Margetuximab, Marstacimab, Maslimomab, Mavrilimumab, Matuzumab, Mepolizumab, Metelimumab, Milatuzumab, Minretumomab, Mirikizumab, Mirvetuximab soravtansine, Mitumomab, Modotuximab, Mogamulizumab, Monalizumab, Morolimumab, Mosunetuzumab, Motavizumab, Moxetumomab pasudotox, Muromonab-CD3, Nacolomab tafenatox, Namilumab, Naptumomab estafenatox, Narnatumab, Natalizumab, Navicixizumab, Navivumab, Naxitamab, Nebacumab, Necitumumab, Nemolizumab, NEOD001, Nerelimomab, Nesvacumab, Netakimab, Nimotuzumab, Nirsevimab, Nivolumab, Nofetumomab merpentan, Obiltoxaximab, Obinutuzumab, Ocaratuzumab, Ocrelizumab, Odesivimab, Odesivimab-ebgn, Odulimomab, Ofatumumab, Olaratumab, Oleclumab, Olendalizumab, Olokizumab, Omalizumab, Omburtamab, OMS721, Onartuzumab, Ontuxizumab, Onvatilimab, Opicinumab, Oportuzumab monatox, Oregovomab, Orticumab, Otelixizumab, Otilimab, Otlertuzumab, Oxelumab, Ozanezumab, Pagibaximab, Palivizumab, Pamrevlumab, Panitumumab, Pankomab, Panobacumab, Parsatuzumab, Pascolizumab, Pasotuxizumab, Pateclizumab, Patritumab, PDR001, Pegfilgrastim-jmdb, Pembrolizumab, Pemtumomab, Perakizumab, Pertuzumab, Pexelizumab, Pidilizumab, Pinatuzumab vedotin, Pintumomab, Placulumab, Plozalizumab, Pogalizumab, Polatuzumab vedotin, Ponezumab, Porgaviximab, Prasinezumab, Prezalizumab, Priliximab, Pritoxaximab, Pritumumab, PRO 140, Quilizumab, Tetulomab, Racotumomab, Radretumab, Rafivirumab, Ralpancizumab, Ramucirumab, Ranevetmab, Ranibizumab, Raxibacumab, Ravagalimab, Ravulizumab, Refanezumab, Regavirumab, Relatlimab, Remtolumab, Reslizumab, Rilonacept, Rilotumumab, Rinucumab, Risankizumab-rzaa, Rituximab, Rituximab-abbs, Rituximab-arrx, Rituximab-pvvr, Robatumumab, Rmab, Roledumab, Romilkimab, Romiplostim, Romosozumab, Rontalizumab, Rosmantuzumab, Rovelizumab, Rozanolixizumab, Ruplizumab, Sacituzumab govitecan, Samalizumab, Sarilumab, Satralizumab (SA237), Satumomab pendetide, Secukinumab, Selicrelumab, Seribantumab, Setoxaximab, Setrusumab, Sevirumab, Sibrotuzumab, SGN-CD19A, SGN-CD33A, SHP647, Sifalimumab, Siltuximab, Simtuzumab, Siplizumab, Sirukumab, Sofituzumab vedotin, Solanezumab, Solitomab, Sonepcizumab, Sontuzumab, Spartalizumab, Stamulumab, Sulesomab, Suptavumab, Sutimlimab, Suvizumab, Suvratoxumab, Tabalumab, Tacatuzumab tetraxetan, Tadocizumab, Tafasitamab, Talacotuzumab, Talizumab, Tamtuvetmab, Tanezumab, Taplitumomab paptox, Tarextumab, Tavolimab, Tefibazumab, Telimomab aritox, Tenatumomab, Teneliximab, Teplizumab, Tepoditamab, Teprotumumab, Tesidolumab, Tetulomab (lilotomab), Tezepelumab, TGN1412, Tibulizumab, Ticilimumab (tremelimumab), Tildrakizumab, Tigatuzumab, Timigutuzumab, Timolumab, Tiragotumab, Tislelizumab, TNX-650, Tocilizumab (atlizumab), Tomuzotuximab, Toralizumab, Tosatoxumab, Tositumomab, Tovetumab, Tralokinumab, Trastuzumab, Trastuzumab-anns, Trastuzumab-dkst, Trastuzumab-dttb, Trastuzumab emtansine, Trastuzumab-pkrb, TRBS07, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vanalimab, Vandortuzumab vedotin, Vantictumab, Vanucizumab, Vapaliximab, Varisacumab, Varlilumab, Vatelizumab, Vedolizumab, Veltuzumab, Vepalimomab, Vesencumab, Visilizumab, Vobarilizumab, Volociximab, Vonlerolizumab, Vopratelimab, Vorsetuzumab mafodotin, Votumumab, Xentuzumab, XMAB-5574, Zalutumumab, Zanolimumab, Zatuximab, Zenocutuzumab, Ziralimumab, Zolbetuximab (IMAB362, Claudiximab), Ziv-aflibercept, Zolimomab aritox or the corresponding anti-drug antibody in a sample from a human patient. In preferred embodiments, the monoclonal antibody is Rituximab, Rituximab-abbs, Rituximab-arrx, or Rituximab-pvvr.

In some embodiments, the monoclonal antibody is a biosimilar. In other embodiments, the biosimilar includes but are not limited to Adalimumab-adbm, Adalimumab-atto, Adalimumab-bwwb, Bevacizumab-awwb, Epoetin alfa-epbx, Etanercept-szzs, Infliximab-abda, Infliximab-dyyb, Infliximab-qbtx, Filgrastim-sndz, Odesivimab-ebgn, Pegfilgrastim-jmdb, Pegfilgrastim-bmez, Risankizumab-rzaa, Rituximab-abbs, Rituximab-arrx, Rituximab-pvvr, Trastuzumab-anns, Trastuzumab-dttb, Trastuzumab-pkrb, or Trastuzumab-dkst. In certain embodiments, the active biosimilar substance is Adalimumab, Bevacizumab, Enoxaparin sodium, Epoetin alfa, Epoetin zeta, Etanercept, Filgrastim, Follitropin alfa, Infliximab, Insulin glargine, Insulin lispro, Pegfilgrastim, Risankizumab, Rituximab, Rituximab-abbs, Rituximab-arrx, Rituximab-pvvr, Somatropin, Teriparatide, or Trastuzumab. In some embodiments, the monoclonal antibody is Atoltivimab, Maftivimab, Odesivimab-ebgn, or a combination thereof. In preferred embodiments, the biosimilar is Rituximab, Rituximab-abbs, Rituximab-arrx, or Rituximab-pvvr.

In other embodiments, the targeting moiety is an antibody from an intact polyclonal antibody, an intact monoclonal antibody, an antibody fragment, a single chain Fv (scFv) mutant, a multispecific antibody, a bispecific antibody, a chimeric antibody, a humanized antibody, a human antibody, a fusion protein comprising an antigenic determinant portion of an antibody, or other modified immunoglobulin molecules comprising antigen recognition sites.

In some embodiments, the therapeutic biologic is an immunotherapy. In other embodiments, the immunotherapy is an anti-CD20 antibody. In certain embodiments, the anti-CD20 antibody is rituximab. In certain other embodiments, the therapeutic biologic is an anti-CD20 antibody. As described herein, any antibody capable of binding the CD20 antigen may be used in the methods of the instant disclosure. Antibodies which bind the CD20 antigen include, for example: C2B8 (rituximab; RITUXAN®) (U.S. Pat. No. 5,736,137, expressly incorporated herein by reference); the yttrium-[90]-labeled 2138 murine antibody designated Y2B8 (U.S. Pat. No. 5,736,137, expressly incorporated herein by reference); murine IgG2a 131 optionally labeled with 131 1 to generate the 131 1-B1 antibody (BEXXAR®) (U.S. Pat. No. 5,595,721, expressly incorporated herein by reference); murine monoclonal antibody 1F5 (Press et al. Blood 69(2): 584-591 (1987)); chimeric 2H7 antibody (U.S. Pat. No. 5,677,180 expressly incorporated herein by reference); and monoclonal antibodies L27, G28-2, 93-1 133, B-C1 or NU-B2 available from the International Leukocyte Typing Workshop (Valentine et al., In: Leukocyte TypingIII (McMichael, Ed., p. 440, Oxford University Press (1987)).

In certain embodiments of the disclosure, the anti-CD20 antibody is rituximab. Rituximab is a genetically engineered chimeric murine/human monoclonal antibody. Rituximab is an IgG, kappa immunoglobulin containing murine light and heavy chain variable region sequences and human constant region sequences. Rituximab has a binding affinity for the CD20 antigen of approximately 8.0 nM and is commercially available, e.g., from Genentech (South San Francisco, Calif.).

In some embodiments, the therapeutic biologic is an immunotherapeutic. In other embodiments, the immunotherapeutic is a PD-1 inhibitor such as a PD-1 antibody, a PD-L1 inhibitor such as a PD-L1 antibody, a CTLA-4 inhibitor such as a CTLA-4 antibody, a CSF-1R inhibitor, an IDO inhibitor, an A1 adenosine inhibitor, an A2A adenosine inhibitor, an A2B adenosine inhibitor, an A3A adenosine inhibitor, an arginase inhibitor, or an HDAC inhibitor. In still other embodiments, the immunotherapeutic is a PD-1 inhibitor (e.g., nivolumab, pembrolizumab, pidilizumab, BMS 936559, and MPDL328OA). In some embodiments, the immunotherapy is a PD-L1 inhibitor (e.g., atezolizumab and MEDI4736). In some embodiments, the immunotherapeutic is a CTLA-4 inhibitor (e.g., ipilimumab). In certain other embodiments, the immunotherapeutic is a CSF-1R inhibitor (e.g., pexidartinib and AZD6495). In certain embodiments, the immunotherapeutic is an IDO inhibitor (e.g., norharmane, rosmarinic acid, and alpha-methyl-tryptophan). In some embodiments, the immunotherapeutic is an A1 adenosine inhibitor (e.g., 8-cyclopentyl-1,3-dimethylxanthine, 8-cyclopentyl-1,3-dipropylxanthine, 8-phenyl-1,3-dipropylxanthine, bamifylline, BG-9719, BG-9928, FK-453, FK-838, rolofylline, or N-0861). In other embodiments, the immunotherapeutic is an A2A adenosine inhibitor (e.g., ATL-4444, istradefylline, MSX-3, preladenant, SCH-58261, SCH-412,348, SCH-442,416, ST-1535, VER-6623, VER-6947, VER-7835, viadenant, or ZM-241,385). In still other embodiments, the immunotherapeutic is an A2B adenosine inhibitor (e.g., ATL-801, CVT-6883, MRS-1706, MRS-1754, OSIP-339,391, PSB-603, PSB-0788, or PSB-1115). In certain other embodiments, the immunotherapeutic is an A3A adenosine inhibitor (e.g., KF-26777, MRS-545, MRS-1191, MRS-1220, MRS-1334, MRS-1523, MRS-3777, MRE-3005-F20, MRE-3008-F20, PSB-11, OT-7999, VUF-5574, and SSR161421). In certain embodiments, the immunotherapeutic is an arginase inhibitor (e.g., an arginase antibody, (2s)-(+)-amino-5-iodoacetamidopentanoic acid, NG-hydroxy-L-arginine, (2S)-(+)-amino-6-iodoacetamidohexanoic acid, or (R)-2-amino-6-borono-2-(2-(piperidin-1-yl)ethyl)hexanoic acid. In some embodiments, the immunotherapeutic is an HDAC inhibitor (e.g., valproic acid, SAHA, or romidepsin). In other embodiments, the immunotherapeutic is a toll-like receptor activator. In still other embodiments, the immunotherapy is a RIG-I-like receptor activator. In certain other embodiments, the immunotherapeutic is a stimulator of interferon genes (STING) pathway activator. In certain embodiments, the immunotherapeutic is an Interleukin-1 receptor agonist, e.g., an IL-R1 antagonist. In some embodiments, the immunotherapeutic is a PTEN inhibitor, e.g., a bisperoxovanadium compound. In other embodiments, the immunotherapeutic is a tumor necrosis factor receptor (TNFR), e.g., TNFR-1 or TNFR-2 inhibitor. In certain embodiments, the immunotherapeutic is a Lymphocyte-activation gene 3 (LAG-3) inhibitor, e.g., GSK2831781.

In other embodiments, the therapeutic biologic is ledipasvir/sofosbuvir, insulin glargine, lenalidomide, pneumococcal 13-valent conjugate vaccine, fluticasone/salmeterol, elvitegravir/cobicistat/emtricitabine/tenofovir alafenamide, emtricitabine, rilpivirine and tenofovir alafenamide, emtricitabine/tenofovir alafenamide, grazoprevir/elbasvir, coagulation factor VIIa recombinant, epoetin alfa, Aflibercept or etanercept.

In some embodiments, the therapeutic biologic is Abatacept, AbobotulinumtoxinA, Agalsidase beta, Albiglutide, Aldesleukin, Alglucosidase alfa, Alteplase (cathflo activase), Anakinra, Asfotase alfa, Asparaginase, Asparaginase Erwinia chrysanthemi, Becaplermin, Belatacept, Collagenase, Collagenase clostridium histolyticum, Darbepoetin alfa, Denileukin diftitox, Dornase alfa, Dulaglutide, Ecallantide, Elosulfase alfa, Etanercept-szzs, Filgrastim, Filgrastim-sndz, Galsulfase, Glucarpidase, Idursulfase, IncobotulinumtoxinA, Interferon alfa-2b, Interferon alfa-n3, Interferon beta-1a, Interferon beta-1b, Interferon gamma-1b, Laronidase, Methoxy polyethylene glycol-epoetin beta, Metreleptin, Ocriplasmin, OnabotulinumtoxinA, Oprelvekin, Palifermin, Parathyroid hormone, Pegaspargase, Pegfilgrastim, Peginterferon alfa-2a, Peginterferon alfa-2a co-packaged with ribavirin, Peginterferon alfa-2b, Peginterferon beta-1a, Pegloticase, Rasburicase, Reteplase, Rilonacept, RimabotulinumtoxinB, Romiplostim, Sargramostim, Sebelipase alfa, Tbo-filgrastim, Tenecteplase, or Ziv-aflibercept.

The therapeutic biologic in the particles may have an activity per unit of about 0.5 to about 1.0, about 0.75 to about 1.0 activity per unit, or about 0.9 to about 1.0 activity per unit. Activity is measured relative to the same therapeutic biologic prior to particle formation. In preferred embodiments, the therapeutic biologic has an activity per unit of about 0.5 to about 1.0. The term “activity” refers to the ratio of a functional or structural aspect of an therapeutic biologic, e.g., an antibody, bovine serum albumin (BSA), or human serum albumin (HSA), at two points in time. The denominator of the ratio corresponds to a measure of the functional or structural aspect of the therapeutic biologic in the feed solution, immediately in advance of droplet formation. The numerator of the ratio corresponds to the same measure of a functional or structural aspect of the therapeutic biologic at a later point in time, e.g., immediately after particle formation.

In some embodiments, the particles have less than about 25% internal void spaces, e.g., less than about 24, 20, 15, 10, 9, 8, 7, 6, 5, 4, 3, 2, 1, 0.5, or 0.1% internal void spaces after removing the first liquid, second liquid, and third liquid. In certain embodiments, the particle includes less than about 10% internal void spaces, less than about 5% internal void spaces, less than about 1% internal void spaces, less than about 0.1% internal void spaces, or less than about 0.01% internal void spaces after removing the first liquid, second liquid, and third liquid. In preferred embodiments, the particle is substantially free from any internal void spaces after removing the first liquid, second liquid, and third liquid. Suitable methods for determining internal void space can be accomplished by using Focused Ion Beam Scanning Electron Microscopy (FIB-SEM), which can be used to visualize “accessible” and “inaccessible” void spaces, or gas displacement pycnometry (Micromeritics Instrument Corporation of Norcross, Ga.), which can determine “accessible” voids (void spaces accessible from the surface rather than those resembling a core-shell structure that are “unaccessible form the surface”). Gas pycnometry is a common analytical technique that uses a gas displacement method to measure volume. Inert gases, such as helium or nitrogen, are used as the displacement medium. True volume is total volume minus volume accessible to the gas. Density is calculated by dividing sample weight with true volume. The sample is sealed in the instrument compartment of a known volume, the appropriate inert gas is admitted, and then expanded into another precision internal volume. The pressure before and after expansion is measured and used to compute the sample volume. Dividing this volume into the sample weight gives the gas displacement density. Cross-sections of typical particles of the disclosure indicate an absence of pores (substantially free from any internal void spaces) and low particle porosity as shown by FIB-SEM or by gas pycnometry using helium at temperatures at about 22° C. to provide densities typically averaging about 1.3 g/cm³ with standard deviations at about 0.0005 g/cm³. For example, internal void space can be calculated using the following formula: internal void space=Aᵥ/Aₚ, where Aᵥ is the total area of void spaces and Aₚ is the total area of the particle.

In other embodiments, the particles may exhibit a porosity from about 0 to about 50% after removing the first liquid, second liquid, and third liquid, e.g., from about 0 to about 10%, from about 0 to about 5%, from about 0 to about 1%, from about 0 to about 0.5%, from about 0 to about 0.1%, or from about 0 to about 0.01% after removing the first liquid, second liquid, and third liquid. Exemplary pore size measurements include scanning electron microscopy (SEM), transmission electron microscopy (TEM), and confocal laser scanning microscopy analysis. A gallium focused ion beam (FIB) was used to cut one of the particles in half to reveal a cross-section of the particle interior. The specific surface area of porous micro- and nanospheres may also be investigated by nitrogen adsorption/desorption analysis and a Brunauer-Emmett-Teller adsorption model. In certain embodiments where the pore sizes are sufficiently large, mercury-intrusion porosimetry may be employed.

The particles according to the disclosure are circular. Circularity can serve as an indicator of the shape of the particle. The particles described herein, can have a characteristic circularity, e.g., have a relative shape, that is substantially circular. This characteristic describes and defines the form of a particle on the basis of its circularity. The circularity is 1.0 when the particle has a completely circular structure. Particles as described herein, have a circularity of about 0.8, 0.9, 0.95, 0.96, 0.97, 0.98, or 0.99 after removing the first liquid, second liquid, and third liquid; greater than about 0.80, greater than about 0.90, greater than about 0.95, or greater than about 0.98 after removing the first liquid, second liquid, and third liquid. In some embodiments, the circularity of the particles is greater than about 0.88 after removing the first liquid, second liquid, and third liquid. In other embodiments, the circularity of the particles is greater than about 0.90 after removing the first liquid, second liquid, and third liquid. In certain embodiments, the circularity of the particles is greater than about 0.93 after removing the first liquid, second liquid, and third liquid. In preferred embodiments, the circularity of the particles is greater than about 0.97 after removing the first liquid, second liquid, and third liquid. The diameter and the circularity of the particles can be determined by the image processing of an image observed under an electron microscope or the like or a flow-type particle image analyzer. The circularity can also be determined by subjecting particles to circularity measurement and averaging the resulting values. For example, circularity (circ) can be calculated using the following formula:

circ=4*π*Area/Perimeter². Eq. 1

The term “perimeter”, as used herein, refers to the boundary of a closed plane figure or the sum of all borders of a two-dimensional image. As used herein, the term “area”, refers to the crossectional area of a two-dimensional image of a particle. The circularity of a particle can also be described as the ratio of the smallest diameter of the particle to its largest diameter. For a perfect circle, the ratio is 1. The percentage circularity can be calculated by multiplying the circularity by 100. The circularity can be calculated, for example, by measuring the aspect ratio using any software adapted to deal with images, for example, images obtained by microscopy, in particular, scanning electron microscopy (SEM) or transmission electron microscopy (TEM). In some embodiments, the circularity of the particles is at least about 10% after removing the first liquid, second liquid, and third liquid, e.g., at least about 20, 30, 40, 50, 55, 60, 65, 70, 75, 80, 85, 90, 91, 92, 93, 94, 95, 96, 97, 98, 99, or about 100% after removing the first liquid, second liquid, and third liquid. In other embodiments, the circularity of the particles is at least about 88% after removing the first liquid, second liquid, and third liquid. In certain embodiments, the circularity of the particles is at least about 90% after removing the first liquid, second liquid, and third liquid. In still other embodiments, the circularity of the particles is at least about 93% after removing the first liquid, second liquid, and third liquid. In preferred embodiments, the circularity of the particles is at least about 97% after removing the first liquid, second liquid, and third liquid.

In other embodiments, the circularity of the particles is from about 0.10 to about 1.00 after removing the first liquid, second liquid, and third liquid, e.g., from about 0.20, 0.30, 0.40, 0.50, 0.60, 0.70, 0.75, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, or 0.99 to about 1.00 after removing the first liquid, second liquid, and third liquid. In certain embodiments, the circularity of the particles is from about 0.88 to about 1.00 after removing the first liquid, second liquid, and third liquid. In still other embodiments, the circularity of the particles is from about 0.90 to about 1.00 after removing the first liquid, second liquid, and third liquid. In certain other embodiments, the circularity of the particles is from about 0.93 to about 1.00 after removing the first liquid, second liquid, and third liquid. In preferred embodiments, the circularity of the particles is from about 0.97 to about 1.00 after removing the first liquid, second liquid, and third liquid. In some embodiments, methods of measuring particle circularity include image analysis of scanning electron micrographs of the particles in which the average roundness is calculated on the basis of the cross-sectional shapes of the particles projected onto the plane of the image. Such roundness factors can be extended to identify the corresponding circularity.

In some embodiments of the disclosure, the drying operation may be controlled to provide particles having particular characteristics, such as particles having a substantially smooth surface. “Surface roughness”, as used herein, means a particle having numerous wrinkles or creases, e.g., being ridged or wrinkled. The term “pit”, as used herein, refers to an indentation or crevice in the particle, either an indentation or crevice in the two-dimensional image or an indentation or crevice in an object. The term “spike”, as used herein, refers to a projection pointing outward from the centroid of a particle, a projection pointing outward from the centroid of a two-dimensional image or a sharp projection pointing outward from an object.

In preferred embodiments of the disclosure, the particles as described herein, have a surface morphology that is smooth rather than ridged or wrinkled. The surface roughness of the particles may be decreased by controlling the formulation and/or process to form the particles as described herein. In certain embodiments, the drying conditions can be selected to control the particle morphology in order to enhance the smoothness of the particle's surface. In particular, the drying conditions can be selected to provide particles having a substantially smooth surface. In certain preferred embodiments, the particles have a substantially smooth surface after removing the first liquid, second liquid, and third liquid. A person of ordinary skill in the field of this disclosure can readily assess the surface morphology of the disclosed particles using routine and standard techniques.

In other embodiments, the particle has a diameter of about 0.1 to about 1000 μm after removing the first liquid, second liquid, and third liquid, e.g., about 0.1 to about 900, 800, 700, 600, 500, 400, 300, 200, 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, 1, 0.9, 0.8, 0.7, 0.6, 0.5, 0.4, 0.3, or about 0.2 μm after removing the first liquid, second liquid, and third liquid. In certain embodiments, the particle has a diameter of about 1 to about 100 μm after removing the first liquid, second liquid, and third liquid, e.g., about 2, 3, 4, 5, 6, 7, 8, 9, 10, 11, 12, 13, 14, 15, 16, 17, 18, 19, 20, 25, 30, 35, 40, 45, or 50 to about 100 μm after removing the first liquid, second liquid, and third liquid. In still other embodiments, the particle has a diameter of about 4 to about 100 μm after removing the first liquid, second liquid, and third liquid. In certain other embodiments, the particle has a diameter of about 10 to about 100 μm after removing the first liquid, second liquid, and third liquid. In preferred embodiments, the particle has a diameter of about 20 to about 50 μm after removing the first liquid, second liquid, and third liquid. In certain preferred embodiments, the particle is intentionally controlled in its diameter. In some embodiments, the particles have diameters from about 0.1 to about 1000 μm after removing the first liquid, second liquid, and third liquid, e.g., about 1 to about 400 μm, about 1 to about 200 μm, about 1 to about 100 μm, about 1 to about 50 μm, about 1 to about 25 μm, about 1 to about 10 μm, about 10 to about 100 μm, about 50 to about 100 μm, about 50 to about 75 μm, or about 75 to about 100 μm after removing the first liquid, second liquid, and third liquid. In other embodiments, the particles have diameters from about 1 to about 100 μm after removing the first liquid, second liquid, and third liquid, e.g., from about 4 to about 100 μm, from about 10 to about 100 μm, or from about 20 to about 50 μm after removing the first liquid, second liquid, and third liquid.

In certain embodiments, the particle has a diameter of about 0.1 to about 1000 μm after removing the first liquid, second liquid, and third liquid. In certain other embodiments, the particle has a diameter of about 1 to about 100 μm after removing the first liquid, second liquid, and third liquid. In still other embodiments, the particle has a diameter of about 5 to about 100 μm after removing the first liquid, second liquid, and third liquid. In certain preferred embodiments, the particle has a diameter of about 5 to about 50 μm after removing the first liquid, second liquid, and third liquid. In preferred embodiments, the particle has a diameter of about 5 to about 20 μm after removing the first liquid, second liquid, and third liquid.

In other embodiments, the particles exhibit a skeletal density from about 1.00 to about 6.00 g/cm³ after removing the first liquid, second liquid, and third liquid, e.g., from about 1.00 to about 5.00 g/cm³, from about 1.00 to about 3.00 g/cm³, from about 1.00 to about 2.00 g/cm³, from about 1.00 to about 1.50 g/cm³, from about 1.30 to about 1.50 g/cm³, from about 1.32 to about 1.50 g/cm³, or from about 1.10 to about 1.40 g/cm³ after removing the first liquid, second liquid, and third liquid. In some embodiments, the particles exhibit a skeletal density from about 0.10 to about 5.00 g/cm³ after removing the first liquid, second liquid, and third liquid, e.g., from about 0.10 to about 2.50 g/cm³, from about 0.10 to about 1.40 g/cm³, from about 0.50 to about 1.40 g/cm³, or from about 1.00 to about 1.40 g/cm³ after removing the first liquid, second liquid, and third liquid. In certain embodiments, the particle has a skeletal density of about 1.15 to about 1.60 g/cm³ after removing the first liquid, second liquid, and third liquid. In still other embodiments, the particle has a skeletal density of about 1.25 to about 1.50 g/cm³ after removing the first liquid, second liquid, and third liquid. In preferred embodiments, the particle has a skeletal density of about 1.30 to about 1.40 g/cm³ after removing the first liquid, second liquid, and third liquid. Exemplary methods of skeletal density measurements include gas displacement pycnometry.

In certain embodiments, the particles have a skeletal density of about 1000 mg/mL to about 1500 mg/mL, about 1050 mg/mL to about 1500 mg/mL, about 1100 mg/mL to about 1500 mg/mL, about 1150 mg/mL to about 1500 mg/mL, about 1200 mg/mL to about 1500 mg/mL, about 1250 mg/mL to about 1500 mg/mL, about 1300 mg/mL to about 1500 mg/mL, about 1310 mg/mL to about 1500 mg/mL, about 1320 mg/mL to about 1500 mg/mL, about 1330 mg/mL to about 1500 mg/mL, about 1340 mg/mL to about 1500 mg/mL, about 1350 mg/mL to about 1500 mg/mL, about 1360 mg/mL to about 1500 mg/mL, about 1370 mg/mL to about 1500 mg/mL, about 1380 mg/mL to about 1500 mg/mL, about 1390 mg/mL to about 1500 mg/mL, about 1400 mg/mL to about 1500 mg/mL, about 1410 mg/mL to about 1500 mg/mL, about 1420 mg/mL to about 1500 mg/mL, about 1430 mg/mL to about 1500 mg/mL, about 1440 mg/mL to about 1500 mg/mL, about 1450 mg/mL to about 1500 mg/mL, about 1460 mg/mL to about 1500 mg/mL, about 1470 mg/mL to about 1500 mg/mL, about 1480 mg/mL to about 1500 mg/mL, or about 1490 mg/mL to about 1500 mg/mL after removing the first liquid, second liquid, and third liquid.

In some embodiments, the particles can be characterized by a glass transition temperature of about 0° C. to about 250° C. after removing the first liquid, second liquid, and third liquid, e.g., of about 34° C. to about 200° C., of about 60° C. to about 170° C., of about 90° C. to about 170° C., of about 100 to about 170° C., of about 130 to about 170° C., of about 150 to about 170° C., or of about 160 to about 170° C. after removing the first liquid, second liquid, and third liquid. The term “glass transition” as used herein, refers to a thermodynamic transition of an amorphous material characterized by step changes in specific heat capacity and modulus. At temperatures above the glass transition temperature, molecular mobility is increased as are the rates of physical and chemical changes. Exemplary analytical methods for the determination of the glass transition temperature include differential scanning calorimetry and dynamic mobility analysis. In other embodiments, the particle has a glass transition temperature of about 60 to about 170° C. after removing the first liquid, second liquid, and third liquid. In still other embodiments, the particle has a glass transition temperature of about 90 to about 130° C. after removing the first liquid, second liquid, and third liquid. In certain embodiments, the particle has a glass transition temperature of about 100 to about 130° C. after removing the first liquid, second liquid, and third liquid. In preferred embodiments, the particle has a glass transition temperature of about 120 to about 130° C. after removing the first liquid, second liquid, and third liquid. In certain preferred embodiments, the particle has a glass transition temperature of about 60 to about 120° C. after removing the first liquid, second liquid, and third liquid.

In certain embodiments, the particle has a glass transition temperature that is higher than about 60° C. after removing the first liquid, second liquid, and third liquid. In certain other embodiments, the particle has a glass transition temperature that is higher than about 90° C. after removing the first liquid, second liquid, and third liquid. In still other embodiments, the particle has a glass transition temperature that is higher than about 100° C. after removing the first liquid, second liquid, and third liquid. In certain preferred embodiments, the particle has a glass transition temperature that is higher than about 130° C. after removing the first liquid, second liquid, and third liquid. In preferred embodiments, the particle has a glass transition temperature that is higher than about 170° C. after removing the first liquid, second liquid, and third liquid.

In other embodiments, the particles are heated to about ±30° C., e.g., to about ±20, ±10, ±5, ±1° C., of the glass transition temperature of the particles during drying.

In some embodiments, the particles further comprise a carbohydrate, a pH adjusting agent, a salt, a chelator, a mineral, a polymer, a protein stabilizer, an emulsifier, an antiseptic, an amino acid, an antioxidant, a protein, an organic solvent, a paraben, a bactericide, a fungicide, a vitamin, a preservative, a nutrient media, an oligopeptide, a biologic excipient, a chemical excipient, a surfactant, or a combination thereof.

In other embodiments, the carbohydrate may be from the families of monosaccharides, disaccharides, oligosaccharides, or polysaccharides. In some embodiments, the carbohydrate is dextran, trehalose, sucrose, agarose, mannitol, lactose, sorbitol, maltose, starch, alginates, xanthan, galactomanin, agar, agarose, or a combination thereof. In certain embodiments, the carbohydrate is dextran, trehalose, sucrose, agarose, mannitol, lactose, sorbitol, maltose, or a combination thereof. In preferred embodiments, the carbohydrate is trehalose, cyclodextrins, hydroxypropyl beta-cyclodextrin, sulfobutylether beta-cyclodextrin, or a combination thereof. Cyclodextrins are available in three different forms α, β, and γ based on the number of number of glucose monomers. The number of glucose monomers in α, β, and γcyclodextrin can be 6, 7, or 8, respectively.

In some embodiments, the pH adjusting agent is acetate, citrate, glutamate, glycinate, histidine, lactate, maleate, phosphate, succinate, tartrate, bicarbonate, aluminum hydroxide, phosphoric acid, hydrochloric acid, DL-lactic/glycolic acids, phosphorylethanolamine, tromethamine, imidazole, glyclyglycine, monosodium glutamate, sodium hydroxide, potassium hydroxide, or a combination thereof. In other embodiments, the pH adjusting agent is citrate, histidine, phosphate, succinate, sodium hydroxide, potassium hydroxide, or a combination thereof. In certain embodiments, the pH adjusting agent is hydrochloric acid or citric acid.

In other embodiments, the salt is sodium chloride, calcium chloride, potassium chloride, sodium hydroxide, stannous chloride, magnesium sulfate, sodium glucoheptonate, sodium pertechnetate, guanidine hydrochloride, potassium hydroxide, magnesium chloride, potassium nitrate, or a combination thereof. In preferred embodiments, the salt is sodium chloride.

In some embodiments, the chelator is disodium edetate, ethylenediaminetetraacetic acid, pentetic acid, or a combination thereof. In other embodiments, the mineral is calcium, zinc, titanium dioxide, or a combination thereof. In certain embodiments, the polymer is propyleneglycol, glucose star polymer, silicone polymer, polydimethylsiloxane, polyethylene glycol, carboxymethylcellulose, poly(glycolic acid), poly(lactic-co-glycolic acid), polylactic acid, polycaprolactone (PCL), polyvinylpyrrolidone (PVP), ficoll, dextran, or a combination thereof.

In other embodiments, the protein stabilizer is acetyltryptophanate, caprylate, N-acetyltryptophan, trehalose, polyethylene glycol (PEG), polyoxamers, polyvinylpyrrolidone, polyacrylic acids, poly(vinyl) polymers, polyesters, polyaldehydes, tert-polymers, polyamino acids, hydroxyethylstarch, N-methyl-2-pyrrolidone, sorbitol, sucrose, mannitol, or a combination thereof. In certain embodiments, the protein stabilizer is trehalose, polyethylene glycol (PEG), polyoxamers, polyvinylpyrrolidone, polyacrylic acids, poly(vinyl) polymers, polyesters, polyaldehydes, tert-polymers, polyamino acids, hydroxyethyl starch, N-methyl-2-pyrrolidone, sorbitol, sucrose, mannitol, cyclodextrin, hydroxypropyl beta-cyclodextrin, sulfobutylether beta-cyclodextrin, or a combination thereof. In preferred embodiments, the protein stabilizer is trehalose, cyclodextrin, hydroxypropyl beta-cyclodextrin, sulfobutylether beta-cyclodextrin, or a combination thereof. The stabilizers, used synonymously with the term “stabilizing agent”, as described herein, can be a salt, a carbohydrate, saccharides or amino acids, preferably a carbohydrate or saccharide admitted by the authorities as a suitable additive or excipient in pharmaceutical compositions. In preferred embodiments, the PEG is PEG 200, PEG 300, PEG 3350, PEG 8000, PEG 10000, PEG 20000, or a combination thereof. The term “stabilizer” refers to an excipient or a mixture of excipients which stabilizes the physical and/or chemical properties of a therapeutic biologic, e.g., an antibody. In some embodiments, stabilizers prevent, e.g., degradation of the therapeutic biologic during droplet formation, desiccation, and/or storage of the particulate matter. Exemplary stabilizers include, but are not limited to, sugars, salts, hydrophobic salts, detergents, reducing agents, cyclodextrins, polyols, carboxylic acids, and amino acids. A “stable” formulation as described herein, refers to a formulation in which the therapeutic biologic retains an acceptable portion of its essential physical, chemical, or biological properties over an acceptable period of time. In the case of proteins, e.g., exemplary methods of assessing stability are reviewed in (i) Peptide and Protein Drug Delivery, 247-301, Vincent Lee Ed., Marcel Dekker, Inc., New York, N.Y., 1991, and (ii) Jones, A., Adv. Drug Delivery Rev. 10: 29-90 (1993). In certain embodiments, chemical stability of a protein is assessed by measuring the size distribution of the sample at several stages. These include, e.g., before particle formation (assessment of the feed solution), immediately after particle formation, and again after a period of storage, where storage takes place either within or in the absence of a suspension formulation carrier medium. In certain other embodiments, the size distribution is assessed by size exclusion chromatography (SEC-HPLC). The term “excipient” refers to an additive to a preparation or formulation, which may be useful in achieving a desired modification to the characteristics of the preparation or formulation. Such modifications include, but are not limited to, physical stability, chemical stability, and therapeutic efficacy. Exemplary excipients include, but are not limited to a carbohydrate, a pH adjusting agent, a salt, a chelator, a mineral, a polymer, a surfactant, an amino acid, an oligopeptide, a biologic excipient, a chemical excipient, an antiseptic, an antioxidant, a paraben, a bactericide, a fungicide, a vitamin, a preservative, an analgesic, and/or nutrient media.

Examples of emulsifiers suitable for use in the particles include, but are not limited to, lipophilic agents having an HLB of less than 7, such as mixed fatty acid monoglycerides; mixed fatty acid diglycerides; mixtures of fatty acid mono- and diglycerides; lipophilic polyglycerol esters; glycerol esters including glyceryl monooleate, glyceryl dioleate, glyceryl monostearate, glyceryl distearate, glyceryl monopalmitate, and glyceryl dipalmitate; glyceryl-lacto esters of fatty acids; propylene glycol esters including propylene glycol monopalmitate, propylene glycol monostearate, and propylene glycol monooleate; sorbitan ester including sorbitan monostearate, sorbitan sesquioleate; fatty acids and their soaps including stearic acid, palmitic acid, and oleic acid; and mixtures thereof glyceryl monooleate, glyceryl dioleate, glyceryl monostearate, glyceryl distearate, glyceryl monopalmitate, and glyceryl dipalmitate; glyceryl-lacto esters of fatty acids; propylene glycol esters including propylene glycol monopalmitate, propylene glycol monostearate, and propylene glycol monooleate; sorbitan ester including sorbitan monostearate, sorbitan sesquioleate; fatty acids and their soaps including stearic acid, palmitic acid, and oleic acid; phospholipids; or a combination thereof. In some embodiments, the emulsifier is polysorbate 80, polysorbate 60, polysorbate 20, e.g., Tween 80, Tween 60, Tween 20, sorbitan monooleate, ethanolamine, polyoxyl 35 castor oil, poloxyl 40 hydrogenated castor oil, carbomer 1342, a corn oil-mono-di-triglyceride, a polyoxyethylated oleic glyceride, a poloxamer, or a combination thereof. In preferred embodiments, the fatty acid ester of sorbitol is a sorbitan ester, e.g., span 20, span 40, span 60, or span 80. In certain preferred embodiments, the emulsifier is polysorbate 80, sorbitan monooleate, or a combination thereof.

In other embodiments, the antiseptic is phenol, m-cresol, benzyl alcohol, 2-phenyloxyethanol, chlorobutanol, neomycin, benzethonium chloride, gluteraldehyde, beta-propiolactone, or a combination thereof.

In certain embodiments, the amino acid is alanine, aspartic acid, cysteine, isoleucine, glutamic acid, leucine, methionine, phenylalanine, pyrrolysine, serine, selenocysteine, threonine, tryptophan, tyrosine, valine, asparagine, arginine, histidine, glycine, glutamine, proline, or various salts thereof (arginine hydrochloride, arginine glutamate, etc.) or a combination thereof. In certain other embodiments, the amino acid is alanine, aspartic acid, cysteine, isoleucine, glutamic acid, leucine, methionine, phenylalanine, pyrrolysine, serine, selenocysteine, threonine, tryptophan, tyrosine, valine, asparagine, arginine, histidine, glycine, glutamine, proline, or a combination thereof. In certain preferred embodiments, the amino acid is arginine, histidine, proline, asparagine, or a combination thereof. In preferred embodiments, the amino acid is histidine.

In some embodiments, the antioxidant is glutathione, ascorbic acid, cysteine, N-acety-L-tryptophanate, tocopherol, histidine, methionine, or a combination thereof. In other embodiments, the protein is protamine, protamine sulfate, gelatin, or a combination thereof. In certain embodiments, the organic solvent is dimethyl sulfoxide, N-methyl-2-pyrrolidone, or a combination thereof. The paraben can be a parahydroxybenzoate. In still other embodiments, the bactericide is benzalkonium chloride (cationic surfactants), hypochlorites, peroxides, alcohols, phenolic compounds (e.g. carbolic acid), benzyl benzoate, or a combination thereof. In preferred embodiments, the bactericide is benzyl benzoate.

In other embodiments, the fungicide is acibenzolar, 2-phenylphenol, anilazine, carvone, natamycin, potassium azide, or a combination thereof. In preferred embodiments, the fungicide is benzyl benzoate. In certain embodiments, the vitamin is thiamine, riboflavin, niacin, pantothenic acid, biotin, vitamin B₆, vitamin B₁₂, folate, niacin, ascorbic acid, calciferols, retinols, quinones, or a combination thereof. In still other embodiments, the preservative is sodium nitrate, sulfur dioxide, potassium sorbate, sodium sorbate, sodium benzoate, benzoic acid, methyl hydroxybenzoate, thimerosal, parabens, formaldehyde, castor oil, or a combination thereof. In preferred embodiments, the preservative is methyl hydroxybenzoate, thimerosal, a paraben, formaldehyde, castor oil, or a combination thereof.

A number of nutrient media, preferably serum free, alone or in combination, may be used in the present disclosure, including commercially available media or other media well known in the art. Examples of such media (all without serum or having had the serum removed) include ADC-1, LPM (Bovine Serum Albumin-free), F10 (HAM), F12 (HAM), DCCM1, DCCM2, RPMI 1640, BGJ Medium (Fitton-Jackson Modification), Basal Medium Eagle (BME—with the addition of Earle's salt base), Dulbecco's Modified Eagle Medium (DMEM—without serum), Glasgow Modification Eagle Medium (GMEM), Leibovitz L-15 Medium, McCoy's 5 A Medium, Medium M199 (M199E—with Earle's salt base), Medium M199 (M199H—with Hank's salt base), Minimum Essential Medium Eagle (MEM-E—with Earle's salt base), Minimum Essential Medium Eagle (MEM-H—with Hank's salt base) and Minimum Essential Medium Eagle (MEM-NAA—with non-essential amino acids), among numerous others. In addition, serum-containing nutrient media may also be used in compositions according to the present disclosure, but the use of serum-containing media is less preferred because of the possibility that the serum may be contaminated with microbial agents and because the patient may develop immunological reactions to certain antigenic components contained in the serum.

In some embodiments, the oligopeptide is trileucine. In other embodiments, the biologic excipient are nucleic acids, oligonucleotides, antibodies or fragment thereof, amino acids, polyamino acids, peptides, proteins, cells, bacteria, gene therapeutics, genome engineering therapeutics, epigenome engineering therapeutics, hormones, nucleoproteins, glycoproteins, lipoproteins, exosomes, outer membrane vesicles, vaccines, viruses, bacteriophages, organelles, nutrient media, or a combination thereof. In certain embodiments, the chemical excipient are chemical drugs, contrast agents, dyes, magnetic particles, polymer beads, metal nanoparticles, metal microparticles, quantum dots, antioxidants, antibiotic agents, steroids, analgesics, local anesthetics, anti-inflammatory agents, parabens, anti-microbial agents, chemotherapeutic agents, vitamins, minerals, bactericides, antiseptics, or a combination thereof.

In other embodiments, the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, sodium laureth sulfate, lecithin, or a combination thereof. In some embodiments, the surfactant includes, but is not limited to: (i) cationic surfactants such as; cetyltrimethylammonium chloride, hexadecyltrimethylammonium chloride, benzalkonium chloride, benzethonium chloride, dioctadecyldimethylammonium bromide; (ii) anionic surfactants such as magnesium stearate, sodium dodecyl sulfate, dioctyl sodium sulfosuccinate, sodium myreth sulfate, perfluorooctanesulfonate, alkyl ether phosphates; (iii) non-ionic surfactants such as alkylphenol ethoxylates (TritonX-100), fatty alcohol ethoxylates (octaethylene glycol monododecyl ether, cocamide diethanolamine, poloxamers, glycerolmonostearate, fatty acid esters of sorbitol (sorbitan monolaurate, Tween 80, Tween 20; and (iv) zwitterionic surfactants such as cocamidopropyl hydroxysultaine, and 3-[(3-Cholamidopropyl)dimethylammonio]-1-propanesulfonate (CHAPS). In other embodiments, the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, phosphatidylcholine, polyglycerol polyricinoleate, siloxanes, cetyl polyethylene glycol/polypropylene glycol-10/1 dimethicone triglyceride, bis-polyethylene glycol/polypropylene glycol-14/14 dimethicone, bis-(glyceryl/lauryl) glyceryl lauryl dimethicone (&) caprylic/capric triglyceride, cetyl polyethylene glycol/polypropylene glycol-10/1 dimethicone, phospholipids, or a combination thereof. In certain embodiments, the surfactant is polysorbate, docusate or lecithin. In certain other embodiments, the surfactant is polysorbate 20, polysorbate 60, or polysorbate 80. In still other embodiments, the surfactant is polysorbate 20 or polysorbate 80, e.g., Tween 20, Tween 60, Tween 80. In certain preferred embodiments, the fatty acid ester of sorbitol is a sorbitan ester, e.g., span 20, span 40, span 60, or span 80. In other preferred embodiments, the surfactant is an ionic surfactant. In preferred embodiments, the surfactant is polysorbate 80.

In some embodiments, the particles have greater than about 60% therapeutic biologic by weight after removing the first liquid, second liquid, and third liquid, e.g., greater than about 65, 70, 75, 80, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% therapeutic biologic by weight after removing the first liquid, second liquid, and third liquid. In other embodiments, the particles have greater than about 90% therapeutic biologic by weight after removing the first liquid, second liquid, and third liquid. In certain embodiments, the particles have greater than about 95% therapeutic biologic by weight after removing the first liquid, second liquid, and third liquid. In still other embodiments, the particles have greater than about 98% therapeutic biologic by weight after removing the first liquid, second liquid, and third liquid. In certain preferred embodiments, the particles have greater than about 99% therapeutic biologic by weight after removing the first liquid, second liquid, and third liquid.

In certain embodiments, the particles include a loading of therapeutic biologic from about 1 to about 100 wt % after removing the first liquid, second liquid, and third liquid, e.g., from about 50 to about 100 wt %, from about 75 to about 100 wt %, from about 90 to about 100 wt %, from about 95 to about 100 wt %, from about 99 to about 100 wt %, or from about 99.9 to about 100 wt % after removing the first liquid, second liquid, and third liquid. At these loadings the therapeutic biologic retains from about 0.5 to about 1.0 activity during particle formation, e.g., from about 0.75 to about 1.0 activity, from about 0.9 to about 1.0 activity, from about 0.95 to about 1.0 activity, from about 0.99 to about 1.0 activity, or from about 0.999 to about 1.0 activity. This includes the activity retained through primary desiccation (i.e., desiccation utilizing a third liquid) and, in some cases, secondary desiccation post processing.

In other embodiments, the particles have less than 10% aggregation or less than 10% fragmentation of the therapeutic biologic after removing the first liquid, second liquid, and third liquid, e.g., less than about 9, 8, 7, 6, 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% of the therapeutic biologic after removing the first liquid, second liquid, and third liquid. In certain embodiments, the particles have about 3% to about 1% aggregation of the therapeutic biologic after removing the first liquid, second liquid, and third liquid. In certain other embodiments, the particles have about 1% to about 0.5% aggregation of the therapeutic biologic after removing the first liquid, second liquid, and third liquid. In preferred embodiments, the particles are substantially free from any aggregation of the therapeutic biologic after removing the first liquid, second liquid, and third liquid. In still other embodiments, the particles have less than about 1% fragmentation of the therapeutic biologic after removing the first liquid, second liquid, and third liquid. In certain preferred embodiments, the particles are substantially free from any fragmentation of the therapeutic biologic after removing the first liquid, second liquid, and third liquid. Suitable methods for measuring aggregation and fragmentation of a biologic can be accomplished by using size-exclusion chromatography (SEC).

In some embodiments, the process of particle formation provides less than a 50% change in charge variants in the population of a therapeutic biologic after removing the first liquid, second liquid, and third liquid, e.g., an antibody, (e.g., less than 40, 30, 20, 10, 8, 5, 4, 3, or 1% after removing the first liquid, second liquid, and third liquid) as compared to the therapeutic biologic prior to particle formation. Charge variants may be acidic, basic, or neutral, and the variation may be caused post-translation modifications at terminal amino acids, such as asparagine deamidation or lysine glycation. For example, charge variants include the loss of a positive charge by the loss of a C-terminal lysine residue, covalent bonding of the amine portions of two lysine residues by reducing sugars, or the conversion of an N-terminal amine to a neutral amide by the cyclization of N-terminal glutamines. Negative charges on proteins, e.g., antibodies, can appear by the conversion of asparagine residues to aspartic acid and/or isoaspartic residues via a deamidation reaction. Exemplary methods of measuring charge variants include cation exchange chromatography (CIEX), where the variants are quantified by dividing the area under the peak corresponding to the variant, e.g., acidic, basic, or neutral population by the cumulative area contained beneath all peaks in the sample spectrum. Changes in charge variant population percentage between two samples, e.g., Sample A and Sample B, are computed as the numerical difference in the respective population variant percentages, i.e., by subtracting the specific variant, e.g., acidic, percentage of Sample B from the specific variant, e.g., acidic, percentage of Sample A, or vice versa. In certain embodiments, the analysis may be extended similarly for all variants within a population.

In certain embodiments, the particles have less than about 50% change in charge variants of the therapeutic biologic after removing the first liquid, second liquid, and third liquid, e.g., less than about 45, 40, 35, 30, 25, 20, 15, 10, 9, 8, 7, 6, 5, 4, 3, 2, 1, 0.5, 0.1% after removing the first liquid, second liquid, and third liquid, compared to the starting biologic prior to particle formation. In preferred embodiments, the particles are substantially free from any change in charge variants of the therapeutic biologic after removing the first liquid, second liquid, and third liquid, compared to the starting biologic prior to particle formation. Suitable methods for measuring a change in charge variants of a biologic can be accomplished by using cation exchange chromatography (CIEX).

In some embodiments, the particles have a surfactant content of less than about 10% by mass after removing the first liquid, second liquid, and third liquid, e.g., less than about 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, 0.001% by mass after removing the first liquid, second liquid, and third liquid. In other embodiments, the particles have a surfactant content of less than about 5% by mass after removing the first liquid, second liquid, and third liquid. In certain embodiments, the particles have a surfactant content of less than about 3% by mass after removing the first liquid, second liquid, and third liquid. In still other embodiments, the particles have a surfactant content of less than about 0.1% by mass after removing the first liquid, second liquid, and third liquid. In certain other embodiments, the particles have a surfactant content of less than about 0.01% by mass after removing the first liquid, second liquid, and third liquid. In some embodiments, the particles have a surfactant content of less than about 0.001% by mass after removing the first liquid, second liquid, and third liquid. In preferred embodiments, the particles have a surfactant content of less than about 1% by mass after removing the first liquid, second liquid, and third liquid. In certain preferred embodiments, the particles are substantially free from any surfactant content after removing the first liquid, second liquid, and third liquid.

In other embodiments, the surfactant content of the particles is from 0 to 10 wt % after removing the first liquid, second liquid, and third liquid, e.g., from 0 to 5 wt %, from 0 to 3 wt %, from 0 to 2 wt %, from 0 to 1 wt %, from 0 to 0.5 wt %, from 0 to 0.2 wt %, from 0 to 0.1 wt %, from 0 to 0.01 wt %, or from 0 to 0.001 wt % after removing the first liquid, second liquid, and third liquid. Exemplary methods of measuring the surfactant content include reconstitution of the particles in an appropriate medium, e.g., deionized water, and subsequent analysis of the reconstituted solution through liquid chromatography. The chromatographic technique may include the use of a charged aerosol detector (CAD) or an evaporative light scattering detector (ELSD).

In some embodiments, the residual first liquid, second liquid, and third liquid content remaining in the particles are less than about 3% by mass after removing the first liquid, second liquid, and third liquid, e.g., less than about 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% by mass remaining after removing the first liquid, second liquid, and third liquid. In other embodiments, the particles have less than about 2% of residual first liquid, second liquid, and third liquid by mass remaining after removing the first liquid, second liquid, and third liquid. In still other embodiments, the particles have less than about 1% of residual first liquid, second liquid, and third liquid by mass remaining after removing the first liquid, second liquid, and third liquid. In certain embodiments, the particles have less than about 0.1% of residual first liquid, second liquid, and third liquid by mass remaining after removing the first liquid, second liquid, and third liquid. In certain preferred embodiments, the particles have less than about 0.01% of residual first liquid, second liquid, and third liquid by mass remaining after removing the first liquid, second liquid, and third liquid. In preferred embodiments, the particles are substantially free from any residual first liquid, second liquid, and third liquid by mass after removing the first liquid, second liquid, and third liquid.

In other embodiments, the particles have about 1% to about 7% of residual first liquid, second liquid, and third liquid by mass remaining after removing the first liquid, second liquid, and third liquid. In still other embodiments, the particles have about 1% to about 5% of residual first liquid, second liquid, and third liquid by mass remaining after removing the first liquid, second liquid, and third liquid. In certain embodiments, the particles have about 1% to about 3% of residual first liquid, second liquid, and third liquid by mass remaining after removing the first liquid, second liquid, and third liquid. In preferred embodiments, the particles are substantially free from any residual first liquid, second liquid, and third liquid by mass after removing the first liquid, second liquid, and third liquid. Exemplary methods for the measurement of residual first liquid, second liquid, and third liquid content include chemical titration methods, e.g., Karl Fischer titration involving an oven. A variety of solvents, including water, may also be measured using weight loss methods involving thermal excitation. Exemplary methods include Thermogravimetric Analysis with Infrared Spectroscopy (TGA-IR) or Gas Chromatography Flame Ionization Detector Mass Spectrometry (GC-FID/MS).

In some embodiments, the residual moisture or solvent content of the particle is less than about 7% by weight after removing the first liquid, second liquid, and third liquid, e.g., less than about 6, 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% by weight after removing the first liquid, second liquid, and third liquid. In other embodiments, the particle has less than about 7% residual moisture by mass after removing the first liquid, second liquid, and third liquid. In still other embodiments, the particle has less than about 5% residual moisture by mass after removing the first liquid, second liquid, and third liquid. In certain embodiments, the particle has less than about 3% residual moisture by mass after removing the first liquid, second liquid, and third liquid. In certain preferred embodiments, the particle has less than about 2% residual moisture by mass after removing the first liquid, second liquid, and third liquid. In preferred embodiments, the particle has less than about 1% residual moisture by mass after removing the first liquid, second liquid, and third liquid. In some embodiments, the particle has less than about 0.1% residual moisture by mass after removing the first liquid, second liquid, and third liquid. In other embodiments, the particle has less than about 0.01% residual moisture by mass after removing the first liquid, second liquid, and third liquid. Exemplary methods for the measurement of moisture content include chemical titration methods, e.g., Karl Fischer titration involving an oven. A variety of solvents, including water, may also be measured using weight loss methods involving thermal excitation. Exemplary methods include Thermogravimetric Analysis with Infrared Spectroscopy (TGA-IR) or Gas Chromatography Flame Ionization Detector Mass Spectrometry (GC-FID/MS).

In other embodiments, the particle has about 1% to about 7% residual moisture by mass after removing the first liquid, second liquid, and third liquid. In still some embodiments, the particle has about 1% to about 5% residual moisture by mass after removing the first liquid, second liquid, and third liquid. In certain embodiments, the particle has about 1% to about 3% residual moisture by mass after removing the first liquid, second liquid, and third liquid. In preferred embodiments, the particle is substantially free from any residual moisture by mass after removing the first liquid, second liquid, and third liquid. In some embodiments, the particles are stable for at least one month. In still other embodiments, the particles are stable for at least two months. In certain other embodiments, the particles are stable for at least three months. In certain preferred embodiments, the particles are stable for at least three months at 40° C.

As used herein, the term “dispersity index” (DI) is a parameter characterizing the degree of non-uniformity of a size distribution of particles. The term “polydispersity index” (PDI) or “population dispersity” may be used interchangeably, is a parameter characterizing the width of the particle size distribution within a given sample. The numerical value of PDI ranges from 0.0 (for a perfectly uniform sample with respect to the particle size) to 1.0 and greater (for a highly polydisperse sample with multiple particle size populations). As the value decreases, the particles have more narrowly distributed particle sizes, and greater homogeneity of the plurality of particles. Particle diameter may be collected using microscopy (FlowCAM, SEM) as well as laser diffraction. The polydispersity index (PDI), “population dispersity” or “span”, can also mean a value that indicates the breadth of the particle size distribution. Particle size distribution are reported by D10, D50, D90, and the mean particle size in μm, with the values representing the percentage of particles that are smaller than the indicated D-number, e.g. the D10 particle size is the particle diameter at which 10% of the mass is composed of particles with a diameter less than this value, the D50 particle size is the particle diameter at which 50% of the mass is composed of particles with a diameter less than this value and the D90 particle size is the particle diameter at which 90% of the mass is composed of particles with a diameter less than this value. The D10, D50, and D90 particle size distribution is measured using a laser diffractometer, available from Horiba Corporation. It is worthy to note that such data, as described herein, indicates that the particle size distribution does not significantly decrease in size over time.

In other embodiments described herein, the particles have a polydispersity index from about 0.002 to about 1.000, e.g., from about 0.003, 0.004, 0.005, 0.006, 0.007, 0.008, 0.009, 0.010, 0.020, 0.030, 0.040, 0.050, 0.060, 0.070, 0.080, 0.090, 0.100, 0.200, 0.300, 0.400, 0.500, 0.600, 0.700, 0.800, 0.900 to about 1.000. In certain embodiments, the particles have a polydispersity index from about 0.002 to about 0.900. In certain preferred embodiments, the particles have a polydispersity index from about 0.100 to about 0.300.

In certain embodiments, the particles may include one or more therapeutic biologics. In other embodiments, the particles can have diameters from about 0.1 to about 1000 μm after removing the first liquid, second liquid, and third liquid, e.g., about 0.1 to about 90 μm, about 90 to about 230 μm, or about 0.1 to about 1 μm after removing the first liquid, second liquid, and third liquid. In still other embodiments, the particles can have a size dispersity from about 0 to about 0.9 after removing the first liquid, second liquid, and third liquid, e.g., from about 0 to about 0.7, from about 0 to about 0.5, or from about 0 to about 0.2 after removing the first liquid, second liquid, and third liquid. Methods of measuring the particle size and distribution include imaging flow cytometry, laser diffraction, and image analysis of scanning electron micrographs of the particles in which an average spherical radius or diameter can be calculated on the basis of the cross-sectional areas of the particles projected onto the plane of the image. In certain other embodiments of the disclosure, the particle may have a diameter of about 0.1 to about 1000 μm, a skeletal density of about 1.00 to about 6.00 g/cm3, and a glass transition temperature of about 0 to about 250° C., after removing the first liquid, second liquid, and third liquid.

The particles comprising at least one therapeutic biologic described herein, can be prepared in a number of ways, as well as any methods of forming the particles disclosed in, for example, PCT/US2017/063150, PCT/US2018/043774, PCT/US2019/033875, PCT/US20/15957, and PCT/US20/050508 each of which is hereby incorporated by reference in its entirety.

While each of the elements of the present disclosure is described herein, as containing multiple embodiments, it should be understood that, unless indicated otherwise, each of the embodiments of a given element of the present disclosure is capable of being used with each of the embodiments of the other elements of the present disclosure and each such use is intended to form a distinct embodiment of the present disclosure.

It will be understood by one of ordinary skill in the relevant arts that other suitable modifications and adaptations to the compositions and methods described herein are readily apparent from the description of the disclosure contained herein, in view of information known to the ordinarily skilled artisan, and may be made without departing from the scope of the disclosure or any embodiment thereof.

### Pharmaceutical Compositions

In certain embodiments according to the disclosure as described herein, a composition comprising a plurality of particles can have improved stability of the therapeutic biologic compared to an aqueous composition comprising the therapeutic biologic in monomeric form.

In some embodiments, the disclosure provides a composition containing a plurality of particles that include a therapeutic biologic, e.g., an antibody, bovine serum albumin (BSA), or human serum albumin (HSA), where the storage stability of the therapeutic biologic in the particles is improved with respect to the storage stability of the therapeutic biologic in the feed solution. In other embodiments, storage conditions are defined by time (e.g., more than about 2 years, more than about 1 year, more than about 6 months, more than about 3 months, more than about 1 month, or more than about 1 week) and temperature (e.g., about −80° C. to about 100° C., about −80° C. to about 60° C., about −20° C. to about 60° C., about 4 to about 60° C.), among potentially other variables. In still other embodiments, the storage time is about 3 days, about 7 days, about 30 days, about 90 days, about 180 days, about 1 year, or about 2 years. In certain embodiments, this temperature is about −80° C., about −40° C., about −20° C., about 4° C., about 25° C., about 40° C., or about 40 to about 60° C.

The phrase “pharmaceutically acceptable” is employed herein, to refer to those therapeutic biologics, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The term “pharmaceutically acceptable” can refer to particles and compositions comprising a plurality of particles that do not produce an adverse, allergic, or other untoward reaction when administered to a mammal, such as a human, as appropriate. The preparation of a pharmaceutical composition comprising an antibody or additional active ingredient will be known to those of skill in the art in light of the present disclosure. Moreover, for mammal (e.g., human) administration, it will be understood that preparations should meet sterility, pyrogenicity, general safety, and purity standards as required by FDA Office of Biological Standards.

The phrase “pharmaceutically acceptable liquid” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters. In certain preferred embodiments, the plurality of particles is suspended in a pharmaceutically acceptable liquid. In preferred embodiments, the liquid is a pharmaceutically acceptable liquid.

A pharmaceutical composition (formulation) as described herein, can be administered to a subject by any of a number of routes of administration including, for example, parenterally (including intramuscularly, intravenously, subcutaneously or intrathecally as, for example, a sterile solution or suspension); intraperitoneally; or subcutaneously. In certain embodiments, a composition may be simply suspended in a non-aqueous liquid carrier. Details of appropriate routes of administration and compositions suitable for same can be found in, for example, U.S. Pat. Nos. 6,110,973; 5,763,493; 5,731,000; 5,541,231; 5,427,798; 5,358,970 and 4,172,896, as well as in patents cited therein. The term “suspension formulation” refers to a liquid formulation including solid particles disposed within a carrier liquid in which they are not soluble on an appropriate timescale. The particles may settle over time, i.e., the physical stability of the suspension is not indefinite, but may be re-suspended using a form of agitation or excitation.

A “therapeutic amount” refers to an amount of a therapeutic biologic required to produce the desired effect. As used herein, the terms “treat,” “treated,” and “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

In some embodiments, insoluble particulate matter with characteristic sizes greater than or equal to about 100 μm that persist upon dissolution in an aqueous liquid are referred to as Visible Particles (VP). In preferred embodiments of the disclosure described herein, the composition is substantially free of Visible Particles (VP). In certain preferred embodiments, the aqueous liquid is water, aqueous buffer or a physiologically relevant aqueous liquid. In other embodiments, insoluble particulate matter which is visible to the naked eye under prescribed lighting conditions persist upon reconstitution of the particles of the disclosure into a liquid pharmaceutical composition. Insoluble particulates of this type, is sometimes referred to as Visible Particles (VPs), and are typically greater than about 100 μm in size. VPs are present in quantities from about 0 to about 1 per about 1 mL, e.g., from about 0 to about 0.01 per about 1 mL, from about 0 to about 0.001 per about 1 mL, or about 0 to about 0.0001 per about 1 mL. Exemplary methods of measuring VPs include analysis of the therapeutic biologic by visual inspection against and black and white background for 5 seconds under illumination of about 2000 and about 3750 lux in accordance with USP <790> after reconstitution and dilution of the therapeutic biologic to a standard concentration, e.g., about 100 mg/mL or about 1 mg/mL. In some embodiments, fewer than 65 samples in 10,000 (0.65%) are rejected on the basis of USP <790>. Alternate inspection strategies light-obscuration, automated optical imaging systems, or X-ray imaging in accordance with USP <1790>.

In other embodiments, insoluble particulate matter with characteristic sizes from about 1 μm to about 100 μm that persist upon dissolution in an aqueous liquid are referred to as Subvisible Particles (SvPs). SvPs are present in quantities from about 0 to 100,000,000 per about 1 mL, e.g., from about 0 to about 10,000,000 per about 1 mL, from about 0 to about 1,000,000 per about 1 mL, from about 0 to about 500,000 per about 1 mL, from about 0 to about 100,000 per about 1 mL, from about 0 to about 50,000 per about 1 mL, from about 0 to about 10,000 per about 1 mL, from about 0 to about 6,000 per about 1 mL, from about 0 to about 1,000 per about 1 mL, from about 0 to about 600 per about 1 mL, from about 0 to about 250 per about 1 mL, from about 0 to about 100 per about 1 mL, from about 0 to about 60 per about 1 mL, or from about 0 to about 10 per about 1 mL. In other embodiments, the count of particles with characteristic size greater than or equal to 10 μm is from about 0 to about 6,000 per about 1 mL, e.g., from about 0 to about 1,000 per about 1 mL, from about 0 to about 100 per about 1 mL, from about 0 to about 10 per about 1 mL, from about 0 to about 5 per about 1 mL, from about 0 to about 3 per about 1 mL, or from about 0 to about 1 per about 1 mL. In certain embodiments, the count of particles with characteristic size greater than or equal to 25 μm is from about 0 to about 600 per about 1 mL, e.g., from about 0 to about 100 per about 1 mL, from about 0 to about 10 per about 1 mL, from about 0 to about 3 per about 1 mL, from about 0 to about 1 per about 1 mL, from about 0 to about 0.5 per about 1 mL, or from about 0 to about 0.1 per about 1 mL. Exemplary methods of measuring SvPs include analysis of the therapeutic biologic with a Coulter Counter, HIAC Royco, or micro-flow imaging system after reconstitution and dilution of the therapeutic biologic to a standard concentration, e.g., about 100 mg/mL or about 1 mg/mL. In still other embodiments, the composition has a concentration of insoluble subvisible particles of about 0 per about 1 mL to about 100,000,000 per about 1 mL of greater than about 10 μm particles upon dissolution in an aqueous liquid. In certain embodiments, the composition has a concentration of insoluble subvisible particles of about 0 per about 1 mL to about 6000 per about 1 mL of greater than about 10 μm particles upon dissolution in an aqueous liquid. In preferred embodiments, the composition has a concentration of insoluble subvisible particles of about 0 per about 1 mL to about 600 per about 1 mL of greater than about 25 μm particles upon dissolution in an aqueous liquid. In certain preferred embodiments, the composition is substantially free of insoluble subvisible particles upon dissolution in an aqueous liquid. In preferred embodiments, the aqueous liquid is water, aqueous buffer or a physiologically relevant aqueous liquid.

In some embodiments, insoluble particulate matter with characteristic sizes from about 100 nm to about 1 μm that persist upon dissolution in an aqueous liquid are referred to as submicron particles (SMP) and sometimes known as nanoparticles. Quantitatively, SMPs are present in quantities from about 0 to 5×10¹² per about 1 mL, e.g., from about 0 to about 0.5×10¹² per about 1 mL, from about 0 to about 50×10⁹ per about 1 mL, from about 0 to about 10×10⁹ per about 1 mL, from about 0 to about 5×10⁹ per about 1 mL, from about 0 to about 0.5×10⁹ per about 1 mL, from about 0 to about 50×10⁶ per about 1 mL, from about 0 to about 1×10⁶ per about 1 mL, from about 0 to about 500,000 per about 1 mL, from about 0 to about 200,000 per about 1 mL, from about 0 to about 100,000 per about 1 mL, from about 0 to about 10,000 per about 1 mL, from about 0 to about 5000 per about 1 mL, or from about 0 to about 1000 per about 1 mL. Exemplary methods of measuring SMPs quantitatively include analysis of the therapeutic biologic with a NanoSight, asymmetric field flow fractionation coupled to a multi-angle laser light scattering (AF4 MALS), Dynamic Light Scattering (DLS), or any other nano-particle tracking device known in the art, after reconstitution and dilution of the therapeutic biologic to a standard concentration, e.g., about 100 mg/mL, about 1 mg/mL, or about 1 μg/mL. Qualitatively, SMPs are within a range comparable to the starting monomeric therapeutic biologic solution. In preferred embodiments, the composition is substantially free of submicron particles (SMP) upon dissolution in an aqueous liquid. In certain preferred embodiments, the aqueous liquid is water, aqueous buffer or a physiologically relevant aqueous liquid. Qualitatively, as described herein, SMPs are within a range comparable to the feed solution.

In certain embodiments, the pharmaceutical composition (formulation) includes insoluble particulate matter smaller than or equal to 1 μm. The pharmaceutical composition can have a concentration of insoluble particles with a characteristic size greater than or equal to about 100 nm is about 1 to 5×10′² per about 1 mL in suspension, or have a concentration of insoluble particles with a characteristic size less than or equal to about 1 μm is about 1 to 5×10′² per about 1 mL in suspension. In still other embodiments, the pharmaceutical composition of particles may include insoluble particulate matter larger than or equal to about 1 μm in size. In certain other embodiments, the number of insoluble particles is from about 0 to about 100,000,000 per about 1 mL, e.g., less than about 10,000,000, 1,000,000, 100,000, 10,000, 1000, 100, 10, or about 1 per about 1 mL. For example, the number of insoluble particles greater than about 10 μm is from about 0 to about 6,000 per about 1 mL, e.g., less than about 5,000, about 4,000, about 3,000, about 2,000, about 1,000, about 500, about 100, about 10, or about 1 per about 1 mL, and/or the number of insoluble particles greater than about 25 μm is from about 0 to about 600 per about 1 mL, e.g., less than about 500, about 400, about 300, about 200, about 100, about 50, about 10, or about 1 about 1 per about 1 mL.

In some embodiments, the disclosure provides a pharmaceutical composition, e.g., a suspension or dried form, containing a plurality of particles that include a therapeutic biologic, e.g., an antibody. The composition preferably has a concentration of insoluble particles, e.g., SvPs, of about 0 and about 100,000,000 per about 1 mL in suspension or upon reconstitution. In other embodiments, the concentration of insoluble particles is of about 0 and about 1,000,000 per about 1 mL in suspension or upon reconstitution. In still other embodiments, the concentration of insoluble particles is of about 0 and about 10,000 per about 1 mL in suspension or upon reconstitution. In certain other embodiments, the concentration of insoluble particles with a characteristic size greater than or equal to about 10 μm is of about 0 to about 6,000 per about 1 mL in suspension or upon reconstitution. In certain embodiments, the concentration of insoluble particles with a characteristic size greater than or equal to about 25 μm is of about 0 to about 600 per about 1 mL in suspension or upon reconstitution.

In other embodiments, after dissolution or reconstitution of the particles following storage, SvPs are present in quantities from about 0 to about 100,000,000 per about 1 mL, e.g., from about 0 to about 10,000,000 per about 1 mL, from about 0 to about 1,000,000 per about 1 mL, from about 0 to about 500,000 per about 1 mL, from about 0 to about 100,000 per about 1 mL, from about 0 to about 50,000 per about 1 mL, from about 0 to about 10,000 per about 1 mL, from about 0 to about 6,000 per about 1 mL, from about 0 to about 1,000 per about 1 mL, from about 0 to about 600 per about 1 mL, from about 0 to about 250 per about 1 mL, from about 0 to about 100 per about 1 mL, from about 0 to about 60 per about 1 mL, or from about 0 to about 10 per about 1 mL. In some embodiments, the count of particles with characteristic size greater than or equal to about 10 μm is from about 0 to about 6,000 per about 1 mL, e.g., from about 0 to about 1,000 per about 1 mL, from about 0 to about 100 per about 1 mL, from about 0 to about 10 per about 1 mL, from about 0 to about 5 per about 1 mL, from about 0 to about 3 per about 1 mL, or from about 0 to about 1 per about 1 mL. In certain embodiments, the count of particles with characteristic size greater than or equal to about 25 μm is from about 0 to about 600 per about 1 mL, e.g., from about 0 to about 100 per about 1 mL, from about 0 to about 10 per about 1 mL, from about 0 to about 3 per about 1 mL, from about 0 to about 1 per about 1 mL, from about 0 to about 0.5 per about 1 mL, or from about 0 to about 0.1 per about 1 mL. In still other embodiments, after dissolution or reconstitution of the particles following storage, the therapeutic biologic retains from about 0.5 to about 1.0 activity, e.g., from about 0.75 to about 1.0 activity, from about 0.9 to about 1.0 activity, from about 0.95 to about 1.0 activity, from about 0.99 to about 1.0 activity, or from about 0.999 to about 1.0 activity. In certain other embodiments, dissolution or reconstitution of the particles following storage provides less than about a 10% increase in aggregates of the therapeutic biologic, e.g., a protein, (e.g., less than about 8%, less than about 5%, less than about 4%, less than about 3%, less than about 1%, less than about 0.5%, or less than about 0.1%) as compared to the therapeutic biologic in the first liquid prior to processing. In certain embodiments, the dissolution or reconstitution of the particles after storage provides less than about a 10% increase in fragments of the therapeutic biologic, e.g., a protein, (e.g., less than about 8%, less than about 5%, less than about 4%, less than about 3%, less than about 1%, less than about 0.5%, or less than about 0.1%) as compared to the therapeutic biologic in the first liquid prior to processing. In some embodiments, the dissolution or reconstitution of the particles following storage provides less than about a 50% change in charge variants in the population of the therapeutic biologic, e.g., an antibody or an antibody fragment, (e.g., less than about 40, 30, 20, 10, 8, 5, 4, 3, or about 1%) as compared to therapeutic biologic prior to particle formation.

In still other embodiments, after dissolution or reconstitution of the particles following storage, SvPs are present in quantities from about 0 to about 100,000,000 per about 1 mL, e.g., from about 0 to about 10,000,000 per about 1 mL, from about 0 to about 1,000,000 per about 1 mL, from about 0 to about 500,000 per about 1 mL, from about 0 to about 100,000 per about 1 mL, from about 0 to about 50,000 per about 1 mL, from about 0 to about 10,000 per about 1 mL, from about 0 to about 6,000 per about 1 mL, from about 0 to about 1,000 per about 1 mL, from about 0 to about 600 per about 1 mL, from about 0 to about 250 per about 1 mL, from about 0 to about 100 per about 1 mL, from about 0 to about 60 per about 1 mL, or from about 0 to about 10 per about 1 mL. In certain embodiments, the count of particles with characteristic size greater than or equal to about 10 μm is from about 0 to about 6,000 per about 1 mL, e.g., from about 0 to about 1,000 per about 1 mL, from about 0 to about 100 per about 1 mL, from about 0 to about 10 per about 1 mL, from about 0 to about 5 per 1 mL, from about 0 to about 3 per about 1 mL, or from about 0 to about 1 per about 1 mL. In certain other embodiments, the count of particles with characteristic size greater than or equal to about 25 μm is from about 0 to about 600 per about 1 mL, e.g., from about 0 to about 100 per about 1 mL, from about 0 to about 10 per about 1 mL, from about 0 to about 3 per about 1 mL, from about 0 to about 1 per about 1 mL, from about 0 to about 0.5 per about 1 mL, or from about 0 to about 0.1 per about 1 mL. In some embodiments, dissolution or reconstitution of the particles following storage provides less than about a 10% increase in aggregates of the therapeutic biologic, e.g., an antibody, bovine serum albumin (BSA), or human serum albumin (HSA), (e.g., less than about 8%, less than about 5%, less than about 4%, less than about 3%, less than about 1%, less than about 0.5%, or less than about 0.1%) as compared to the therapeutic biologic in the first liquid prior to processing. In other embodiments, the dissolution or reconstitution of the particles after storage provides less than about a 10% increase in fragments of the therapeutic biologic, e.g., an antibody, bovine serum albumin (BSA), or human serum albumin (HSA), (e.g., less than about 8%, less than about 5%, less than about 4%, less than about 3%, less than about 1%, less than about 0.5%, or less than about 0.1%) as compared to the therapeutic biologic in the first liquid prior to processing. In certain other embodiments, the dissolution or reconstitution of the particles following storage provides less than about 50% change in charge variants in the population of a therapeutic biologic, e.g., an antibody, bovine serum albumin (BSA), or human serum albumin (HSA), e.g., less than about 40, about 30, about 20, about 10, about 8, about 5, about 4, about 3, or about 1%, as compared to the therapeutic biologic prior to particle formation.

In certain preferred embodiments, the present disclosure as described herein, concerns a highly concentrated composition comprising particles comprising at least one therapeutic biologic suspended in a low viscosity pharmaceutically acceptable liquid carrier, wherein the composition upon dissolution in water, buffers or other physiologically relevant aqueous liquids, e.g., biological fluids in the patients' body, have a substantially similar turbidity compared to a similar aqueous composition comprising monomeric therapeutic biologics. The term “turbidity” means the cloudiness or haziness of a fluid caused by individual particles that remain insoluble after dissolution at the desired concentration in water, buffer or other physiologically relevant aqueous liquids, e.g., biological fluids in the patients' body. As used herein, “physiologically relevant” conditions as may be encountered inside a mammal or human, can apply. The skilled person will be able to determine the set of conditions most appropriate for testing in accordance with the ultimate application of the compositions described herein. In some embodiments, the composition upon dissolution in an aqueous liquid has a substantially similar turbidity compared to an aqueous composition comprising monomeric therapeutic biologics. In preferred embodiments, the pharmaceutical composition upon dissolution in an aqueous liquid is substantially free of turbidity. In certain preferred embodiments, the aqueous liquid is water, aqueous buffer or a physiologically relevant aqueous liquid.

In some embodiments, the particles of the disclosure can be reconstituted into a liquid pharmaceutical composition to assess the turbidity or turbidance (USP <855>). Turbidity may be measured in units of FTU (Formazin Turbidity Units). This is achieved by comparing the turbidity of a sample with that of a formazine suspension. Turbidity may also be measured as Nephelometric Turbidity Units (NTU) where 1NTU=1FTU. In other embodiments, when 10 mg of particles are dissolved in 1 mL of liquid, turbidity can be of about 0 to about 4000 FTU, about 0 to about 1000 FTU, about 0 to about 500 FTU, about 0 to about 50 FTU, about 0 to about 20 FTU, about 0 to about 10 FTU, about 0 to about 5 FTU, about 0 to about 1 FTU, about 0 to about 0.1 FTU, or about 0 to about 0.01 FTU. In certain embodiments, the pharmaceutical composition has a turbidity of about 0 to about 4000 Formazin Turbidity Units (FTU). In certain other embodiments, the pharmaceutical composition upon dissolution in an aqueous liquid has a substantially similar turbidity compared to an aqueous composition comprising the therapeutic biologic in monomeric form. In preferred embodiments, the pharmaceutical composition upon dissolution in an aqueous liquid is substantially free of turbidity. In certain preferred embodiments, the aqueous liquid is water, aqueous buffer or a physiologically relevant aqueous liquid.

In other embodiments, the disclosure concerns highly concentrated compositions of low turbidity comprising a carbohydrate, a pH adjusting agent, a salt, a surfactant, a protein stabilizer, an emulsifier, an amino acid, and a plurality of particles comprising a therapeutic biologic, in a non-aqueous liquid carrier. In preferred embodiments, the disclosure concerns highly concentrated pharmaceutical compositions of low turbidity comprising trehalose, arginine hydrochloride, sodium succinate, succinic acid, citric acid, sodium citrate, histidine, histidine hydrochloride, sodium chloride, hydroxypropyl beta-cyclodextrin, sulfobutylether beta-cyclodextrin, polysorbate, or sorbitan monooleate, and a plurality of particles comprising an antibody, in ethyl oleate. In certain preferred embodiments, the pharmaceutical composition upon dissolution in water, aqueous buffer or any physiologically relevant aqueous liquid is substantially free of turbidity.

The particles comprising at least one therapeutic biologic described herein, can be used in a number of ways, as well as any methods for the delivery of the particles disclosed in, for example, PCT/US2017/063150, PCT/US2018/043774, PCT/US2019/033875, PCT/US20/15957, and PCT/US20/050508, each of which is hereby incorporated by reference in its entirety.

### Methods of the Disclosure

The methods described herein, are generally provided for forming particles, the method comprising: a) providing a first liquid comprising a therapeutic biologic and a solvent; b) contacting the first liquid with a second liquid, thereby forming liquid droplets comprising the therapeutic biologic; c) contacting the liquid droplets with a third liquid, thereby allowing the liquid droplets to dry; and d) removing the first liquid, second liquid, and third liquid, thereby forming the particles comprising a therapeutic biologic, wherein the particles comprise less than about 10% internal void spaces and the circularity of the particles is from about 0.80 to about 1.00 after removing the first liquid, second liquid, and third liquid. As disclosed herein, the therapeutic biologic is an antibody, bovine serum albumin (BSA), or human serum albumin (HSA). In certain embodiments, the therapeutic biologic has an activity per unit of about 0.5 to about 1.0. In certain preferred embodiments, the therapeutic biologic is an antibody. In preferred embodiments, the therapeutic biologic in the particles has an activity per unit of about 0.8 to about 1.0.

The methods as disclosed herein, allow the formation of an emulsion, e.g., droplets that resist aggregation and/or coalescence prior to drying. In some embodiments, the formation of an emulsion whereby the continuous phase does not dehydrate the droplets, allows for the control and optimization of the droplet size in the emulsion, which in turn determine the size of the particles. In other embodiments, the stabilization of droplets and emulsion can provide an optimized dehydration rate that allows the control of excipient and protein distribution in the particles.

In certain embodiments, the particle includes less than about 10% internal void spaces, less than about 5% internal void spaces, less than about 1% internal void spaces, less than about 0.1% internal void spaces, or less than about 0.01% internal void spaces after removing the first liquid, second liquid, and third liquid. In preferred embodiments, the particle is substantially free from any internal void spaces after removing the first liquid, second liquid, and third liquid.

In some embodiments, the circularity of the particle is from about 0.85 to about 1.00 after removing the first liquid, second liquid, and third liquid. In other embodiments, the circularity of the particle is from about 0.90 to about 1.00 after removing the first liquid, second liquid, and third liquid. In certain embodiments, the circularity of the particle is from about 0.95 to about 1.00 after removing the first liquid, second liquid, and third liquid. In preferred embodiments, the circularity of the particles is from about 0.98 to about 1.00 after removing the first liquid, second liquid, and third liquid. In certain preferred embodiments, the circularity of the particles is about 1.00 after removing the aqueous first liquid and organic second liquid.

### Droplets

Droplets as described herein, can be formed through any of several techniques as disclosed herein. These include membrane emulsification, homogenization, mechanical stirring, mechanical shaking, impinging jet mixing, ultra-sound, sonication, micro-channel emulsification, microsieve emulsification, capillary extrusion, static mixing, or a combination thereof. The terms “mixer” and “homogenizer” are used herein, interchangeably in the broadest sense. The term “droplet” or “droplets” or “drops” refer to a material that has a liquid outer surface. In some embodiments, the liquid droplets of step b) are formed by membrane emulsification, homogenization, mechanical stirring, mechanical shaking, impinging jet mixing, ultra-sound, sonication, micro-channel emulsification, microsieve emulsification, capillary extrusion, static mixing, or a combination thereof. In certain embodiments, the micro-channel emulsification is accomplished using a microfluidic chip based device. In other embodiments, the liquid droplets of step b) are formed by membrane emulsification, homogenization, impinging jet mixing, static mixing, or a combination thereof. In certain embodiments, the membrane emulsification is conducted by rotating membrane emulsification, cross-flow membrane emulsification, or a combination thereof. In still other embodiments, the homogenization is conducted by shear homogenization, pressure homogenization, rotor-stator homogenization, microfluidization, or a combination thereof. A person of ordinary skill in the field of this disclosure can readily assess the shear homogenization or pressure homogenization of the disclosed methods using routine and standard techniques for high or how shear homogenization, or high or low pressure homogenization. In certain other embodiments, the mechanical stirring is conducted by a turbulent stirred vessel, a magnetic stirring device, a mechanical stirring device, or a combination thereof. In certain preferred embodiments, the static mixing comprises laminar flow, turbulent flow, transition flow, or a combination thereof.

As used herein, the term “dispersion” refers to a droplet size suspension. Such dispersions may be stable due to the presence of components having both hydrophilic and hydrophobic sites, e.g., as in a surfactant or emulsifier. The terms “dispersed phase” (DP) and “continuous phase” (CP) are related to a dispersion system, in which a first liquid is dispersed within a second liquid. In such a dispersion system, the term “dispersed phase” (DP) or “feed solution” refers to a preparation of the therapeutic biologic in the solvent, either as a solution, a slurry, or some other liquid form, e.g., liquid droplets, dispersed in the continuous phase. The term “continuous phase” (CP) refers to a second liquid surrounding a first liquid, e.g., dispersed phase. As used herein, the term “emulsion” refers to a heterogeneous system consisting of a continuous phase and a non-continuous phase, e.g., the dispersed phase, capable of forming droplets in the continuous phase (CP). The term “emulsifier” refers to an agent that can reduce and/or eliminate the surface and the interfacial tension in a two-phase system. The emulsifier agent may possess both hydrophilic and lipophilic groups. The emulsifier may be considered to be either in the continuous phase (CP), dispersed phase (DP), or both. In some embodiments, the preparation contains excipients. In other embodiments, the preparation further contains a buffer. In certain embodiments, the preparation further contains a surfactant.

As described herein, in liquid-liquid or solid-liquid dispersions (emulsions or suspensions, respectively), the dispersed phase (DP) is present as discrete droplets or particles which are distributed throughout the continuous phase (CP).

In some embodiments, the solvent is an aqueous liquid, an organic solvent, an ionic liquid, a hydrogel, an ionogel, or a combination thereof. In other embodiments, the solvent is an aqueous liquid. In certain embodiments, the solvent is water, 0.9% saline, lactated Ringer's solution, a buffer, dextrose 5%, or a combination thereof. In preferred embodiments, the solvent is water. In other embodiments, the buffer is acetate buffer, histidine buffer, succinate buffer, HEPES buffer, tris buffer, carbonate buffer, citrate buffer, phosphate buffer, phosphate-buffered saline, glycine buffer, barbital buffer, cacodylate buffer, ammonium formate buffer, urea solution, or a combination thereof.

In other embodiments, the first liquid further comprises a carbohydrate, a pH adjusting agent, a salt, a chelator, a mineral, a polymer, a protein stabilizer, an emulsifier, an antiseptic, an amino acid, an antioxidant, a protein, an organic solvent, a paraben, a bactericide, a fungicide, a vitamin, a preservative, a nutrient media, an oligopeptide, a biologic excipient, a chemical excipient, a surfactant, or a combination thereof.

In certain embodiments, the carbohydrate is dextran, trehalose, sucrose, agarose, mannitol, lactose, sorbitol, maltose, or a combination thereof. In preferred embodiments, the carbohydrate is trehalose, cyclodextrins, hydroxypropyl beta-cyclodextrin, sulfobutylether beta-cyclodextrin, or a combination thereof.

In some embodiments, the pH adjusting agent is acetate, citrate, glutamate, glycinate, histidine, lactate, maleate, phosphate, succinate, tartrate, bicarbonate, aluminum hydroxide, phosphoric acid, hydrochloric acid, DL-lactic/glycolic acids, phosphorylethanolamine, tromethamine, imidazole, glyclyglycine, monosodium glutamate, sodium hydroxide, potassium hydroxide, or a combination thereof. In other embodiments, the pH adjusting agent is citrate, histidine, phosphate, succinate, sodium hydroxide, potassium hydroxide, or a combination thereof. In certain embodiments, the pH adjusting agent is hydrochloric acid or citric acid.

In other embodiments, the salt is sodium chloride, calcium chloride, potassium chloride, sodium hydroxide, stannous chloride, magnesium sulfate, sodium glucoheptonate, sodium pertechnetate, guanidine hydrochloride, potassium hydroxide, magnesium chloride, potassium nitrate, or a combination thereof. In preferred embodiments, the salt is sodium chloride.

In some embodiments, the chelator is disodium edetate, ethylenediaminetetraacetic acid, pentetic acid, or a combination thereof. In other embodiments, the mineral is calcium, zinc, titanium dioxide, or a combination thereof. In certain embodiments, the polymer is propyleneglycol, glucose star polymer, silicone polymer, polydimethylsiloxane, polyethylene glycol, carboxymethylcellulose, poly(glycolic acid), poly(lactic-co-glycolic acid), polylactic acid, polycaprolactone (PCL), polyvinylpyrrolidone (PVP), ficoll, dextran, or a combination thereof.

In certain embodiments, the protein stabilizer is trehalose, polyethylene glycol (PEG), polyoxamers, polyvinylpyrrolidone, polyacrylic acids, poly(vinyl) polymers, polyesters, polyaldehydes, tert-polymers, polyamino acids, hydroxyethyl starch, N-methyl-2-pyrrolidone, sorbitol, sucrose, mannitol, cyclodextrin, hydroxypropyl beta-cyclodextrin, sulfobutylether beta-cyclodextrin, or a combination thereof. In preferred embodiments, the protein stabilizer is trehalose, cyclodextrin, hydroxypropyl beta-cyclodextrin, sulfobutylether beta-cyclodextrin, or a combination thereof. In certain preferred embodiments, the PEG is PEG 200, PEG 300, PEG 3350, PEG 8000, PEG 10000, PEG 20000, or a combination thereof.

In some embodiments, the emulsifier is polysorbate 80, polysorbate 60, polysorbate 20, sorbitan monooleate, ethanolamine, polyoxyl 35 castor oil, poloxyl 40 hydrogenated castor oil, carbomer 1342, a corn oil-mono-di-triglyceride, a polyoxyethylated oleic glyceride, a poloxamer, or a combination thereof. In preferred embodiments, the fatty acid ester of sorbitol is a sorbitan ester, e.g., span 20, span 40, span 60, or span 80. In certain preferred embodiments, the emulsifier is polysorbate 80, sorbitan monooleate, or a combination thereof.

In other embodiments, the antiseptic is phenol, m-cresol, benzyl alcohol, 2-phenyloxyethanol, chlorobutanol, neomycin, benzethonium chloride, gluteraldehyde, beta-propiolactone, or a combination thereof.

In certain other embodiments, the amino acid is alanine, aspartic acid, cysteine, isoleucine, glutamic acid, leucine, methionine, phenylalanine, pyrrolysine, serine, selenocysteine, threonine, tryptophan, tyrosine, valine, asparagine, arginine, histidine, glycine, glutamine, proline, or a combination thereof. In certain preferred embodiments, the amino acid is arginine, histidine, proline, asparagine, or a combination thereof. In preferred embodiments, the amino acid is histidine.

In some embodiments, the antioxidant is glutathione, ascorbic acid, cysteine, N-acety-L-tryptophanate, tocopherol, histidine, methionine, or a combination thereof. In other embodiments, the protein is protamine, protamine sulfate, gelatin, or a combination thereof. In certain embodiments, the organic solvent is dimethyl sulfoxide, N-methyl-2-pyrrolidone, or a combination thereof. The paraben can be a parahydroxybenzoate. In still other embodiments, the bactericide is benzalkonium chloride (cationic surfactants), hypochlorites, peroxides, alcohols, phenolic compounds (e.g. carbolic acid), benzyl benzoate, or a combination thereof. In preferred embodiments, the bactericide is benzyl benzoate.

In other embodiments, the fungicide is acibenzolar, 2-phenylphenol, anilazine, carvone, natamycin, potassium azide, or a combination thereof. In preferred embodiments, the fungicide is benzyl benzoate. In certain embodiments, the vitamin is thiamine, riboflavin, niacin, pantothenic acid, biotin, vitamin B₆, vitamin B₁₂, folate, niacin, ascorbic acid, calciferols, retinols, quinones, or a combination thereof. In still other embodiments, the preservative is sodium nitrate, sulfur dioxide, potassium sorbate, sodium sorbate, sodium benzoate, benzoic acid, methyl hydroxybenzoate, thimerosal, parabens, formaldehyde, castor oil, or a combination thereof. In preferred embodiments, the preservative is methyl hydroxybenzoate, thimerosal, a paraben, formaldehyde, castor oil, or a combination thereof.

A number of nutrient media, preferably serum free, alone or in combination, may be used in the present disclosure, including commercially available media or other media well known in the art. In addition, serum-containing nutrient media may also be used in compositions according to the present disclosure, but the use of serum-containing media is less preferred because of the possibility that the serum may be contaminated with microbial agents and because the patient may develop immunological reactions to certain antigenic components contained in the serum.

In some embodiments, the oligopeptide is trileucine. In other embodiments, the biologic excipient are nucleic acids, oligonucleotides, antibodies or fragment thereof, amino acids, polyamino acids, peptides, proteins, cells, bacteria, gene therapeutics, genome engineering therapeutics, epigenome engineering therapeutics, hormones, nucleoproteins, glycoproteins, lipoproteins, exosomes, outer membrane vesicles, vaccines, viruses, bacteriophages, organelles, nutrient media, or a combination thereof. In certain embodiments, the chemical excipient are chemical drugs, contrast agents, dyes, magnetic particles, polymer beads, metal nanoparticles, metal microparticles, quantum dots, antioxidants, antibiotic agents, steroids, analgesics, local anesthetics, anti-inflammatory agents, parabens, anti-microbial agents, chemotherapeutic agents, vitamins, minerals, bactericides, antiseptics, or a combination thereof.

In other embodiments, the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, phosphatidylcholine, polyglycerol polyricinoleate, siloxanes, cetyl polyethylene glycol/polypropylene glycol-10/1 dimethicone triglyceride, bis-polyethylene glycol/polypropylene glycol-14/14 dimethicone, bis-(glyceryl/lauryl) glyceryl lauryl dimethicone (&) caprylic/capric triglyceride, cetyl polyethylene glycol/polypropylene glycol-10/1 dimethicone, phospholipids, or a combination thereof. In certain embodiments, the surfactant is polysorbate, docusate or lecithin. In certain other embodiments, the surfactant is polysorbate 20, polysorbate 60, or polysorbate 80. In still other embodiments, the surfactant is polysorbate 20 or polysorbate 80, e.g., Tween 20, Tween 60, Tween 80. In certain preferred embodiments, the fatty acid ester of sorbitol is a sorbitan ester, e.g., span 20, span 40, span 60, or span 80. In other preferred embodiments, the surfactant is an ionic surfactant. In preferred embodiments, the surfactant is polysorbate 80.

In some embodiments, the concentration of the therapeutic biologic in the first liquid as described herein, is about 10 mg/mL to about 650 mg/mL, e.g., about 20, 30, 50, 75, 100, 125, 150, 175, 200, 225, 250, 275, 300, 325, 350, 375, 400, 425, 450, 475, 500, 525, 550, 575, 600, 625 mg/mL to about 650 mg/mL; about 20 mg/mL to about 625 mg/mL, e.g., about 20, 30, 50, 75, 100, 125, 150, 175, 200, 225, 250, 275, 300, 325, 350, 375, 400, 425, 450, 475, 500, 525, 550, 575, 600 mg/mL to about 625 mg/mL; about 20 mg/mL to about 600 mg/mL, e.g., about 20, 30, 50, 75, 100, 125, 150, 175, 200, 225, 250, 275, 300, 325, 350, 375, 400, 425, 450, 475, 500, 525, 550, 575 mg/mL to about 600 mg/mL; about 20 mg/mL to about 575 mg/mL, e.g., about 20, 30, 50, 75, 100, 125, 150, 175, 200, 225, 250, 275, 300, 325, 350, 375, 400, 425, 450, 475, 500, 525, 550 mg/mL to about 575 mg/mL; about 20 mg/mL to about 550 mg/mL, e.g., about 20, 30, 50, 75, 100, 125, 150, 175, 200, 225, 250, 275, 300, 325, 350, 375, 400, 425, 450, 475, 500, 525 mg/mL to about 550 mg/mL; about 20 mg/mL to about 525 mg/mL, e.g., about 20, 30, 50, 75, 100, 125, 150, 175, 200, 225, 250, 275, 300, 325, 350, 375, 400, 425, 450, 475, 500 mg/mL to about 525 mg/mL; about 20 mg/mL to about 500 mg/mL, e.g., about 20, 30, 50, 75, 100, 125, 150, 175, 200, 225, 250, 275, 300, 325, 350, 375, 400, 425, 450, 475 mg/mL to about 500 mg/mL; about 20 mg/mL to about 475 mg/mL, e.g., about 20, 30, 50, 75, 100, 125, 150, 175, 200, 225, 250, 275, 300, 325, 350, 375, 400, 425, 450 mg/mL to about 475 mg/mL; about 20 mg/mL to about 450 mg/mL, e.g., about 20, 30, 50, 75, 100, 125, 150, 175, 200, 225, 250, 275, 300, 325, 350, 375, 400, 425 mg/mL to about 450 mg/mL; about 20 mg/mL to about 425 mg/mL, e.g., about 20, 30, 50, 75, 100, 125, 150, 175, 200, 225, 250, 275, 300, 325, 350, 375, 400 mg/mL to about 425 mg/mL; about 20 mg/mL to about 400 mg/mL, e.g., about 20, 30, 50, 75, 100, 125, 150, 175, 200, 225, 250, 275, 300, 325, 350, 375 mg/mL to about 400 mg/mL; about 20 mg/mL to about 375 mg/mL, e.g., about 20, 30, 50, 75, 100, 125, 150, 175, 200, 225, 250, 275, 300, 325, 350 mg/mL to about 375 mg/mL; about 20 mg/mL to about 350 mg/mL, e.g., about 20, 30, 50, 75, 100, 125, 150, 175, 200, 225, 250, 275, 300, 325 mg/mL to about 350 mg/mL; about 20 mg/mL to about 325 mg/mL, e.g., about 20, 30, 50, 75, 100, 125, 150, 175, 200, 225, 250, 275, 300 mg/mL to about 325 mg/mL; or about 20 mg/mL to about 300 mg/mL, e.g., about 20, 30, 50, 75, 100, 125, 150, 175, 200, 225, 250, 275 mg/mL to about 300 mg/mL. In other embodiments, the concentration of the therapeutic biologic in the first liquid is about 10 mg/mL to about 500 mg/mL. In certain embodiments, the concentration of the therapeutic biologic in the first liquid is about 10 mg/mL to about 100 mg/mL. In preferred embodiments, the concentration of the therapeutic biologic in the first liquid is about 20 mg/mL to about 100 mg/mL. In other embodiments of the disclosure, the concentration of the therapeutic biologic in the first liquid is from about 0.0001 mg/mL to about 1000 mg/mL, e.g., about 100 to about 800, about 200 to about 700, about 200 to about 600, or about 300 mg/mL to about 700 mg/mL. In still other embodiments, the particles have a mass loading of the therapeutic biologic from about 1% to about 100%.

In other embodiments, the first liquid has a viscosity of less than about 200 mPa·s, less than about 150 mPa·s, less than about 125 mPa·s, less than about 100 mPa·s, less than about 75 mPa·s, less than about 75 mPa·s, less than about 70 mPa·s, less than about 65 mPa·s, less than about 60 mPa·s, less than about 55 mPa·s, less than about 50 mPa·s, less than about 45 mPa·s, less than about 40 mPa·s, less than about 35 mPa·s, less than about 30 mPa·s, less than about 25 mPa·s, less than about 20 mPa·s, less than about 19 mPa·s, less than about 18 mPa·s, less than about 17 mPa·s, less than about 16 mPa·s, less than about 15 mPa·s, less than about 14 mPa·s, less than about 13 mPa·s, less than about 12 mPa·s, less than about 11 mPa·s, less than about 10 mPa·s, less than about 9.5 mPa·s, less than about 9 mPa·s, less than about 8.5 mPa·s, less than about 8 mPa·s, less than about 7.5 mPa·s, less than about 7 mPa·s, less than about 6.5 mPa·s, less than about 6 mPa·s, less than about 5.5 mPa·s, less than about 5 mPa·s, less than about 4.5 mPa·s, less than about 4 mPa·s, less than about 3.5 mPa·s, less than about 3 mPa·s, less than about 2.5 mPa·s, less than about 2 mPa·s, less than about 1.5 mPa·s, less than about 1 mPa·s, less than about 0.5 mPa·s, less than about 0.1 mPa·s, less than about 0.05 mPa·s, or less than about 0.01 mPa·s (one millipascal-second). In other embodiments, the first liquid has a viscosity of about 0.01 mPa·s to about 10,000 mPa·s, e.g., from about 0.01 mPa·s to about 1,000 mPa·s, from about 0.01 mPa·s to about 100 mPa·s, from about 0.01 mPa·s to about 50 mPa·s, from about 0.01 mPa·s to about 25 mPa·s, from about 0.01 mPa·s to about 10 mPa·s, from about 0.01 mPa·s to about 5 mPa·s, or from about 0.01 mPa·s to about 1 mPa·s. In certain embodiments, the first liquid has a viscosity that ranges from about 0.27 mPa·s to about 200 mPa·s, e.g., about 0.27 mPa·s to about 50 mPa·s, about 1 mPa·s to about 30 mPa·s, or about 20 mPa·s to about 50 mPa·s. In still other embodiments, the first liquid has a viscosity that ranges from about 0.27 mPa·s to about 200 mPa·s, e.g., about 0.27 mPa·s to about 100 mPa·s, about 0.27 mPa·s to about 50 mPa·s, about 0.27 mPa·s to about 30 mPa·s, about 1 mPa·s to about 20 mPa·s, or about 1 mPa·s to about 15 mPa·s. The term “viscosity” is used to describe the property of a fluid acting to resist shearing flow. For the purposes of the present disclosure, viscosity can be determined using a rheometer, e.g., AR-G2 Rheometer (TA Instruments, USA), fitted with a cone and plate (2°/40 mm) at 25° C. at a specified shear rate. In certain embodiments, the viscosity is measured at a shear rate in the Newtonian regime. The term “Newtonian regime” means a range of shear rates which are linearly proportional or nearly linearly proportional to the local strain rate at every point. In some embodiments, the viscosity is measured at a shear rate of about 100 s⁻¹ or greater, e.g., at about 1000 s⁻¹ or greater than about 1000 s⁻¹. Methods of controlling viscosity include temperature regulation and viscosity modifying additives. Mixtures of liquids may also be used to control viscosity. The units “mPa·s” and “cP” are used herein, interchangeably in the broadest sense.

In some embodiments, the first liquid has a viscosity from about 0.01 mPa·s to about 10,000 mPa·s. In other embodiments, the first liquid has a viscosity of less than about 100 mPa·s. In still other embodiments, the first liquid has a viscosity of less than about 10 mPa·s. In certain other embodiments, the first liquid has a viscosity of less than about 3 mPa·s. In preferred embodiments, the first liquid has a viscosity of less than about 0.9 mPa·s.

In other embodiments, the second liquid is an aqueous liquid, an organic solvent, an ionic liquid, a hydrogel, ionogel, protein stabilizer, or a combination thereof. In preferred embodiments, the second liquid is an organic solvent.

In some embodiments, the organic solvent is acetone, acetonitrile, acyclic alkanes (e.g., hexanes, heptane, pentane), amyl acetate, butanol, butyl acetate, chlorobenzene, chloroform, cumene, cyclohexane, 1,2-dichloroethene, dichloromethane, diethyl ether, dimethoxyethane, dimethylacetamide, dimethylformamide, dimethyl sulfoxide, 1,4-dioxane, ethanol, 2-ethoxyethanol, ethyl acetate, ethyl nitrate, ethyleneglycol, hydrazine, isopropanol, methanol, methyl acetate, 2-methyl-1-butanol, 2-methyl-1-propanol, methylbutyl ketone, methylcyclohexane, methylethyl ketone, methylpyrrolidone, methyl tert-butyl ether, nitromethane, propanol, propyl acetate, sulfolane, propyleneglycol, tetrahydrofuran, tetralin, toluene, 1,1,2-tricholoroethane, triethylamine, xylene, benzyl benzoate, ethyl lactate, dimethyl isosorbide, dimethyl sulfoxide, glycofurol, diglyme, methyl tert-butyl ether, polyethylene glycol, 2-pyrrolidone, tetrahydrofurfuryl alcohol, trigylcerides, octyl acetate, ethanol, butanol, octanol, decanol, diglyme, tocopherol, octa-fluoropropane, (perfluorohexyl)octane, n-acetyltryptophan, trigylcerides, triglycerides of the fractionated plant fatty acids C8 and C10, propylene glycol diesters of saturated plant fatty acids C8 and C10, ethyl laurate, methyl caprylate, methyl caprate, methyl myristate, methyl oleate, methyl linoleate, dimethyl adipate, dibutyl suberate, diethyl sebacate, ethyl macadamiate, trimethylolpropane triisosterate, isopropyl laurate, isopropyl myristate, diethyl succinate, polysorbate esters, ethanol amine, propanoic acid, triacetin, citral, anisole, anethol, benzaldehyde, linalool, caprolactone, phenol, thioglycerol, dimethylacetamide, ethyl formate, ethyl hexyl acetate, eugenol, clove bud oil, diethyl glycol monoether, dimethyl isosorbide, isopropyl acetate, methyl isobutyl ketone, methyl tert-butyl ether, N-methyl pyrrolidone, perfluorodecalin, 2-pyrrolidone, ethyl oleate, ethyl caprate, dibutyl adipate, hexanoic acid, octanoic acid, diethyl glycol monoether, gamma-butyrolactone, polyoxyl 40 hydrogenated castor oil, polyoxyl 35 castor oil, propylene carbonate, octanol, hexanol, sorbitan monooleate, n-acetyltryptophan, solketal, an alkyl acetate, an aryl acetate, an aryl alkyl acetate, tolyl acetate, benzyl acetate, polysorbate 80, phenethyl acetate, phenyl acetate, glycerol, or a combination thereof.

In other embodiments, the organic solvent is benzyl alcohol, benzyl benzoate, castor oil, coconut oil, corn oil, cottonseed oil, fish oil, grape seed oil, hazelnut oil, hydrogenated palm seed oil, olive oil, peanut oil, peppermint oil, safflower oil, sesame oil, soybean oil, sunflower oil, vegetable oil, walnut oil, polyethylene glycol, glycofurol, acetone, diglyme, dimethylacetamide, dimethyl isosorbide, dimethyl sulfoxide, ethanol, methyl acetate, ethyl acetate, propyl acetate, butyl acetate, ethyl ether, ethyl lactate, isopropyl acetate, methyl acetate, methyl isobutyl ketone, methyl tert-butyl ether, N-methyl pyrrolidone, perfluorodecalin, 2-pyrrolidone, trigylcerides, tetrahydrofurfuryl alcohol, triglycerides of the fractionated plant fatty acids C8 and C10 (e.g., MIGLYOL® 810 and MIGLOYL® 812N), propylene glycol diesters of saturated plant fatty acids C8 and C10 (e.g., MIGLYOL® 840), ethyl oleate, ethyl caprate, dibutyl adipate, fatty acid esters, hexanoic acid, octanoic acid, triacetin, diethyl glycol monoether, gamma-butyrolactone, eugenol, clove bud oil, citral, limonene, hexanes, heptane, or a combination thereof. In certain embodiments, the organic solvent is methylacetate, ethylacetate, propylacetate, butylacetate, amylacetate, 2-ethylhexylacetate, heptane, or a combination thereof. In preferred embodiments, the organic solvent is heptane.

In certain embodiments, the organic solvent is acetonitrile, chlorobenzene, chloroform, cyclohexane, cumene, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, methylisobutylketone, N-methylpyrrolidone, nitromethane, pyridine, sulfolane, tetrahydrofuran, tetralin, toluene, 1,1,2-trichloroethene, xylene, acetic acid, acetone, anisole, 1-butanol, 2-butanol, butylacetate, tert-butylmethyl ether, dimethyl sulfoxide, ethanol, ethylacetate, ethyl ether, ethyl formate, formic acid, heptane, isobutylacetate, isopropylacetate, methylacetate, 3-methyl-1-butanol, methylethyl ketone, 2-methyl-1-propanol, pentane, 1-pentanol, 1-propanol, 2-propanol, propylacetate, triethylamine, 1,1-diethoxypropane, 1,1-dimethoxymethane, 2,2-dimethoxypropane, isooctane, isopropyl ether, methylisopropyl ketone, methyltetrahydrofuran, petroleum ether, trichloroacetic acid, trifluoroacetic acid, decanol, 2-ethylhexylacetate, amylacetate, or a combination thereof.

In some embodiments, the second liquid further comprises a carbohydrate, a pH adjusting agent, a salt, a chelator, a mineral, a polymer, a protein stabilizer, an emulsifier, an antiseptic, an amino acid, an antioxidant, a protein, an organic solvent, a paraben, a bactericide, a fungicide, a vitamin, a preservative, a nutrient media, an oligopeptide, a biologic excipient, a chemical excipient, a surfactant, or a combination thereof. In preferred embodiments, the second liquid further comprises a surfactant.

In other embodiments, the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, phosphatidylcholine, polyglycerol polyricinoleate, siloxanes, cetyl polyethylene glycol/polypropylene glycol-10/1 dimethicone triglyceride, bis-polyethylene glycol/polypropylene glycol-14/14 dimethicone, bis-(glyceryl/lauryl) glyceryl lauryl dimethicone (&) caprylic/capric triglyceride, cetyl polyethylene glycol/polypropylene glycol-10/1 dimethicone, phospholipids, or a combination thereof. In certain embodiments, the surfactant is polysorbate, docusate or lecithin. In certain other embodiments, the surfactant is polysorbate 20, polysorbate 60, or polysorbate 80, e.g., Tween 20, Tween 60, Tween 80. In still other embodiments, the surfactant is polysorbate 20 or polysorbate 80. In certain preferred embodiments, the fatty acid ester of sorbitol is a sorbitan ester, e.g., span 20, span 40, span 60, or span 80. In other preferred embodiments, the surfactant is an ionic surfactant. In preferred embodiments, the surfactant is polysorbate 80.

In some embodiments, the second liquid has a viscosity of less than about 200 mPa·s, less than about 150 mPa·s, less than about 125 mPa·s, less than about 100 mPa·s, less than about 75 mPa·s, less than about 75 mPa·s, less than about 70 mPa·s, less than about 65 mPa·s, less than about 60 mPa·s, less than about 55 mPa·s, less than about 50 mPa·s, less than about 45 mPa·s, less than about 40 mPa·s, less than about 35 mPa·s, less than about 30 mPa·s, less than about 25 mPa·s, less than about 20 mPa·s, less than about 19 mPa·s, less than about 18 mPa·s, less than about 17 mPa·s, less than about 16 mPa·s, less than about 15 mPa·s, less than about 14 mPa·s, less than about 13 mPa·s, less than about 12 mPa·s, less than about 11 mPa·s, less than about 10 mPa·s, less than about 9.5 mPa·s, less than about 9 mPa·s, less than about 8.5 mPa·s, less than about 8 mPa·s, less than about 7.5 mPa·s, less than about 7 mPa·s, less than about 6.5 mPa·s, less than about 6 mPa·s, less than about 5.5 mPa·s, less than about 5 mPa·s, less than about 4.5 mPa·s, less than about 4 mPa·s, less than about 3.5 mPa·s, less than about 3 mPa·s, less than about 2.5 mPa·s, less than about 2 mPa·s, less than about 1.5 mPa·s, less than about 1 mPa·s, less than about 0.5 mPa·s, less than about 0.1 mPa·s, less than about 0.05 mPa·s, or less than about 0.01 mPa·s (one millipascal-second). In other embodiments, the second liquid has a viscosity of about 0.01 mPa·s to about 10,000 mPa·s, e.g., from about 0.01 mPa·s to about 1,000 mPa·s, from about 0.01 mPa·s to about 100 mPa·s, from about 0.01 mPa·s to about 50 mPa·s, from about 0.01 mPa·s to about 25 mPa·s, from about 0.01 mPa·s to about 10 mPa·s, from about 0.01 mPa·s to about 5 mPa·s, or from about 0.01 mPa·s to about 1 mPa·s. In certain embodiments, the second liquid has a viscosity that ranges from about 0.27 mPa·s to about 200 mPa·s, e.g., about 0.27 mPa·s to about 50 mPa·s, about 1 mPa·s to about 30 mPa·s, or about 20 mPa·s to about 50 mPa·s. In still other embodiments, the second liquid has a viscosity that ranges from about 0.27 mPa·s to about 200 mPa·s, e.g., about 0.27 mPa·s to about 100 mPa·s, about 0.27 mPa·s to about 50 mPa·s, about 0.27 mPa·s to about 30 mPa·s, about 1 mPa·s to about 20 mPa·s, or about 1 mPa·s to about 15 mPa·s. Methods of controlling viscosity include temperature regulation and viscosity modifying additives. Mixtures of liquids may also be used to control viscosity.

In other embodiments, the second liquid has a viscosity from about 0.01 mPa·s to about 10,000 mPa·s. In some embodiments, the second liquid has a viscosity of less than about 50 mPa·s. In still other embodiments, the second liquid has a viscosity of less than about 10 mPa·s. In certain other embodiments, the second liquid has a viscosity of less than about 5 mPa·s. In certain embodiments, the second liquid has a viscosity of less than about 2 mPa·s. In preferred embodiments, the second liquid has a viscosity of less than about 0.40 mPa·s.

In some embodiments, the liquid droplets of step b) are formed by membrane emulsification, homogenization, mechanical stirring, mechanical shaking, impinging jet mixing, ultra-sound, sonication, micro-channel emulsification, microsieve emulsification, capillary extrusion, static mixing, or a combination thereof. In certain embodiments, the micro-channel emulsification is accomplished using a microfluidic chip based device. In other embodiments, the liquid droplets of step b) are formed by membrane emulsification, homogenization, impinging jet mixing, static mixing, or a combination thereof. In certain embodiments, the membrane emulsification is conducted by rotating membrane emulsification, cross-flow membrane emulsification, or a combination thereof. In still other embodiments, the homogenization is conducted by shear homogenization, pressure homogenization, rotor-stator homogenization, microfluidization, or a combination thereof. A person of ordinary skill in the field of this disclosure can readily assess the shear homogenization or pressure homogenization of the disclosed methods using routine and standard techniques for high or how shear homogenization, or high or low pressure homogenization. In certain other embodiments, the mechanical stirring is conducted by a turbulent stirred vessel, a magnetic stirring device, a mechanical stirring device, or a combination thereof. In certain preferred embodiments, the static mixing comprises laminar flow, turbulent flow, transition flow, or a combination thereof.

In other embodiments, each of the further components in the first liquid, second liquid, and/or third liquid is independently, about 0.0001 to about 99% (w/v), e.g., about 0.0001 to about 90% (w/v), about 0.0001 to about 50% (w/v), about 0.0001 to about 10% (w/v), about 0.0001 to about 1% (w/v), or about 0.0001 to about 0.1% (w/v). In certain embodiments, the amount of additional components, i.e., excipient, present in the first liquid, second liquid, and/or third liquid, is as shown Table 1.

TABLE 1

| Excipient | Range 1 | Range 2 | Range 3 | Range 4 |
|---|---|---|---|---|
| Carbohydrate | 10-30% | 3-50% | 1-80% | 0.3-99% |
| pH adjusting | 0.5-5% | 0.2-40% | 0.05-70% | 0.01-99% |
| agent | | | | |
| Salt | 10-50% | 3-70% | 1-85% | 0.3-99% |
| Chelator | 0.01-1% | 0.003-40% | 0.001-80% | 0.0003-99% |
| Mineral | 10-50% | 3-70% | 1-80% | 0.3-99% |
| Polymer | 10-60% | 3-75% | 1-85% | 0.3-99% |
| Surfactant | .01-1% | 0.003-40% | 0.001-80% | 0.0003-99% |
| Amino acids | 10-25% | 3-50% | 1-85% | 0.3-99% |
| Oligopeptide | 10-25% | 3-50% | 1-85% | 0.3-99% |
| Biologic | 10-70% | 3-70% | 1-85% | 0.3-99% |
| Chemical | 10-50% | 3-70% | 1-85% | 0.3-99% |
| Antiseptic | .5-10% | 0.2-50% | 0.05-70% | 0.02-99% |
| Antioxidant | 0.01-1% | 0.003-40% | 0.001-80% | 0.0003-99% |
| Paraben | 0.01-5% | 0.005-10% | 0.001-50% | 0.001-99% |
| Bactericide | 0.01-5% | 0.005-10% | 0.001-50% | 0.001-99% |
| Fungicide | 0.01-5% | 0.005-10% | 0.001-50% | 0.001-99% |
| Vitamin | 1-50% | 1-70% | 0.1-85% | 0.01-99% |
| Preservative | 10-50% | 3-70% | 1-85% | 0.3-99% |
| Analgesic | 0.01-5% | 0.005-10% | 0.001-50% | 0.001-99% |
| Nutrient media | 10-50% | 3-70% | 1-85% | 0.3-99% |
| Organic liquid | 0.001-2% | 0.0003-1% | 0.0001-10% | 0.00003-99% |

### Formation of Particles

The particles as described herein, can be formed by contacting the liquid droplets that include a therapeutic biologic and a solvent with a third liquid that facilitates removal of the solvent from the liquid droplets. In some embodiments, the droplets are formed in a separate medium, e.g., the second liquid, and placed into contact with the third liquid thereafter. Particle formation begins to take place when at least a subset of the components of the liquid droplets begin to undergo precipitation or phase separation as the solvent is removed. In preferred embodiments, the droplets are dried after contacting the liquid droplets with a third liquid.

In some embodiments, particles are formed after the first liquid disperses throughout the third liquid, e.g., through a diffusion process. In other embodiments the third liquid may have varying degrees of miscibility with the solvent and represent a weakly or negligibly solubilizing medium in relation to the components of the particles or a subset of the components of the particles, e.g., the therapeutic biologic. The therapeutic biologic, e.g., antibody, bovine serum albumin (BSA), or human serum albumin (HSA), is typically less soluble in the third liquid relative to the first liquid in the timeframe of or under the conditions of production, e.g., at least about 5, 10, 100, or about 1000 times less soluble. In still other embodiments, the third liquid is an aqueous liquid, an organic solvent, an oil, an ionic liquid, or a combination thereof. In preferred embodiments, the third liquid is an organic solvent. The third liquid can further include a carbohydrate, a pH adjusting agent, a salt, a chelator, a mineral, a polymer, a surfactant, an amino acid, an oligopeptide, a biologic excipient, a chemical excipient, an antiseptic, an antioxidant, a paraben, a bactericide, a fungicide, a vitamin, a preservative, an analgesic, a nutrient media, or a combination thereof. Exemplary aqueous liquids may contain stabilizers, e.g., crowding agents. These solutions, in certain embodiments, include excipients such as a salt (e.g., sodium chloride), sugars and sugar alcohols (e.g., sorbitol, dextran 40, dextran 6000, or trehalose), polymers (e.g., PEG 3350, PEG 300, PEG 8000, PEG 20k, Ficoll 400, Ficoll 70, or polyvinylpyrrolidone, e.g., Povidone), a protein (e.g., bovine serum albumin (BSA), or human serum albumin (HSA)), or a combination thereof. In still other embodiments, where the first liquid, e.g., the solvent, is aqueous, particles are obtained via osmotic drying of the droplets. The third liquid that is used to dry the particles, in certain embodiments, include a high concentration of a solute (therapeutic biologic and/or excipient, e.g., surfactant), e.g., at least about 0.03 osmol, at least about 0.2 osmol, at least about 1.0 osmol, or at least about 1.2 osmol.

In other embodiments, the surfactant in the third liquid helps to prevent coalescence of the droplets. In certain embodiments, an oligopeptide excipient, a protein excipient, the therapeutic biologic(s) themselves, e.g., antibody, bovine serum albumin (BSA), or human serum albumin (HSA), or a combination thereof, act as surfactants.

In preferred embodiments, the liquid droplets of step c) are dried after contact with a third liquid.

The actual drying or desiccation time may similarly vary on account of changes that take place in the drop as concentration of the solutes, precipitation of solutes, and/or phase separation begin to take place. Depending on the chosen process conditions, drying of the particles may occur over a period of nanoseconds to days. In certain embodiments where the first liquid is aqueous and where the third liquid is an organic solvent, drying times vary, e.g., of about 1 μs and about 1000 s depending on the solvent chemistry. The term “polarity” or “polarities” refer to the overall solvation capability (solvation power) of the solvent, which in turn depends on the action of all possible, nonspecific and specific, intermolecular interactions between solute ions or molecules and solvent molecules, excluding, however, those interactions leading to definite chemical alterations of the ions of molecules of the solute (Chem. Rev., 1994, 94, 2319-2358). A prediction of solvent polarity may be made from their dielectric constant. Solvents with high dielectric constants are considered more polar and those with low dielectric constants are considered less polar or nonpolar (<˜15).

In some embodiments, the third liquid has a Fourier number (Fo) of less than about 1.500 allowing the liquid droplets to dry in about 60 seconds. In other embodiments, the third liquid has a Fourier number (Fo) of less than about 1.000 allowing the liquid droplets to dry in about 60 seconds. In still other embodiments, the third liquid has a Fourier number (Fo) of less than about 0.500 allowing the liquid droplets to dry in about 60 seconds. In certain other embodiments, the third liquid has a Fourier number (Fo) of less than about 0.208 allowing the liquid droplets to dry in about 5 seconds. The skilled person, once apprised of the range to be set for the Fourier number, will be able, without undue burden, to adjust the process parameters accordingly.

In other embodiments, the third liquid is an aqueous liquid, an organic solvent, an ionic liquid, a hydrogel, ionogel, protein stabilizer, or a combination thereof. In preferred embodiments, the third liquid is an organic solvent.

In some embodiments, the organic solvent is benzyl alcohol, benzyl benzoate, castor oil, coconut oil, corn oil, cottonseed oil, fish oil, grape seed oil, hazelnut oil, hydrogenated palm seed oil, olive oil, peanut oil, peppermint oil, safflower oil, sesame oil, soybean oil, sunflower oil, vegetable oil, walnut oil, polyethylene glycol, glycofurol, acetone, diglyme, dimethylacetamide, dimethyl isosorbide, dimethyl sulfoxide, ethanol, methyl acetate, ethyl acetate, propyl acetate, butyl acetate, ethyl ether, ethyl lactate, isopropyl acetate, methyl acetate, methyl isobutyl ketone, methyl tert-butyl ether, N-methyl pyrrolidone, perfluorodecalin, 2-pyrrolidone, trigylcerides, tetrahydrofurfuryl alcohol, triglycerides of the fractionated plant fatty acids C8 and C10 (e.g., MIGLYOL® 810 and MIGLOYL® 812N), propylene glycol diesters of saturated plant fatty acids C8 and C10 (e.g., MIGLYOL® 840), ethyl oleate, ethyl caprate, dibutyl adipate, fatty acid esters, hexanoic acid, octanoic acid, triacetin, diethyl glycol monoether, gamma-butyrolactone, eugenol, clove bud oil, citral, limonene, hexanes, heptane, or a combination thereof. In certain embodiments, the organic solvent is methylacetate, ethylacetate, propylacetate, butylacetate, amylacetate, 2-ethylhexylacetate, heptane, or a combination thereof. In preferred embodiments, the organic solvent is butylacetate.

In certain embodiments, the organic solvent is acetonitrile, chlorobenzene, chloroform, cyclohexane, cumene, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, methylisobutylketone, N-methylpyrrolidone, nitromethane, pyridine, sulfolane, tetrahydrofuran, tetralin, toluene, 1,1,2-trichloroethene, xylene, acetic acid, acetone, anisole, 1-butanol, 2-butanol, butylacetate, tert-butylmethyl ether, dimethyl sulfoxide, ethanol, ethylacetate, ethyl ether, ethyl formate, formic acid, heptane, isobutylacetate, isopropylacetate, methylacetate, 3-methyl-1-butanol, methylethyl ketone, 2-methyl-1-propanol, pentane, 1-pentanol, 1-propanol, 2-propanol, propylacetate, triethylamine, 1,1-diethoxypropane, 1,1-dimethoxymethane, 2,2-dimethoxypropane, isooctane, isopropyl ether, methylisopropyl ketone, methyltetrahydrofuran, petroleum ether, trichloroacetic acid, trifluoroacetic acid, decanol, 2-ethylhexylacetate, amylacetate, or a combination thereof.

In other embodiments, the third liquid further comprises a carbohydrate, a pH adjusting agent, a salt, a chelator, a mineral, a polymer, a protein stabilizer, an emulsifier, an antiseptic, an amino acid, an antioxidant, a protein, an organic solvent, a paraben, a bactericide, a fungicide, a vitamin, a preservative, a nutrient media, an oligopeptide, a biologic excipient, a chemical excipient, a surfactant, or a combination thereof. In preferred embodiments, the third liquid further comprises a surfactant.

In some embodiments, the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, phosphatidylcholine, polyglycerol polyricinoleate, siloxanes, cetyl polyethylene glycol/polypropylene glycol-10/1 dimethicone triglyceride, bis-polyethylene glycol/polypropylene glycol-14/14 dimethicone, bis-(glyceryl/lauryl) glyceryl lauryl dimethicone (&) caprylic/capric triglyceride, cetyl polyethylene glycol/polypropylene glycol-10/1 dimethicone, phospholipids, or a combination thereof. In certain embodiments, the surfactant is polysorbate, docusate or lecithin. In certain other embodiments, the surfactant is polysorbate 20, polysorbate 60, or polysorbate 80, e.g., Tween 20, Tween 60, Tween 80. In still other embodiments, the surfactant is polysorbate 20 or polysorbate 80. In certain preferred embodiments, the fatty acid ester of sorbitol is a sorbitan ester, e.g., span 20, span 40, span 60, or span 80. In other preferred embodiments, the surfactant is an ionic surfactant. In preferred embodiments, the surfactant is polysorbate 80.

As described herein, the use of surfactants can be a method for stabilizing liquid-liquid systems. In some embodiments, the droplets are stabilized on the timescale of and under the conditions of particle formation by addition of an appropriate surfactant to the first, second, and/or third liquid. In terms of the critical micelle concentration (CMC), the concentration of the surfactant is from about 0.01 to about 100 times the CMC, e.g., from about 0.1 to about 10 times the CMC, from about 1 to about 5 times the CMC, or about 1 to about 3 times the CMC. In other embodiments, the concentration of the surfactant is from about 0.0001 mg/mL to about 100 mg/mL, e.g., from about 0.001 mg/mL to about 10 mg/mL, from about 0.01 mg/mL to about 10 mg/mL, or from about 0.01 mg/mL to about 1 mg/mL. As used herein, the term “critical micelle concentration”, or “CMC”, refers to the concentration of surfactants in a liquid described above which micelles form and which all additional surfactants added to the system go to micelles.

In other embodiments, the third liquid has a viscosity of less than about 200 mPa·s, less than about 150 mPa·s, less than about 125 mPa·s, less than about 100 mPa·s, less than about 75 mPa·s, less than about 75 mPa·s, less than about 70 mPa·s, less than about 65 mPa·s, less than about 60 mPa·s, less than about 55 mPa·s, less than about 50 mPa·s, less than about 45 mPa·s, less than about 40 mPa·s, less than about 35 mPa·s, less than about 30 mPa·s, less than about 25 mPa·s, less than about 20 mPa·s, less than about 19 mPa·s, less than about 18 mPa·s, less than about 17 mPa·s, less than about 16 mPa·s, less than about 15 mPa·s, less than about 14 mPa·s, less than about 13 mPa·s, less than about 12 mPa·s, less than about 11 mPa·s, less than about 10 mPa·s, less than about 9.5 mPa·s, less than about 9 mPa·s, less than about 8.5 mPa·s, less than about 8 mPa·s, less than about 7.5 mPa·s, less than about 7 mPa·s, less than about 6.5 mPa·s, less than about 6 mPa·s, less than about 5.5 mPa·s, less than about 5 mPa·s, less than about 4.5 mPa·s, less than about 4 mPa·s, less than about 3.5 mPa·s, less than about 3 mPa·s, less than about 2.5 mPa·s, less than about 2 mPa·s, less than about 1.5 mPa·s, less than about 1 mPa·s, less than about 0.5 mPa·s, less than about 0.1 mPa·s, less than about 0.05 mPa·s, or less than about 0.01 mPa·s (one millipascal-second). In other embodiments, the third liquid has a viscosity of about 0.01 mPa·s to about 10,000 mPa·s, e.g., from about 0.01 mPa·s to about 1,000 mPa·s, from about 0.01 mPa·s to about 100 mPa·s, from about 0.01 mPa·s to about 50 mPa·s, from about 0.01 mPa·s to about 25 mPa·s, from about 0.01 mPa·s to about 10 mPa·s, from about 0.01 mPa·s to about 5 mPa·s, or from about 0.01 mPa·s to about 1 mPa·s. In certain embodiments, the third liquid has a viscosity that ranges from about 0.27 mPa·s to about 200 mPa·s, e.g., about 0.27 mPa·s to about 50 mPa·s, about 1 mPa·s to about 30 mPa·s, or about 20 mPa·s to about 50 mPa·s. In still other embodiments, the third liquid has a viscosity that ranges from about 0.27 mPa·s to about 200 mPa·s, e.g., about 0.27 mPa·s to about 100 mPa·s, about 0.27 mPa·s to about 50 mPa·s, about 0.27 mPa·s to about 30 mPa·s, about 1 mPa·s to about 20 mPa·s, or about 1 mPa·s to about 15 mPa·s. Methods of controlling viscosity include temperature regulation and viscosity modifying additives. Mixtures of liquids may also be used to control viscosity.

In some embodiments, the third liquid has a viscosity from about 0.01 mPa·s to about 10,000 mPa·s. In other embodiments, the third liquid has a viscosity of less than about 50 mPa·s. In still other embodiments, the third liquid has a viscosity of less than about 10 mPa·s. In certain other embodiments, the third liquid has a viscosity of less than about 5 mPa·s. In certain embodiments, the third liquid has a viscosity of less than about 2 mPa·s. In preferred embodiments, the third liquid has a viscosity of less than about 0.40 mPa·s.

In other embodiments as described herein, step c) further comprises decreasing the temperature of the third liquid to a temperature within about 30° C. of the freezing point of the first liquid. In some embodiments, the boiling point of the third liquid at atmospheric pressure is from about 0 to about 200° C. In certain embodiments the temperature, pressure, and vapor content of the medium, e.g., the second and third liquid, in which the droplets are dispersed can be regulated to control the evaporation characteristics. The temperature of the medium during evaporation is from about −100 to about 300° C., e.g., from about −100 to about 200° C., from about −100 to about 150° C., from about −100 to about 100° C., from about −75 to about 75° C., from about −40 to about 40° C., from about −30 to about 30° C., from about −20 to about 20° C., from about −10 to about 10° C., or from about −4 to about 4° C. The pressure of the medium during evaporation can be from about 10⁻⁶ atm to about 10 atm, e.g., from about 10⁻⁶ atm to about 1 atm, from about 10⁻⁵ atm to about 1 atm, from about 10⁻⁴ atm to about 1 atm, or from about 10⁻³ atm to about 1 atm. The vapor content of the medium during evaporation, relative to the saturation point, can be from about 0 to about 100%, e.g., from about 0 to about 50%, from about 0 to about 25%, from about 0 to about 10%, from about 0 to about 5%, from about 0 to about 2%, from about 0 to about 1%, from about 0 to about 0.5%, from about 0 to about 0.1%, or from about 0 to about 0.01%.

The term “primary desiccation” refers to a step by which a droplet comprising a first liquid is placed in contact with a third liquid and dried or desiccated by the third liquid, e.g., through dispersal of the first liquid in the third liquid, and/or through evaporation. The term “secondary desiccation” refers to a post-processing step, e.g., after removal of the first liquid, second liquid, and third liquid by which the residual moisture and/or first liquid, second liquid, and third liquid content of the particles is modified. Exemplary methods of secondary desiccation include vacuum drying, with or without the application of heat, lyophilization, fluidized bed drying, tray drying, belt drying, or slurry spray drying. Secondary desiccation may also be used to remove any washing liquids that are used to separate the particles from the first liquid, second liquid, and third liquid. In preferred embodiments, the first liquid, second liquid, and third liquid is removed through centrifugation, sieving, filtration, magnetic collection, solvent exchange, decanting, or a combination thereof. In certain preferred embodiments, the filtration is tangential flow filtration.

Droplets of the disclosure can be placed in contact with a third liquid in several ways. In preferred embodiments, the droplets are formed in the second liquid. In some embodiments, the contacting of the liquid droplets with a third liquid of step c) is accomplished by membrane emulsification, homogenization, mechanical stirring, mechanical shaking, impinging jet mixing, ultra-sound, sonication, micro-channel emulsification, microsieve emulsification, capillary extrusion, static mixing, or a combination thereof. In certain embodiments, the micro-channel emulsification is accomplished using a microfluidic chip based device. In other embodiments, the contacting of the liquid droplets with a third liquid of step c) is accomplished by membrane emulsification, homogenization, impinging jet mixing, static mixing, or a combination thereof. In certain embodiments, the membrane emulsification is conducted by rotating membrane emulsification, cross-flow membrane emulsification, or a combination thereof. In still other embodiments, the homogenization is conducted by shear homogenization, pressure homogenization, rotor-stator homogenization, microfluidization, or a combination thereof. A person of ordinary skill in the field of this disclosure can readily assess the shear homogenization or pressure homogenization of the disclosed methods using routine and standard techniques for high or how shear homogenization, or high or low pressure homogenization. In certain other embodiments, the mechanical stirring is conducted by a turbulent stirred vessel, a magnetic stirring device, a mechanical stirring device, or a combination thereof. In certain preferred embodiments, the static mixing comprises laminar flow, turbulent flow, transition flow, or a combination thereof.

In some embodiments, the third liquid includes or is in contact with a drying substance, i.e., a desiccant, to absorb the solvent or otherwise sequester it, e.g., by reaction. Such substances can be useful for ensuring a uniform, steady-state degree of saturation of the first liquid in the second liquid during drying. Exemplary desiccants include, but are not limited to celite, molecular sieves, phosphorous pentoxide, magnesium sulfate, silica, calcium chloride, activated charcoal, or potassium carbonate.

### Post-Processing

In some embodiments, the methods as described herein, include removing the particles from the first liquid, second liquid, and third liquid through centrifugation, sieving, filtration, magnetic collection, solvent exchange, inertial separation, hydrocyclone separation, or decanting. In other embodiments, the first liquid, second liquid, and third liquid is removed through centrifugation, sieving, filtration, magnetic collection, solvent exchange, decanting, or a combination thereof. In certain preferred embodiments, the filtration is tangential flow filtration.

In other embodiments, the methods as described herein, further comprises washing the particles after step e) with a washing fluid, e.g., an organic liquid, a supercritical fluid, a cryogenic liquid, or a combination thereof. In certain embodiments, the washing fluid is an organic liquid, a supercritical fluid, a cryogenic liquid, or a combination thereof.

The drying of the particles, e.g., after removing the first liquid, second liquid, and third liquid to produce dry particles, can be performed through methods as described herein. These include, but are not limited to, warm gas evaporation, freeze drying, critical point drying, emulsion solvent evaporation, emulsion solvent diffusion, or a combination thereof. In certain embodiments, the particles are further dried by lyophilization or vacuum desiccation. In certain other embodiments, residual quantities of the first liquid, second liquid, and third liquid in the particles after drying are from about 0 to about 10% by weight, e.g., from about 0 to about 5% by weight, or from about 0 to about 3% by weight, or preferably from about 0 to about 1% by weight. In still other embodiments, the particles have less than 10% residual quantities of the first liquid, second liquid, and third liquid by weight remaining after drying after removing the first liquid, second liquid, and third liquid.

In some embodiments, the particles are removed from the first liquid, second liquid, and third liquid through a solvent exchange washing procedure. After removal of most of the first liquid, second liquid, and third liquid (e.g., after centrifugation and supernatant decanting), a fourth liquid may be added which is volatile, miscible with the first liquid, second liquid, and third liquid, and in which the particles are not soluble under the conditions of washing. In other embodiments, the fourth liquid can be replaced with a more volatile washing liquid that is easier to remove. Additional cycles of concentration, supernatant removal, and backfilling with the washing liquid may lead to substantial reduction of the content of the first liquid, second liquid, and third liquid. The volatile washing liquid can be subsequently removed, e.g., by application of heat and/or vacuum, or removed via lyophilization. In certain embodiments, the volatile washing liquid is an organic liquid. In certain other embodiments, the volatile washing liquid is a supercritical fluid, e.g., supercritical CO₂, a cryogenic fluid, e.g., liquid nitrogen, or a mixture of one of these liquids and an organic liquid. In still other embodiments, the boiling point of the volatile washing liquid at atmospheric pressure is from about −200 to about 200° C., e.g., from about −200 to about 100° C., from about −200 to about 75° C., or from about −200 to about 50° C. In certain other embodiments, the methods described herein, further include washing the particles with a fourth liquid. In certain preferred embodiments, the fourth liquid is an organic solvent. The fourth liquid can also be removed through evaporation, vacuum desiccation or lyophilization, e.g., vacuum drying, with or without the application of heat, lyophilization, fluidized bed drying, tray drying, belt drying, or slurry spray drying. In preferred embodiments, the particles are further dried by lyophilization or vacuum desiccation.

In other embodiments, warm gas evaporation is used to further dry the particles. In some embodiments, the particles are further dried by contacting the particles with a stream of gas. In certain embodiments, the gas has a temperature from about −80 to about 200° C. In certain other embodiments, the gas has a temperature from about 10 to about 40° C. In still other embodiments, the gas has a relative humidity greater than about 0% to less than about 100%. In preferred embodiments, the gas comprises helium, air, nitrogen or argon.

The particles can be subjected to one or more secondary desiccation steps after separation from the first liquid, second liquid, and third liquid. Such steps can be utilized to remove volatile washing liquid, and/or to modulate residual quantities of the first liquid, second liquid, and third liquid in the particles. In certain embodiments, residual quantities of the first liquid, second liquid, and third liquid persist in the particles after primary desiccation. In certain other embodiments, secondary drying is useful for reducing quantities of the first liquid, second liquid, and third liquid to a desired level. Exemplary methods of secondary desiccation include vacuum drying with or without application of heat, lyophilization, fluidized bed drying, slurry spray drying, tray drying, belt drying, or air drying on a filter membrane.

In some embodiments, secondary desiccation is achieved by flowing a drying gas over a bed of particles atop a filtration element. In certain embodiments, the drying gas is helium, air, nitrogen or argon. In preferred embodiments, the drying gas is helium or air. The temperature, pressure, flow rate, or vapor content of the drying gas may be controlled during the drying time to achieve a desired rate of desiccation, a desired temperature difference relative to the glass transition temperature, or a desired equilibrium content of the first liquid or the second liquid at the conclusion of the secondary desiccation step. In other embodiments, the time required to achieve a desired level of desiccation is lower than that which corresponds to alternative secondary desiccation techniques, e.g., lyophilization, spray drying, or fluidized bed drying.

In other embodiments, the primary desiccation step, the washing step, and/or the secondary desiccation step are facilitated by modulating the temperature of particles relative to their glass transition temperature. Under certain conditions, quantities of the first liquid, the second liquid, the washing liquid, and/or various components of the drop or particle, e.g., a surfactant, become trapped in a “glassy” matrix during particle formation (Richardson, H. et al., The European Physical Journal E, 12, no. 1 (2003): 87-91). In some embodiments, removal of various trapped liquids and particle components can be facilitated by bringing the temperature of the particle in proximity to the glass transition temperature for a period of time. Proximity to the glass transition enhances mobility within the particle and permits liberation of the trapped liquids and particle components at a substantially enhanced rate relative to what is typically seen at temperatures well below that of the transition. With respect to the glass transition temperature, the temperature of the particles during step d) can be within about ±30° C., within about ±20° C., within about ±10° C., within about ±5° C., within about ±2° C., or within about ±1° C. The duration for which the particle must be held in this proximity can vary as a function of the mobility of the liquid and particle component to be extracted and the conditions of extraction, e.g., the temperature, flow rate, and humidity of the drying gas, but can be from about 0 to about 24 hours, from about 0 to about 12 hours, from about 0 to about 6 hours, from about 0 to about 3 hours, from about 0 to about 1 hour, from about 0 to about 0.5 hours, from about 0 to about 0.25 hours, or from about 0 to about 0.1 hours.

In some embodiments, the particles have less than about 5% of residual moisture by mass. In other embodiments, the particles have less than about 3% of residual moisture by mass. In certain other embodiments, the particles have less than about 2% of residual moisture by mass. In preferred embodiments, the particles have less than about 1% of residual moisture by mass. In certain preferred embodiments, the particles are substantially free from any residual moisture by mass. In some embodiments, the particles have less than about 0.1% of residual moisture by mass. In some embodiments, the particles have less than about 0.01% of residual moisture by mass. Exemplary methods for the measurement of moisture content include chemical titration methods, e.g., Karl Fischer titration involving an oven. A variety of solvents, including water, may also be measured using weight loss methods involving thermal excitation. Exemplary methods include Thermogravimetric Analysis with Infrared Spectroscopy (TGA-IR) or Gas Chromatography Flame Ionization Detector Mass Spectrometry (GC-FID/MS).

Sterility: Sterility is a critical facet of pharmaceutical compositions because it affects the safety with which the composition may be administered. For example, many particle formulations, particularly microparticle formulations, achieving sterility can be a challenge since common sterilization techniques, e.g., sterile filtration, are not compatible. Sterile filtration steps typically involve a membrane through which only those components of the filtered liquid which are, for example, 200 nm in size or smaller may pass. Particle formulations with solids greater than 200 nm in size are therefore filtered rather than sterilized. In some embodiments, formulations of the disclosure are subjected to an alternative process of terminal sterilization prior to use or administration. The effectiveness of these sterilization protocols and of the process in reducing bioburden may be assessed following regulatory guidelines, e.g., those listed in USP Chapter <71>, Ph. Eur. Chapter, Sterility: 2.6.1, 21 CFR 610.12, ICH Q4B ANNEX 8(R1), ICH Q5A, etc. Exemplary methods of demonstrating compliance include incubating about 1 mL of the drug product per container in an appropriate growth media (Soybean-Casein Digest Medium, Tryptic Soy Broth, Fluid Thioglycollate Medium) for a period of about 14 days to ensure no microbial growth in about 1 in about 1000 million units of the drug product, or about 1 in about 1 million units of the drug product. As disclosed herein, a “sterile” formulation is aseptic or free from living microorganisms and their spores. In preferred embodiments, the methods described herein, further comprises sterilization of the particles after the first liquid, second liquid, and third liquid is removed. In certain embodiments, the sterilization occurs by irradiation, pasteurization, or freezing. In certain preferred embodiments, the irradiation is by gamma radiation.

In some embodiments, the terminal sterilization step involves gamma irradiation. In other embodiments, the sterilization step required to inactivate at least about 2-4 log₁₀ of viral microbial contaminants is about 10 kGy, about 20 kGy, about 40 kGy, about 60 kGy, or about 100 kGy. In certain embodiments, the particles comprise an antioxidant or a scavenger to mitigate the harmful effects of any degradation products which are generated as a result of the sterilization step.

In other embodiments, the terminal sterilization step involves a transient thermal treatment. In some embodiments, the formulation is exposed to temperatures from about 60 to about 200° C., e.g., from about 60 to about 180° C., from about 60 to about 160° C., from about 60 to about 140° C., from about 60 to about 130° C., from about 60 to about 120° C., or from about 60 to about 110° C. In certain embodiments, the exposure occurs over a period from about 1 to about 144 hours, e.g., from about 1 to about 120 hours, from about 1 to about 100 hours, from about 1 to about 90 hours, from about 1 to about 72 hours, from about 1 to about 48 hours, from about 1 to about 36 hours, or from about 1 to about 24 hours. For example, dry heat sterilization can be performed at a temperature of about 80° C. for about 72 hours, about 160° C. for about two hours, or about 170° C. for about one hour. In certain other embodiments, pasteurization is performed at about 60° C. for about 10 hours.

In some embodiments, the sterilization is ensured by using beta radiation, X-ray sterilization, steam sterilization, solvent-detergent inactivation steps, supercritical CO₂ mediated sterilization, low pH holds, ultraviolet C exposure, or ethylene oxide mediated sterilization of the formulation. In other embodiments, the terminal sterilization step is performed at low temperatures from about −100 to about 60° C. In certain embodiments, the supercritical CO₂ further includes additives (e.g., hydrogen peroxide, water, acetic anhydride, etc.) intended to effectively inactivate microorganisms, including bacterial spores.

In other embodiments, the third liquid is chosen such that its presence helps to facilitate process sterility. In some embodiments, the third liquid is an antimicrobial or contains such a compound which is contained within the particle. This compound may persist inside the particles even after the secondary drying step. Organic liquids that can be used as a third liquid with antimicrobial activity may include, but are not limited to acetates (e.g., ethyl acetate, butyl acetate) and alcohols (e.g., ethanol, phenol), or the like. The third liquid may also contain antimicrobial excipients, e.g., phenolic substances, benzalkonium chloride, linalool, coumarin, peroxides, active chlorine, alkalis, or a combination thereof.

In some embodiments, the use of nano-filtration membranes for the inlet process streams, e.g., for use on the first liquid, second liquid, and/or third liquid prior to particle formation, contributes to a reduction of the bio-burden on the process. In other embodiments, combinations of the preceding sterility measures are employed to reach appropriate bio-burden levels.

As described herein, the particles may be sterilized after formation, e.g., by irradiation, pasteurization, freezing, or irradiation by gamma radiation. In certain embodiments, the methods as described herein, further comprises sterilization of the particles after the first liquid, second liquid, and third liquid is removed. In certain preferred embodiments, the sterilization occurs by irradiation, pasteurization, or freezing. In preferred embodiments, the irradiation is by gamma radiation.

### Control of Particle Properties

Properties of the particles can be controlled by modulating the drying rate of the droplets, the Peclet numbers of the components of the droplets, the concentrations of the components of the droplets, the particle formation dynamics following solute precipitation and/or phase separation within the droplet. In certain embodiments, the modulation influences the size, morphology, density, porosity, composition, surface energy properties of the particles, and help to establish the distribution of components within the particles and to regulate important physicochemical properties which may be difficult to address when drying without the third liquid, e.g., in air, as with conventional spray drying.

The methods described herein, are generally provided for controlling the morphology of particles, the method comprising: a) providing a first liquid comprising a therapeutic biologic and a solvent; b) contacting the first liquid with a second liquid, thereby forming liquid droplets comprising the therapeutic biologic; c) contacting the liquid droplets with a third liquid, thereby forming a mixture, wherein the Peclet number of the mixture determines the morphology of the particles; d) allowing the liquid droplets to dry; and e) removing the first liquid, second liquid, and third liquid, thereby forming the particles comprising a therapeutic biologic, wherein the particles comprise less than about 10% internal void spaces and the circularity of the particles is from about 0.80 to about 1.00 after removing the first liquid, second liquid, and third liquid.

In one aspect, the disclosure provides a method of controlling the morphology of particles, the method comprising: a) providing a first liquid comprising a therapeutic biologic and a solvent; b) contacting the first liquid with a second liquid, thereby forming liquid droplets comprising the therapeutic biologic; c) contacting the liquid droplets with a third liquid, thereby forming a mixture, wherein the Peclet number of the mixture determines the morphology of the particles; d) allowing the liquid droplets to dry; and e) removing the first liquid, second liquid, and third liquid, thereby forming particles comprising a therapeutic biologic, wherein the particles comprise less than about 10% internal void spaces and the circularity of the particles is from about 0.80 to about 1.00 after removing the first liquid, second liquid, and third liquid. As disclosed herein, the therapeutic biologic may be an antibody. In certain embodiments, the therapeutic biologic has an activity per unit of about 0.5 to about 1.0. In certain preferred embodiments, the therapeutic biologic is a therapeutic biologic. In preferred embodiments, the therapeutic biologic in the particles has an activity per unit of about 0.8 to about 1.0.

In certain embodiments, the particle includes less than 10% internal void spaces, less than 5% internal void spaces, less than 1% internal void spaces, less than 0.1% internal void spaces, or less than 0.01% internal void spaces after removing the first liquid, second liquid, and third liquid. In preferred embodiments, the particle is substantially free from any internal void spaces after removing the first liquid, second liquid, and third liquid.

In other embodiments, the circularity of the particles is from about 0.85 to about 1.00 after removing the first liquid, second liquid, and third liquid. In still other embodiments, the circularity of the particles is from about 0.90 to about 1.00 after removing the first liquid, second liquid, and third liquid. In certain other embodiments, the circularity of the particles is from about 0.95 to about 1.00 after removing the first liquid, second liquid, and third liquid. In preferred embodiments, the circularity of the particles is from about 0.98 to about 1.00 after removing the first liquid, second liquid, and third liquid.

In some embodiments, the first liquid, second liquid, and third liquid further comprises a plasticizer that controls the morphology of the particles. Exemplary plasticizers include sucrose, xylitol, sorbitol, fructose, triglyceride, pectin, glycerol, triethylcitrate, ethyl acetate, citric acid, oleic acid, hydroxypropyl cellulose, methyl pyrrolidone polyethylene glycol, polypropylene glycol, polysorbate 80, diethyl phthalate and other phthalate derivatives, castor oil, triacetin, water, chlorpheniramine, 1-butyl-3-methyl imidazolium dioctyl sulfosuccinate, hexyl acetate, water, 2-ethylhexyl acetate, triethyl citrate, dibutyl sebacate, benzyl alcohol, benzyl benzoate, dimethylacetamide, various aqueous liquids, organic liquids, oils, ionic liquids, polysaccharides, sugars, diols, polyols, fatty acids, fatty acid esters, esters, surfactants, or a combination thereof. In certain embodiments, the plasticizer is sucrose, xylitol, sorbitol, fructose, triglyceride, pectin, glycerol, triethylcitrate, ethyl acetate, citric acid, oleic acid, hydroxypropyl cellulose, methyl pyrrolidone polyethylene glycol, polypropylene glycol, polysorbate 20, polysorbate 60, polysorbate 80, fatty acid ester of sorbitol, diethyl phthalate and other phthalate derivatives, castor oil, triacetin, water, chlorpheniramine, 1-butyl-3-methyl imidazolium dioctyl sulfosuccinate, hexyl acetate, 2-ethylhexyl acetate, triethyl citrate, dibutyl sebacate, benzyl alcohol, benzyl benzoate, dimethylacetamide, or a combination thereof. In preferred embodiments, the plasticizer is polysorbate 20, polysorbate 60, or polysorbate 80. In certain preferred embodiments, the plasticizer is polysorbate 20 or polysorbate 80. In certain other embodiments, the fatty acid ester of sorbitol is a sorbitan ester, e.g., span 20, span 40, span 60, or span 80.

Morphology: The morphology of the particle can be an important factor for certain applications in particle formation, as described herein. In order to minimize the viscosity of the suspension at a given particle concentration, it can be advantageous to minimize the degree to which the particles comprise internal void spaces (e.g., porosity) or exhibit irregular shapes.

As described herein, the Peclet number is regulated to control the particle morphology. The term “Peclet number” refers to the ratio of the rate of a solvent mass transport process outside of a droplet or particle to the rate of a solute mass transport process inside a droplet or particle. Exemplary Peclet numbers as described herein, during the drying period are about 1 or less, indicating a regime where transport of solutes within the drop is fast as compared to the radial velocity of the receding droplet surfaces. Such Peclet numbers tend to correlate with regular, circular particle morphologies. For Peclet numbers of about 1 or greater, the droplet surface tends to move fast in relation to the solutes, thereby leading to an enriched layer of solute near the surface of the drop. Situations of this type typically correlate with irregular particle morphologies, i.e., morphologies which are less smooth, less circular, and/or more porous than those associated with lower Peclet numbers. Such morphologies may comprise raisin-like features (high roughness) and/or increased internal void space. The Peclet number can be regulated in several different ways using various properties of the first liquid, second liquid, and third liquid. Such properties include the solubility of the first liquid in the third liquid as well as the initial saturation level of the third liquid. Furthermore, the diffusivity of the first liquid and third liquid may be controlled. Parameters which influence these properties include the temperature, viscosity, and/or polarity of the first liquid and/or the third liquid, as well as the surface tension at the interface between the first liquid and the third liquid. In certain embodiments, the third liquid is a mixture of two or more liquids of different polarities. In certain preferred embodiments, the mixture comprises liquids wherein the mixture comprises liquids having differing solubility.

In some embodiments, the Peclet number of the mixture of step c) is less than about 500. In other embodiments, the Peclet number of the mixture of step c) is less than about 10. In certain embodiments, the Peclet number of the mixture of step c) is less than about 5. In still other embodiments, the Peclet number of the mixture of step c) is less than about 3. In certain other embodiments, the Peclet number of the mixture of step c) is less than about 2. In preferred embodiments, the Peclet number of the mixture of step c) is less than about 1. The Peclet number can be calculated by methods known to one skilled in the art.

In certain embodiments, the temperature of the first liquid, second liquid, and/or third liquid can be controlled. The first liquid, second liquid, and third liquid may be kept at the same temperature or at different temperatures. In other embodiments, the temperature of each liquid is, independently, from about −100 to about 300° C., e.g., from about −20 to about 180° C., from about 0 to about 100° C., from about 0 to about 50° C., from about 0 to about 40° C., from about 0 to about 30° C., from about 0 to about 20° C., from about 0 to about 10° C., or from about 0 to about 5° C. In preferred embodiments, the temperature of each liquid is from about 0 to about 20° C., from about 0 to about 10° C., or from about 0 to about 5° C.

In other embodiments, the viscosity of the first liquid, second liquid, and third liquid can be controlled. In some embodiments, the viscosity of the first liquid, second liquid, and/or third liquid affects a coefficient of diffusion or dispersal of the first liquid in the third liquid, thereby regulating the drying rate and Peclet number. The viscosity of each liquid may be, independently, from about 0.01 mPa·s to about 10,000 mPa·s, e.g., from about 0.01 to about 1,000 mPa·s, from about 0.01 to about 100 mPa·s, from about 0.01 to about 50 mPa·s, from about 0.01 to about 25 mPa·s, from about 0.01 to about 10 mPa·s, from about 0.01 to about 5 mPa·s, or from about 0.01 to about 1 mPa·s. Methods of controlling viscosity may include temperature regulation and viscosity modifying additives, e.g., polymers. Mixtures of liquids may also be used to control viscosity.

In some embodiments, the solvent polarity of the first liquid, second liquid, and third liquid is controlled. In other embodiments, the first liquid has a dielectric constant from about 1 to about 200, e.g., from about 1 to about 180, from about 10 to about 140, from about 30 to about 120, from about 50 to about 100, or from about 70 to about 80. In still other embodiments, the third liquid has a dielectric constant from about 1 to about 200, e.g., from about 1 to about 10, from about 1 to about 80, from about 1 to about 50, from about 1 to about 40, from about 1 to about 30, from about 1 to about 20, from about 1 to about 10, from about 1 to about 7, from about 1 to about 5, or from about 1 to about 3. Mixtures of various liquids may be used to control polarity.

In other embodiments, the surface tension at the interface between the first liquid, second liquid, and third liquid is controlled. In some embodiments, the surface tension is from about 0 to about 100 mN/m, e.g., from about 0 to about 70 mN/m, from about 0 to about 60 mN/m, from about 0 to about 50 mN/m, from about 0 to about 40 mN/m, from about 0 to about 30 mN/m, from about 0 to about 20 mN/m, from about 0 to about 10 mN/m, from about 0 to about 9 mN/m, from about 0 to about 8 mN/m, from about 0 to about 7 mN/m, from about 0 to about 6 mN/m, from about 0 to about 5 mN/m, from about 0 to about 4 mN/m, from about 0 to about 3 mN/m, from about 0 to about 2 mN/m, or from about 0 to about 1 mN/m.

In some embodiments, the third liquid is a mixture of two or more liquids. In other embodiments, the mixture is used to tune the viscosity and/or polarity of the third liquid. In certain embodiments, the mixture is used to tune the solubility of the first liquid in the third liquid. Since such properties can affect the rate and Peclet number associated with the drying process, they may be used to directly control various particle properties (e.g., size, morphology, skeletal density, etc.) through simple adjustment of the relative ratios of the liquids comprising the mixture. For example, a mixture for which the first liquid is more soluble in one component (Component A) than the other (Component B). In certain other embodiments, increasing the relative quantify of Component B can yield particles which are smoother, more spherical, and/or less porous than what would otherwise be achievable using only Component A. In still other embodiments, for mixtures, one liquid in the mixture has a concentration from about 0 to about 99.9999 vol %, e.g., from about 0 to about 99 vol %, from about 0 to about 95 vol %, from about 0 to about 90 vol %, from about 0 to about 75 vol %, from about 0 to about 50 vol %, from about 0 to about 25 vol %, from about 0 to about 10 vol %, from about 0 to about 5 vol %, from about 0 to about 1 vol %, or from about 0 to about 0.0001 vol %. Exemplary mixtures include, but are not limited to benzyl benzoate/acetone (e.g., about 5-30% benzyl benzoate, such as about 5:95, about 10:90, about 15:85, about 20:80, about 25:75, or about 30:70), isopropyl alcohol/sesame oil (e.g., about 35-65% isopropyl alcohol, such as about 35:65, about 40:60, about 45:55, about 50:50, about 55:45, about 60:40, or about 65:35), hexanes/ethanol (e.g., about 10-35% hexanes, such as about 10:90, about 15:85, about 20:80, about 25:75, about 30:70, or about 35:65), toluene/acetonitrile (e.g., about 10-35% toluene, such as about 10:90, about 15:85, about 20:80, about 25:75, about 30:70, or about 35:65), cottonseed oil/butyl acetate (e.g., about 10-35% cottonseed oil, such as about 10:90, about 15:85, about 20:80, about 25:75, about 30:70, or about 35:65), toluene/ethyl acetate (e.g., about 10-35% toluene, such as about 10:90, about 15:85, about 20:80, about 25:75, about 30:70, or about 35:65), diethyl ether/isopropanol (e.g., about 5-30% diethyl ether, such as about 5:95, about 10:90, about 15:85, about 20:80, about 25:75, or about 30:70), tetrahydrofuran/pentane (e.g., about 35-65% THF, such as about 35:65, about 40:60, about 45:55, about 50:50, about 55:45, about 60:40, or about 65:35), safflower oil/methanol (e.g., about 25-55% safflower oil, such as about 25:75, about 30:70, about 35:65, about 40:60, about 45:55, about 50:50, or about 55:45), and lime oil/acetone (about 5-30% lime oil, such as about 5:95, about 10:90, about 15:85, about 20:80, about 25:75, or about 30:70). As described herein, choosing the appropriate liquid combinations and ratios, e.g., components of the third liquid, can control the particle drying speed and Peclet number.

In other embodiments, the mixture comprising the third liquid further includes a surfactant. In some embodiments, the surfactant helps to establish an interface between the first and third liquid, and in other embodiments, to regulate the drying speed and Peclet number. In certain embodiments, the surfactant limits coalescence of the drops during the drying process and/or mitigates damage to the therapeutic biologic, e.g., an antibody, bovine serum albumin (BSA), or human serum albumin (HSA), at the interface between the first liquid and the third liquid. The concentration of the surfactant in the third liquid ranges from about 0 to about 100 vol %, e.g., from about 0 to about 50 vol %, from about 0 to about 25 vol %, from about 0 to about 10 vol %, from about 0 to about 5 vol %, from about 0 to about 1 vol %, from about 0 to about 0.1 vol %, from about 0 to about 0.01 vol %, from about 0 to about 0.001 vol %, or from about 0 to about 0.0001 vol %. Exemplary mixtures of third liquid and surfactant include, but are not limited to Polysorbate 80/ethyl acetate (e.g., about 0.5:95.5, such as about 0.1:99.9, about 1:99, about 2.5:97.5, about 5:95, about 10:90, about 20:80), Span 20/ethyl acetate (about 0.5:95.5, such as about 0.1:99.9, about 1:99, about 2.5:97.5, about 5:95, about 10:90, about 20:80), Polysorbate 20/ethyl acetate (e.g., about 0.5:95.5, such as about 0.1:99.9, about 1:99, about 2.5:97.5, about 5:95, about 10:90, about 20:80), Polysorbate 80/butyl acetate (e.g., about 0.5:95.5, such as about 0.1:99.9, about 1:99, about 2.5:97.5, about 5:95, about 10:90, about 20:80), Polysorbate 80/isopropanol (e.g., about 0.5:95.5, such as about 0.1:99.9, about 1:99, about 2.5:97.5, about 5:95, about 10:90, about 20:80), or Polysorbate 80/cottonseed oil/ethyl acetate (e.g., about 0.5:20:79.5, such as about 0.1:20:79.9, about 1:30:69, about 2.5:10:87.5, about 5:5:90, about 10:5:75, about 20:20:60).

Effective plasticization requires the use of a plasticizer in the first liquid, second liquid, and/or third liquid at a temperature at about or higher than the glass transition temperature of the plasticizer. Controlling the temperature of the plasticizer during the particle formation process can be achieved by controlling the temperature of the first liquid, second liquid, and/or third liquid. Effective plasticization can be achieved to obtain a smoother, more circular, and less porous particle morphology in instances where a component of the first liquid, e.g., a therapeutic biologic, is typified by a Peclet number greater than 1, and where the absence of effective plasticization would otherwise lead to particle morphologies which are more characteristic of high Peclet number processes.

In other embodiments, various aqueous liquids, organic liquids, oils, ionic liquids, polysaccharides, sugars, diols, polyols, fatty acids, fatty acid esters, esters, surfactants, or a combination thereof, are employed as effective plasticizers under appropriate processing conditions. Exemplary plasticizers include, but are not limited to sucrose, xylitol, sorbitol, fructose, triglyceride, pectin, glycerol, triethylcitrate, ethyl acetate, citric acid, oleic acid, hydroxypropyl cellulose, methyl pyrrolidone polyethylene glycol, polypropylene glycol, polysorbate 80, diethyl phthalate or other phthalate derivatives, castor oil, triacetin, water, chlorpheniramine, 1-butyl-3-methyl imidazolium dioctyl sulfosuccinate, hexyl acetate, water, 2-ethylhexyl acetate, triethyl citrate, dibutyl sebacate, benzyl alcohol, benzyl benzoate, dimethylacetamide, or a combination thereof.

Like plasticization, increasing the total solute load of the first liquid can be useful for achieving smoother, more spherical, and less porous particles in instances where the dynamics of particle formation are such that surface roughness may be likely to prevail at the nominal solute concentration. Similarly, decreasing the solute load of the first liquid can be leveraged to induce or encourage buckling when it might not otherwise prevail at the nominal solute load.

Control of the methods described herein, may be useful for achieving low Peclet numbers even in instances where a component or components of the first liquid, e.g., the therapeutic biologic, are characterized by having low diffusivity. In some embodiments, low Peclet numbers is achieved when the diffusivity of the component is from about 0 to about 10,000 μm²/s, e.g., from about 0 to about 1,000 μm²/s, from about 0 to about 100 μm²/s, from about 0 to about 50 μm²/s, from about 0 to about 25 μm²/s, from about 0 to about 10 μm²/s, from about 0 to about 5 μm²/s, from about 0 to about 2.5 μm²/s, or from about 0 to about 1 μm²/s.

The disclosure generically described, will be more readily understood by reference to the following examples, which are included merely for purposes of illustration of certain aspects and embodiments of the present disclosure, and are not intended to be limiting.

### EXEMPLIFICATION

### Abbreviations

Å angstrom
aa amino acids
BA butylacetate
agg aggregation
AU arbitrary units
BIgG bovine IgG
BSA bovine serum albumin
° C. degrees Celsius
cm centimeter
conc. concentration
cP centipoise
CP continuous phase
d day
DCM dichloromethane
DI deionized
DIPEA diisopropylethylamine
DMA N,N-dimethylaniline
DMF dimethyl formamide
DMSO dimethyl sulfoxide
DP dispersed phase
DTE dithioerythritol
DTT dithiothreitol
EDT 1,2-ethanedithiol
EDTA ethylenediaminetetraacetic acid
EO ethyloleate
eq. equivalent
Et ethyl
eV electron-volts
g gram
h hour
HIgG human IgG
HPLC high performance liquid chromatography
hr hour
HSA human serum albumin
Hz hertz
ID internal diameter
IV intravenous
KF Karl Fischer
kJ kilojoules
kPa kiloPascal
kV kilovolts
LC-MS liquid chromatograph mass spectrometry
L liter
m meta
mAb monoclonal antibody
MALDI-MS matrix-assisted laser desorption ionization mass spectrometry
Me methyl
MHz megahertz
min minute
microgram
microliter
μm micrometer
μM micromolar
mg milligram
mL milliliter
mm millimeter
mM millimolar
mol mole
mPa·s milliPascal second
mTorr milliTorr
N newton
nBA n-butylacetate
nm nanometer
NMP N-methylpyrrolidone
p para
PBS phosphate-buffered saline
PEG polyethylene glycol
PEGA polyethylene glycol polyacrylamide
PI pressure indicator
ppm parts per million
ps picosecond
PTFE polytetrafluoroethylene
rcf relative centrifugal force
RH relative humidity
RP-HPLC reversed phase-high performance liquid chromatography
rpm revolutions per minute
RT room temperature
s second
SC subcutaneous
sec second
SEM scanning electron microscopy
SVP subvisible particle
t tertiary
tert tertiary
UHMW ultrahigh molecular weight polyethylene
ug micrometer
UTW ultra thin wall
UOM unit of measure
UV ultraviolet
V volts
vol % volume percent
v/v volume per volume
W watt
wt % weight percent
w/v weight per volume
w/w weight per weight
wt/wt weight per weight

### Materials

Human IgG (IRHUGGF-LY, >97%) and bovine IgG (IRBVGGF) were obtained from Innovative Research as a powder. Bovine serum albumin (BSA) and human serum albumin (HSA) were purchased from Sigma-Aldrich. The monoclonal antibodies (mAb) were provided and received as an aqueous solution. Composition of custom “feed solutions” used for processing particles were produced through modifying the received formulation by desalting followed by concentrating and adding desired excipients or by direct buffer exchange. All excipients used in particle composition have been used in existing approved biologics injections. Concentration columns were procured from Millipore Sigma (Amicon® Ultra 15 mL Filters for Protein Purification and Concentration with a 3 kDa cut off) and used where necessary to: (i) reach the desired monoclonal antibody concentration, and (ii) exchange buffer/excipients before particle formation. Zeba desalting columns (Thermo Fisher Scientific 87773) were also used to remove salt from solutions in certain instances. Typically, the ratio of residual salt to therapeutic biologic in the desalted solutions (wt/wt) was <1%. All excipients were purchased from Sigma-Aldrich and used as received.

Desiccation liquids, i.e., third liquids, including benzyl benzoate, various alcohols, various acetates, oils, ionic liquids, surfactants, and aqueous media comprising different forms of polyethylene glycol (PEG) were used as appropriate. All desiccation (“third”) liquids, e.g., acetonitrile, chlorobenzene, chloroform, cyclohexane, cumene, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, methylisobutylketone, N-methylpyrrolidone, nitromethane, pyridine, sulfolane, tetrahydrofuran, tetralin, toluene, 1,1,2-trichloroethene, xylene, acetic acid, acetone, anisole, 1-butanol, 2-butanol, butylacetate, tert-butylmethyl ether, dimethyl sulfoxide, ethanol, ethylacetate, ethyl ether, ethyl formate, formic acid, heptane, isobutylacetate, isopropylacetate, methylacetate, 3-methyl-1-butanol, methylethyl ketone, 2-methyl-1-propanol, pentane, 1-pentanol, 1-propanol, 2-propanol, propylacetate, triethylamine, 1,1-diethoxypropane, 1,1-dimethoxymethane, 2,2-dimethoxypropane, isooctane, isopropyl ether, methylisopropyl ketone, methyltetrahydrofuran, petroleum ether, trichloroacetic acid, trifluoroacetic acid, decanol, 2-ethylhexylacetate, amylacetate, except for the ionic liquids were purchased from Sigma Aldrich and used as received. The ionic liquids were purchased from TCI America and used as received.

### Methods

Particle Formation: Unless otherwise noted, membrane emulsification, homogenization, mechanical stirring, mechanical shaking, impinging jet mixing, ultra-sound, sonication, micro-channel emulsification, microsieve emulsification, capillary extrusion, static mixing, or a combination thereof, was used for droplet formation and drying. A Harvard Apparatus Model 33 dual-channel syringe pump was utilized for pumping the feed solution (DP). The droplets generated in the second liquid were collected for desiccation by a vessel containing the third liquid, typically under conditions of continuous stirring. Thermal management of the third liquid was utilized in the preparation of select examples.

Lyophilization: The particles for lyophilized samples, i.e., samples marked as having gone through a secondary desiccation step involving freeze drying, were loaded into either microcentrifuge or 15 mL conical tubes and subjected to snap freezing by immersion in liquid nitrogen for approximately 10 min. The samples were then loosely covered and transferred to either a Virtis Advantage or a Labconco FreeZone lyophilizer for approximately 24 hours at a pressure of approximately 10-50 mTorr.

FlowCam: Particle sizing was measured using FlowCam; a dynamic image analysis instrument. Samples were typically diluted to about 1 mg/mL in isopropanol and passed through a thin channel. Images of particles were recorded and analyzed according to size and shape.

ImageJ Measurements: Particle diameters and circularity were measured using ImageJ analysis on SEM images. The analysis was performed on 600× images. The ImageJ Particle Analysis tool was run on the image, identifying objects with a circularity of >0.8 and size >0.5 μm with each object outlines. These outlines were visually inspected for good fit. Any mis-identified particles were manually rejected and any missed particles were manually included and measured using the ImageJ diameter tool.

Accelerated Storage Protocol: All samples were transferred to Wheaton E-Z extraction round-bottom glass vials for aging (typically 2 mL or 4 mL volume, depending on sample). The glass vials were sealed with parafilm, placed in an oven at 40° C., and visually inspected on a daily basis over the aging period to ensure integrity and stability.

Viscosity Measurements: Apparent suspension viscosity was measured using an AR-G2 rheometer (TA Instruments) and a 25 mm plate at 25° C. Measurements were taken at 1000 s-1 (experimental limit due to edge effects), which is below the shear rates experienced in 27-gauge needles. Each measurement was repeated three times (about 60 s intervals between repeats) to assess short-term physical stability of the suspensions. Prior to each measurement calibration standards were recorded to validate instrument settings.

Karl Fischer: Testing for moisture content was undertaken using Karl Fischer analysis. Approximately 10 mg of particles was heated to 165° C. in an oven and released water was determined coulometrically.

Skeletal Density: Skeletal density was measured by gas pycnometry. The gas was nitrogen or helium or other compatible gases, and the particle mass was about 0.0413 g to about 0.06 g, at about 22° C.

Particle Dissolution: Phosphate-buffered saline (PBS) was added to dry particle samples to produce a final concentration of 10 mg/mL (particle mass/mL of solution). Samples were placed on a VWR angular rocker with a speed setting of “35” and angle setting of “15”. At 1, 10, 20, 30, 40, 50, 60, 90, and 120 minutes a 10 μL aliquot was removed from the sample vial and the absorbance at 280 nm was measured and recorded. The mAb concentration was plotted against time for all samples.

Salt Content: Salt content was recorded by measuring sodium content using Inductively Coupled Plasma Optical Emission Spectroscopy (ICP-OES). A calibration curve was prepared using a sodium standard (ICPTraceCERT, 1000 mg/L). Quality control was completed using a diluted standard solution at 100 ppm sodium. A sample of particles (˜15 mg) dissolved in 2 vol % nitric acid (10 mL) was then analyzed, resulting in an intensity lower than the instrument detection limit of ˜0.5 ppm for sodium. This indicated a sodium content of less than 0.034 wt % and a total salt content (assuming sodium citrate and sodium chloride to have been removed equally) of less than 0.1 wt %.

Size Exclusion Chromatography (SEC) Measurements: The quantification of size variants in select samples was determined by size exclusion chromatography. The analysis utilized an AdvanceBio SEC-3 column, 7.8 mm ID×30 cm, 3 μm (Agilent) run on an HPLC system (1260 Infinity II, Agilent). The mobile phases were 25 mM potassium phosphate and 0.25 M potassium chloride at pH 6.8. The chromatography was run isocractically at a flow rate of 1.0 mL/min for 15 minutes. The column temperature was maintained at ambient temperature and the eluent absorbance was monitored at 280 nm. Monoclonal antibodies were diluted with its respective formulation buffer to 1 mg/mL. The injection volume was 10 μL. 20 μL Injections of samples (1 mg/mL) were run at a flow rate of 1 mL/min in SEC buffer (25 mM phosphate, 250 mM NaCl pH 6.8) for 15 minutes on an Agilent AdvanceBio SEC (300 mm×2.7 μm, 300 Å column). Peak analysis was performed by auto-integrating using the following parameters: slope sensitivity=0.5, peak width=0, height reject=0, area reject=0, shoulders off, area percent reject 0, standard tangent skim mode, advanced baseline correction, 0 for front peak skim height ratio, 0 for tail peak skim height ratio, 0 for peak to valley ratio, and 0 for skim valley ratio. Alternatively, 20 μL Injections of samples (1 mg/mL) were run at a flow rate of 1 mL/min in SEC buffer (25 mM phosphate, 250 mM NaCl pH 6.8) for 15 minutes on an Agilent AdvanceBio SEC (300 mm×2.7 μm, 300 Å column). Peak analysis was performed by auto-integrating using the following parameters: slope sensitivity=0.5, peak width=0, height reject=0, area reject=0, shoulders off, area percent reject 0, standard tangent skim mode, advanced baseline correction, 0 for front peak skim height ratio, 0 for tail peak skim height ratio, 0 for peak to valley ratio, and 0 for skim valley ratio.

Differential Scanning Fluorimetry (DSF) Measurements: The melting temperature of the protein before and after formulation, as well as at various time points of 40° C. storage, were assessed using a QuantStudio 6 Flex instrument. Five microliters (5 μL) of samples (1 mg/mL), prepared after dialysis, were loaded onto a 96-well thermal cycler plate in quadruplicate. To each well, 12.5 μL of ultrapure deionized water and 2.5 μL of SYPRO® Orange dye (8×) were added. After a 5-minute incubation, samples were run from 25° C. to 99° C. at a ramp rate of 0.05° C./s. Melting temperature was calculated using the Protein Thermal Shift Software (Thermo Fisher, version 1.3) using a Boltzmann fit.

Circular Dichroism (CD) Measurements: The degree of preservation of the secondary structure (alpha helices and beta sheets) of the protein before and after formulation, as well as at various time points of 40° C. storage, was assessed using a Jasco J-815 instrument. Four hundred microliters (400 μL) of sample (0.5 mg/mL), prepared after dialysis, was loaded into a quartz cuvette (1 mm path length). Samples were scanned over the 190-260 nm range. Diluent buffer was used as blank subtraction for each sample. The following instrument settings were typically used:

Photometric mode: CD, HT

Measure range: 260-190 nm

Data pitch: 0.5 nm

Sensitivity: Standard

D.I.T.: 4 sec

Bandwidth: 1.00 nm

Start mode: Immediately

Scanning speed: 100 nm/min

Shutter control: Auto

Baseline correction: None

CD detector: PMT

PMT voltage: Auto

Cation Exchange Chromatography (CIEX) Measurements: Charge variant analysis was performed for each sample on days 0, 7 and 30 under accelerated storage conditions, using an Agilent BioMAb NP5, 4.6×250 mm, PEEK ion exchange column. Samples were prepared at 1 mg/mL concentration after overnight dialysis in water. Buffer A was prepared with: 30 mM phosphate, pH: 6.3, and NaCl: 0 mM. Buffer B was prepared with: Buffer A: 30 mM phosphate, pH 6.3 plus NaCl: 175 mM. The samples were run in a gradient starting with 100% Buffer A, ramping up to a 100% Buffer B over a course of 20 min, after which the gradient was set to return to 100% Buffer A and 0% Buffer B in the next 1 min. The system re-equilibrated in 100% Buffer A for 10 min before the injection of the next sample. Integration was performed as a manual skim peak mode to reflect the Agilent data in the following protocol: https://www.agilent.com/cs/library/applications/5991-5557EN.pdf.

Monoclonal Antibody Binding Assay (Flow Cytometry): Monoclonal antibodies from select samples were assessed for cellular binding ability utilizing cells that express the appropriate cell surface receptors. Cells were incubated for 30 minutes at 4° C. with monoclonal antibodies at respective concentrations and then spun down at 2000 rpm followed by washing with PBS three times. Cells were then incubated with secondary goat anti-human Fab antibody (antigen-binding fragment) fluorescently labeled with PE for 30 minutes at 4° C. The cells were then spun down at 2000 rpm followed by washing with PBS, three times. The cells were then re-suspended and then analyzed on an Attune Flow Cytometer (Invitrogen). One million Raji cells (100 μL per well) were plated per well in a 96 well ‘V-bottom’ plate and 10 μL of mAb Label, particle, or suspension at a starting concentration of 200 μL was added to the wells. The dilution factor for the mAb label, particle and suspension was 3×. The plate was incubated at 4° C. for 30 min. The plate was centrifuged at 2000 rpm for 5 min and was washed 3 times with PBS. 100 μL of PE-conjugated goat anti-human IgG was added as the secondary antibody at a 1:200 dilution. The plate was centrifuged at 2000 rpm for 5 min and was washed 3 times with PBS. The cells were then resuspended in 200 μL of cold PBS for analysis on a Life Technologies Attune NXT flow cytometer. Alternatively, 1 Million Raji cells (100 μL per well) were plated per well in a 96 well ‘V-bottom’ plate and 10 μL of mAb, Label, particle, or suspension at a starting concentration of 200 μL was added to the wells. The dilution factor for the mAb label, particle and suspension was 3×. The plate was incubated at 4° C. for 30 min. The plate was centrifuged at 2000 rpm for 5 min and was washed 3 times with PBS. 100 μL of PE-conjugated goat anti-human IgG was added as the secondary antibody at a 1:200 dilution. The plate was centrifuged at 2000 rpm for 5 min and was washed 3 times with PBS. The cells were then resuspended in 200 μL of cold PBS for analysis on a Life Technologies Attune NXT flow cytometer.

Scanning Electron Microscopy (SEM): Electron micrographs were collected for select samples with either a Hitachi TM3030Plus or a TM1000 tabletop microscope. The samples were immobilized on conductive tape and examined in a low-vacuum anti-charging environment, obviating the need for sample preparation.

Image Analysis: Select microscopy images were chosen for further analysis on the basis of (i) minimal particle overlapping, (ii) good contrast between the particles and the background, and (iii) a resolution providing for particle occupancies of at least 10 pixels. This allowed for particles to be easily identified and reduced resolution-based error. A binary threshold was applied to separate the particles from background, and a watershed segmentation algorithm was applied to ensure that individual particles were measured separately. The ImageJ tool “Analyze Particles” was then applied on the binary picture with the following parameters: circularity between 0.5 and 1.0; size between 5 and infinity square microns; exclude on edges; fill holes. The outlines of the identified particles were overlaid onto the original image. Particles which were misidentified, such as clusters that were identified as a single particle or particles whose outlines do not match the particle, were then discarded. Missing particles were measured by manually tracing the particle's outline and using ImageJ's Measure tool.

Density Analysis: The skeletal density of particles from select samples was determined by examining approximately 0.1 g of powder with an AccuPyc II 1340 gas displacement pycnometry system.

Water Content Analysis: The residual moisture in particles from select samples was determined by placing approximately 0.01 g of powder in an oven with a Karl Fischer titrator and heating the sample.

Subvisible Particle (SvP) Analysis: Subvisible particles (SvPs) were analyzed with a Fluid Imaging Technologies FlowCam PV-100 system. Samples for analysis were reconstituted in sterile centrifuge tubes with filtered water (Milli-Q) to the concentration of interest. Three sets of samples were investigated thereafter. These included (i) a sample of the diluent used for reconstitution, (ii) an aliquot of the feed solution used for the particle formation process, i.e., a sample of the first liquid, and (iii) the reconstituted material.

Accelerated Storage: Storage was carried out under accelerated conditions for select samples by maintaining them at an elevated temperature (40° C.) for defined periods of time in an incubator or oven. Samples were kept in 2 mL or 4 mL Wheaton glass vials and sealed with paraffin film.

Helium Ion Microscopy (HIM): Ion micrographs were collected for select samples using an HIM instrument. The source energy, working distance, and aperture size were typically, 29 keV, 9 mm, and 10 microns, respectively. For select samples, a focused gallium ion beam was used to section particles for analysis of the internal structure. Tilted samples were ablated with a source current, dwell time, and cut spacing of 300 pA, 0.5-1 μs, and 2-5 nm, respectively.

X-Ray Photoelectron Spectroscopy (XPS): A small amount of powder was deposited onto hydrocarbon tape attached to a piece of silicon wafer and gently pressed to form a compact uniform bed. Excess loose powder was removed by lightly tapping the edge of the wafer piece. Specimens were prepared just before analysis. XPS measurements were performed with a Kratos Axis Ultra spectrometer using monochromatic A1 Kα X-rays (1486.6 eV). For each sample, a survey spectrum was acquired from an area of approximately 2 mm by 1 mm (pass energy=160 eV; 225 W power), from which the surface elemental composition was determined. Charge compensation was achieved using a beam of magnetically focused electrons as a flood current. The standard photoelectron take-off angle used for analysis is 90° giving a sampling depth in the range 5-8 nm. The surface elemental compositions were analyzed using a quantification model that assumes homogeneity of the probed sample volume.

Inverse Gas Chromatography (IGC): Powdered samples were analyzed using inverse gas chromatography. Cylindrical columns were packed with 200 to 300 mg of powdered samples to make up a stationary phase. Following an inert gas purge, a series of gas probes was injected on the column. Determination of the retention volume for each probe enabled evaluation of the dispersive and polar components of the surface energy for each sample.

X-Ray Diffraction (XRD): Samples were packed into 0.7 mm diameter glass capillaries. The powder patterns were measured on a PANalytical Empyrean diffractometer equipped with an incident-beam focusing mirror and an X'Celerator detector. The patterns (1-50° 2θ, 0.0167113° steps, 4 sec/step, ¼° divergence slit, 0.02 radian Soller slits) were measured using Mo Ká radiation. If static electricity effects (for the case of evaluating a lyophilization control this occurred after grinding in a mortar and pestle) prevented packing the sample into a capillary, its powder pattern was measured from a flat plate specimen on a Bruker D2 Phase diffractometer equipped with a LynxEye position-sensitive detector. The pattern was measured using Cu Kα radiation from 5-100° 20 in 0.0202144° steps, counting for 1.0 sec/step. The standard instrument settings (30 kV, 10 mA, 0.6 mm divergence slit, 2.5° Soller slits, and 3 mm scatter screen height) were employed.

Microflow Particle Sizing (MPS): Flow imaging microscopy for particle size analysis was carried out using a FlowCam PV-100. To investigate size and dispersity of particles, 5 mg of powder were dispersed in 10 mL of dry isopropanol via sonication. The isopropanol continuous phase prevented the particles from dissolving, i.e., prevented reconstitution. 0.3 mL was injected into the cell and images of the particles were taken using a flow rate of 0.15 mL/minute. Particles with a circularity greater than 0.9 were reported in the analysis and any double images were removed from the analysis, to give a size distribution and dispersity of particles in the range from 1 to 100 μm.

Dynamic Vapor Sorption (DVS): Powders were analyzed using dynamic water vapor sorption. Approximately 50 mg of powdered sample was loaded into the pan of the instrument's microbalance. The sample was held isothermally at 22° and the sample mass was monitored throughout the measurement. Following a 0% relative humidity (RH) purge to remove surface water, the RH in the sample chamber was ramped at a constant rate of 4% RH per hour up to 90% RH. The sample was held at 90% RH for one hour, then the RH was reduced to 0% as a step change. The sample was held at 0% RH for one hour, after which the measurement was terminated.

Dynamic Scanning calorimetry (DSC): Powdered samples were analyzed using dynamic scanning calorimetry. Masses of 5 to 10 mg of powdered samples were loaded into aluminum crucibles and sealed hermetically. Crucibles were loaded into the instrument, and the heat flow into the samples was monitored while the temperature was ramped from 30 to 250° C. at a constant rate of 5° C./minute.

USP <790>: According to the USP <790> standard, samples of dissolved particles were visually observed against a white and black background under lighting conditions greater than 2000 lux. Matte-finished high density polyethylene sheets were selected for the background to reduce glare. The illuminance at the viewing point was confirmed with a lux meter (Dr. Meter, LX1330B). The samples were swirled before being held up to the backgrounds and viewed for 5 sec.

Droplet Preparation. Preparation of droplets was accomplished by the following procedures, e.g., membrane emulsification, homogenization, mechanical stirring, mechanical shaking, impinging jet mixing, ultra-sound, sonication, micro-channel emulsification, microsieve emulsification, capillary extrusion, rotor-stator mixing, or static mixing. Prepare dispersed phase to be processed; remove any air or bubbles. This dispersed phase was introduced to the continuous phase via a range of methods depending on the droplet formation technique. In some cases, addition of the DP to the CP involved mixing (e.g. static mixing, micro-channel emulsification) and direct droplet formation. In other cases, the two phases, e.g., first liquid and second liquid, were added together but maintained phase separation until energy was applied and droplets could be formed (e.g. rotor stator mixing).

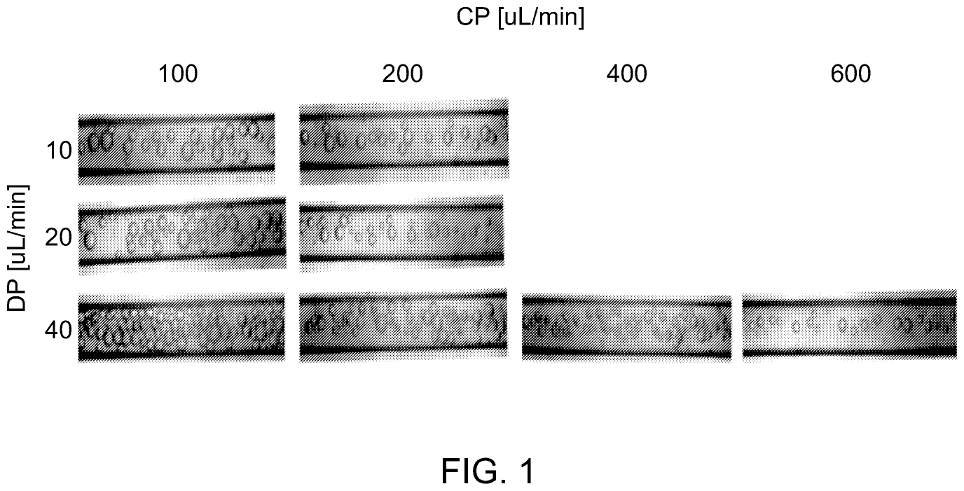

In a typical experiment 1 mL of dispersed phase was added to 100-200 mL of continuous phase to form droplets of around 1-80 microns as shown in FIG. 1. These droplets were stable over an intermediate timescale to allow for subsequent dehydration by a third liquid. Practitioners of the art will recognize that the order of certain steps in the below examples may be altered.

### EXAMPLES

The methods disclosed herein, have been utilized in separate instances to prepare particles including at least one of several therapeutic biologics, e.g., whole human IgG or bovine IgG, or one of several monoclonal antibodies, bovine serum albumin (BSA), or human serum albumin (HSA). Various analytical techniques were applied to assess the physical characteristics of the particles themselves as well as the structural and functional properties of the processed therapeutic biologics. Scanning electron microscopy and associated image analysis were used to study the particle morphology and size distribution, respectively. Various morphologies and distributions of components were achieved by controlling the properties of the first liquid, second liquid, and third liquid. In some instances, the processing conditions conferred smooth particles of high sphericity and/or facile control of the mean particle size over a broad range with low dispersity. Density and water content measurement demonstrated that the particles approached crystalline packing efficiencies and retained very low levels of residual moisture after post-processing. The functional properties of the therapeutic biologics were also preserved, as evidenced by ELISA and binding assays performed on reconstituted material. This was corroborated by size exclusion HPLC analysis indicating that the process had a minimal or even remedial effect on the degree of inter-antibody association. Finally, investigation of the insoluble particle populations upon reconstitution revealed very few insoluble artifacts, particularly as compared to alternative particle formation procedures.

### Example 1

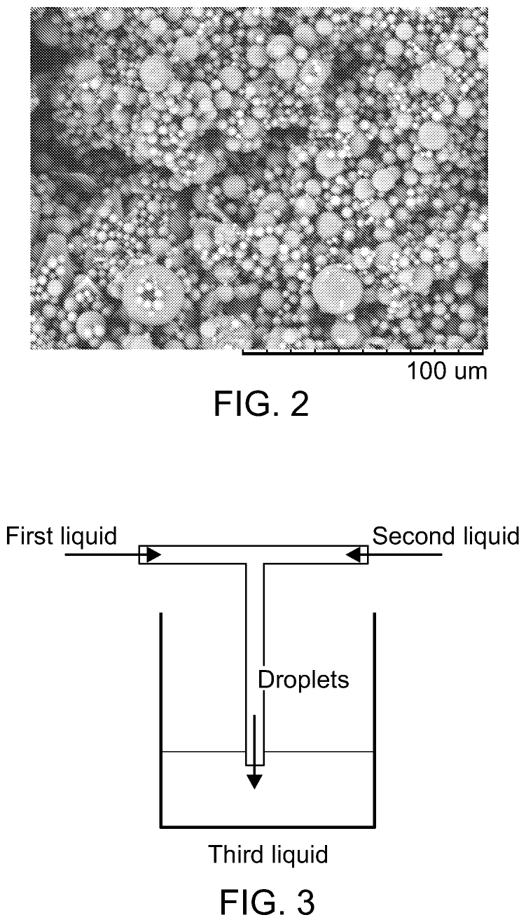

A PEEK Y connector assembly (0.04 inch) from IDEX (USA) was connected with tubing to a continuous phase (CP), a dispersed phase (DP) and an outlet tube. The dispersed phase (DP) was bovine serum albumin (BSA) prepared at 50 mg/mL in water. The continuous phase (CP) was a solution of heptane with 5 wt/vol % Span 80 surfactant. The DP and CP were mixed in the Y-connector assembly by pumping for 30 seconds at a flow rate of: DP=4.0 mL/min:CP=200 mL/min. The resulting water-in-oil emulsion was collected in a beaker. A second Y-assembly was set-up. Dehydration of the emulsion droplets was achieved by flowing the emulsion through one port of the Y-assembly. Through the other port butyl acetate was pumped. The flow rates used were: emulsion=10 mL/min:nBA=40 mL/min. This mixture was collected in a beaker with droplets dehydrating and forming particles. These particles were filtered and collected. SEM images revealed identifiable circular particulate matter at less than about 20 μm as shown in FIG. 2. The average circularity was calculated to be 0.915.

### Example 2

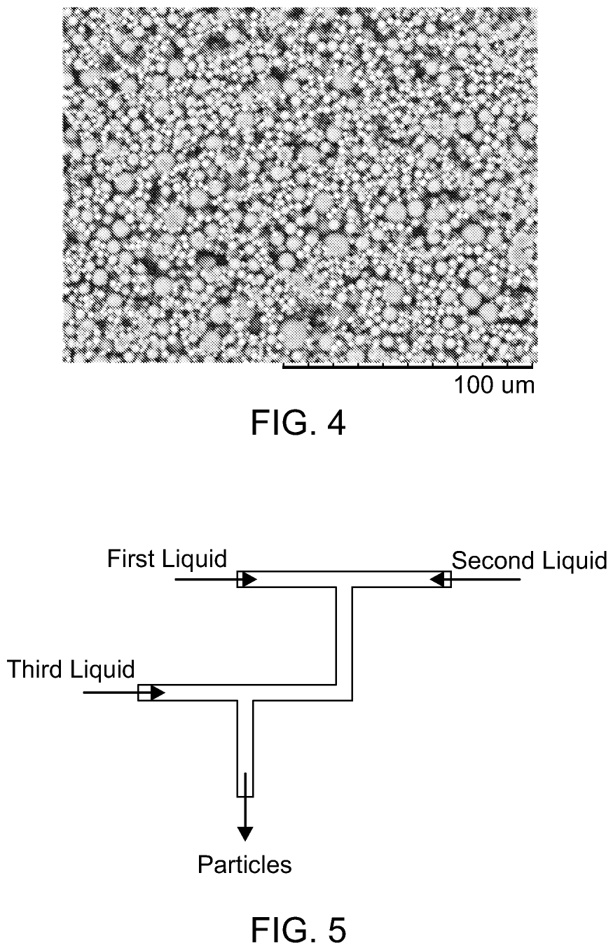

A PEEK Tee connector assembly (0.04 inch) from IDEX (USA) was connected with tubing to a continuous phase (CP), a dispersed phase (DP) and an outlet tube as shown in FIG. 3. The dispersed phase (DP) was bovine serum albumin (BSA) prepared at 50 mg/mL in water. The continuous phase (CP) was a solution of heptane with 5 wt/vol % Span 80 surfactant. The DP and CP were mixed in the Y-connector assembly by pumping for 2 minutes at a flow rate of: DP=0.5 mL/min:CP=200 mL/min. The resulting water-in-oil emulsion was collected in a beaker containing 500 mL of butyl acetate. Upon contact with the butyl acetate the droplets dehydrated and particles were formed. These particles were filtered and collected. SEM images revealed identifiable circular particulate matter at less than about 20 μm as shown in FIG. 4.

### Example 3

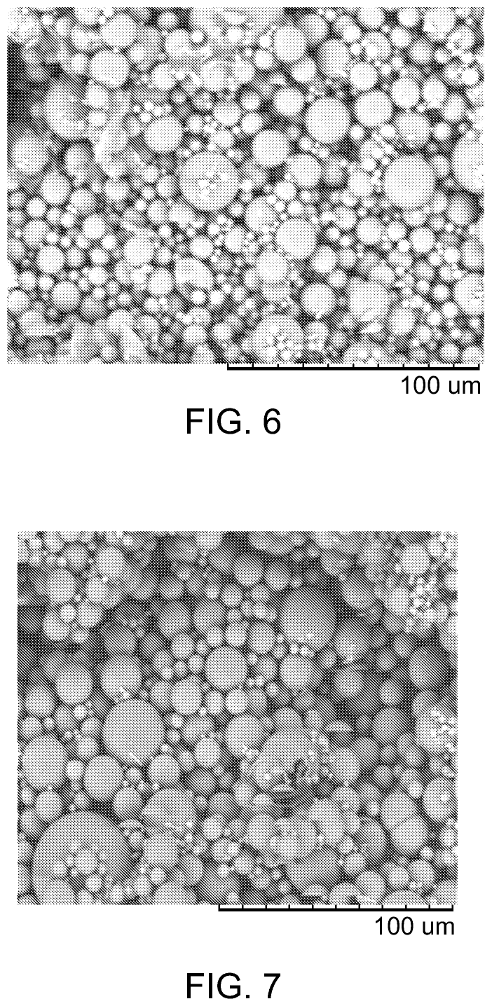

A PEEK Tee connector assembly (0.04 inch) from IDEX (USA) was connected with tubing to a continuous phase (CP), a dispersed phase (DP) and an outlet tube as shown in FIG. 5. The dispersed phase (DP) was a monoclonal antibody (mAb) prepared at 50 mg/mL in water. The continuous phase (CP) was a solution of heptane with 5 wt/vol % Span 80 surfactant. The DP and CP were mixed in the Y-connector assembly by pumping for 2 minutes at a flow rate of: DP=1.0 mL/min: CP=20 mL/min. The resulting water-in-oil emulsion flowed through the outlet to another Tee mixer. Through the other entry port of the Tee mixer flowed butyl acetate at 200 mL/min. Upon contact with the butyl acetate the droplets began to dehydrate and flowed out into a beaker. These particles were collected and dried in vacuo. Size exclusion chromatography (SEC) demonstrated that aggregate percentage increased by only 0.670 as compared to the feed solution. Subvisible particle (SvP) counts were around 250000 particles >1 micron at 1 mg/mL protein. And an increase in turbidity of around 0.41 (a.u.) as compared to the feed solution. SEM images revealed identifiable circular particulate matter at less than about 20 μm as shown in FIG. 6. The average circularity was calculated to be 0.924.

### Example 4

Example 4 was performed according to Example 3. The dispersed phase (DP) was BSA prepared at 50 mg/mL in water (0.5 mL/min). The continuous phase (CP) was a solution of heptane with 5 wt/vol % Span 80 surfactant (50 mL/min). Through the other entry port of the Tee mixer flowed butyl acetate at 100 mL/min. SEM images revealed identifiable circular particulate matter at less than about 30 μm as shown in FIG. 7. The average circularity was calculated to be 0.927.

### Example 5

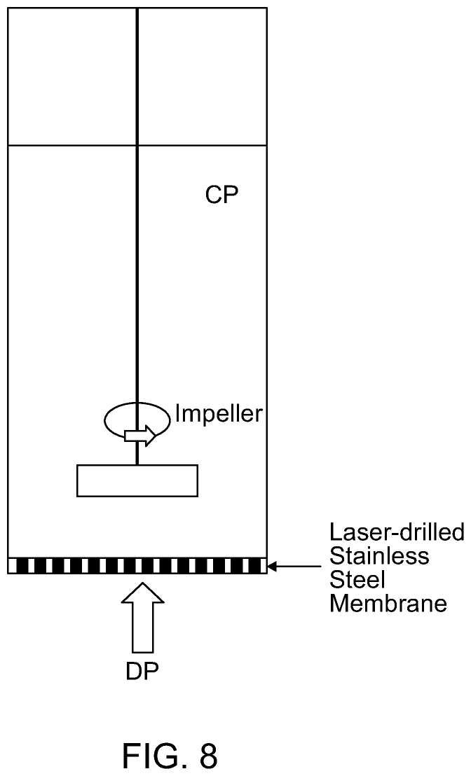

A general membrane emulsification system from Micropore Technologies is shown in FIG. 8 having a vessel containing a liquid continuous phase (CP) atop a laser-drilled stainless steel membrane, and a liquid dispersed phase (DP) that was pumped through the membrane was used to create droplets, which were detached by drag forces arising from the impeller rotation motion of the continuous phase. An exemplary component geometry of the unit is summarized in Table 2.

TABLE 2

| | Dimension |
|---|---|
| Feature | (mm) |
| Impeller diameter | 30.1 |
| Impeller height | 12.2 |
| Number of impeller blades | 2 |
| Vessel inner diameter of the threaded section | 34.1 |
| proximal to the membrane surface | |

Preparation of droplets was accomplished by the following exemplary procedure. Prepare a syringe with the dispersed phase to be processed; remove any air or bubbles. The dead volume is approximately 5 mL. Attach the injection tubing to the inlet barb of the system unit; attach the other end to the 3-way valve. Fill the injection chamber of the system unit with the continuous phase. Port a 10 mL syringe (priming syringe) into one branch of the 3-way valve and withdraw some of the continuous phase into the syringe, ensuring that all air bubbles have been removed from the system; leave some continuous phase in the bottom of the injection chamber. Top up the injection chamber with a sufficient volume to fully wet the membrane upon installation. Place the membrane atop the injection chamber not allowing the membrane to dry and ensure it is properly seated. Screw the glass cylinder into the injection chamber and tighten. Pour the target volume of continuous phase into the glass cylinder. Pull a small volume (˜0.5 mL) of CP back through the membrane into the priming syringe. Port the DP-containing syringe into the open branch of the 3-way valve. Adjust the position of the 3-way valve such that the DP syringe has an open path to the priming syringe and remove air bubbles. Adjust the 3-way valve position such that the DP syringe has an open path to the injection chamber. Install the DP syringe onto the syringe pump; set syringe diameter and flow rate. Install the stirrer motor assembly atop the glass cylinder; ensure the vortex breaker is positioned appropriately based on the volume of liquid to be processed. For example, 100 mL of CP and a stirring rate of 1200 rpm, the bottom of the vortex breaker rested about 4.5 cm above the top horizontal surface of the impeller. Adjust the stirrer voltage set point to reach the desired impeller rotation rate. Start infusing with the syringe pump, monitoring the supply line carefully for any signs of bubbles. Bubbles can be removed using the priming syringe if observed. Prime the system at a flow rate of 0.2 mL/min for 25 minutes followed by 0.1 mL/min for 3 minutes to replace the CP dead volume with DP. The amount to be injected can be controlled by adjusting the injection time based on the known DP flow rate. Once the required volume of DP has been injected, stop the syringe pump. Remove the stirrer motor assembly. Pour the emulsion into an appropriate receptacle for subsequent testing and analysis. Maintain stirring to keep the emulsion well-dispersed.

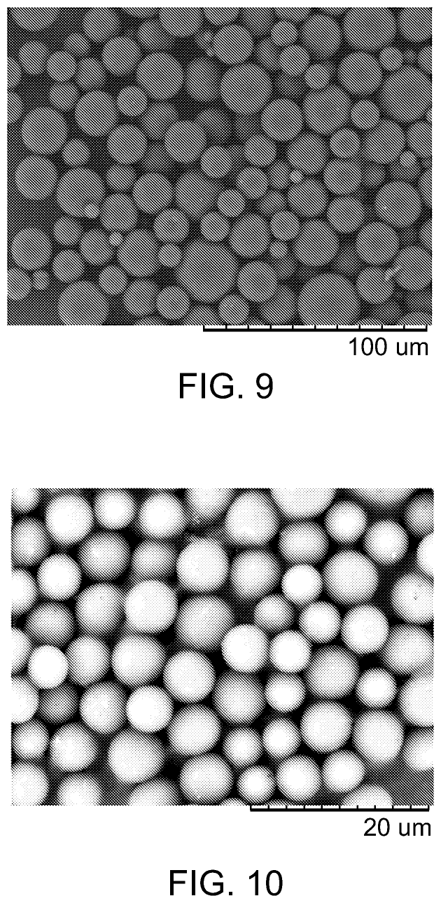

The emulsion was formed in n-heptane with 5 wt % Span 80 (CP) and 30 mg/mL BIgG solution (DP). The membrane pore size (5 μm), the impeller rotation rate (1200 rpm), the DP injection rate (0.1 mL/min) and volume (5.5 mL), and the CP volume (50 mL) provided well defined spherical droplets of the order of 10-20 μm with no droplet larger than 50 μm. The dehydration was carried out using a Y-shaped IDEX mixer with 200 mL nBA at a flow rate of 20 mL/min nBA+10 mL/min emulsion via Masterflex peristaltic pumps. The outlet of the Y was connected to a 0.45 μm nylon filter to separate the microparticles. These were subsequently washed with ˜200 mL ethyl acetate. SEM micrographs showed well defined, smooth circular particles at less than about 30 μm (FIG. 9). The average circularity was calculated to be 0.911.

### Example 6

Example 6 was performed according to Example 5. The emulsion was formed in n-heptane with 5 wt % Span 80 (CP) and 25 mg/mL BIgG solution (DP). The membrane pore size was 4 μm, the DP volume was 1 mL, and the CP volume was 100 mL provided the droplets. The dehydration was carried out with 200 mL of nBA. The particles were subsequently washed with 50 mL ×3 nBA. SEM micrographs showed well defined, smooth circular particles at less than about 10 μm (FIG. 10). The average circularity was calculated to be 0.850.

### Example 7

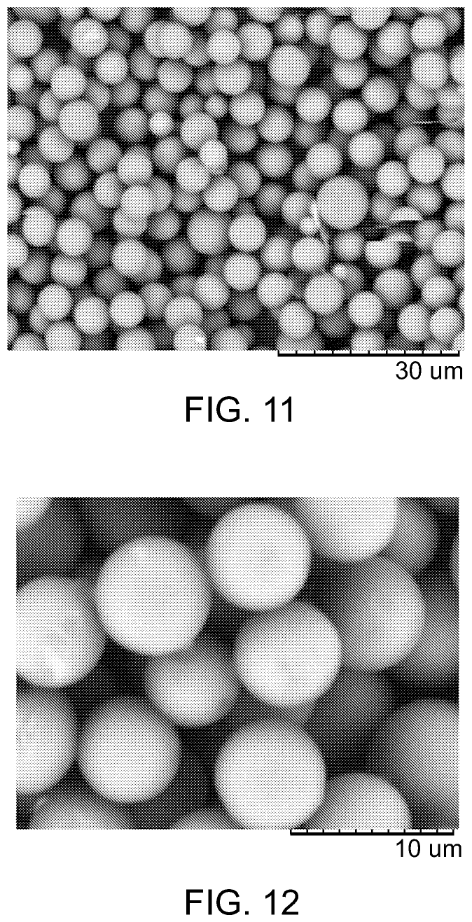

Example 7 was performed according to Example 5. The emulsion was formed in n-heptane with 5 wt % Span 80 (CP) and 50 mg/mL neat BSA (DP). The membrane pore size was 3 μm, the DP volume was 1 mL, and the CP volume was 100 mL provided the droplets. The dehydration was carried out with 200 mL of nBA. The particles were subsequently washed with 100 mL heptane. SEM micrographs showed well defined, smooth circular particles at less than about 10 μm (FIG. 11). The average circularity was calculated to be 0.852.

### Example 8

Example 8 was performed according to Example 5. The emulsion was formed in n-heptane with 5 wt % Span 80 (CP) and 51 mg/mL monoclonal antibody (DP). The membrane pore size was 3 μm, the DP volume was 1 mL, and the CP volume was 100 mL provided the droplets. The dehydration was carried out with 200 mL of nBA. The particles were subsequently washed with 100 mL heptane. SEM micrographs showed well defined, smooth circular particles at less than about 10 μm (FIG. 12). The average circularity was calculated to be 0.886.

### Example 9

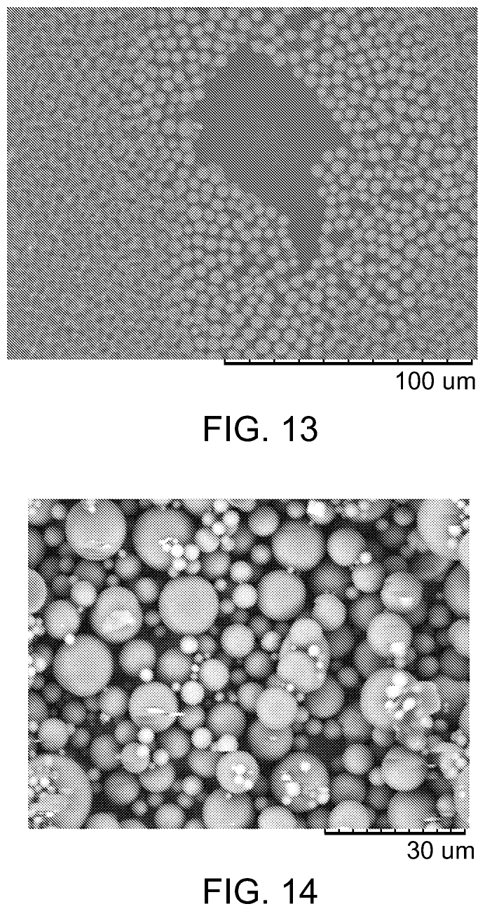

Example 9 was performed according to Example 5. The emulsion was formed in n-heptane with 5 wt % Span 80 (CP) and 25 mg/mL HIgG solution (DP). The DP volume was 1 mL, and the CP volume was 100 mL provided the droplets. The dehydration was carried out with nBA. The particles were subsequently washed with 100 mL nBA. SEM micrographs showed well defined, smooth circular particles at less than about 10 μm (FIG. 13).

### Example 10

An IKA T25 Easy Clean Digital rotor-stator mixer was equipped with an IKA 525-10G dispersion tool. 40 mL heptane was taken in a 50 mL Falcon conical centrifuge tube and the dispersion tool was immersed such that its tip was at the halfway mark of the heptane level in the tube. 0.4 mL of a solution of Human IgG was pipetted into the tube, and the mixer was run at a speed of 15000 rpm for 30 seconds. This resulted in an emulsion of the protein solution in heptane, which could be dehydrated by adding butyl acetate to the emulsion. SEM images revealed identifiable circular particulate matter at less than about 10 μm (FIG. 14). The average circularity was calculated to be 0.883.

### Example 11

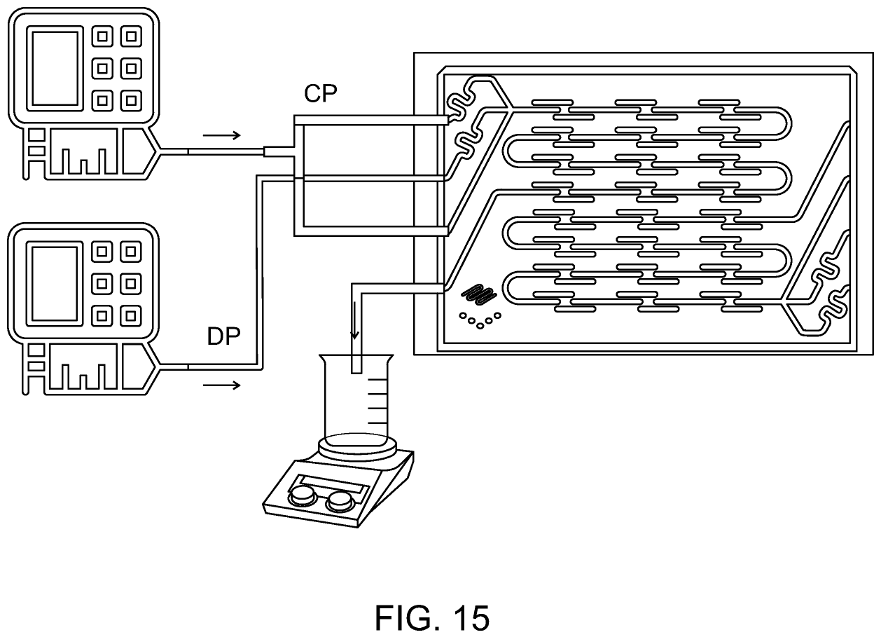

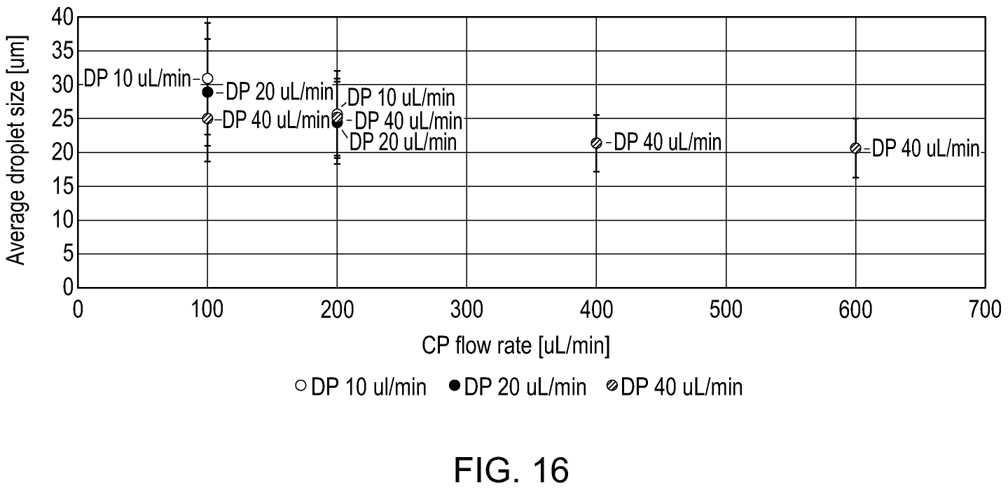

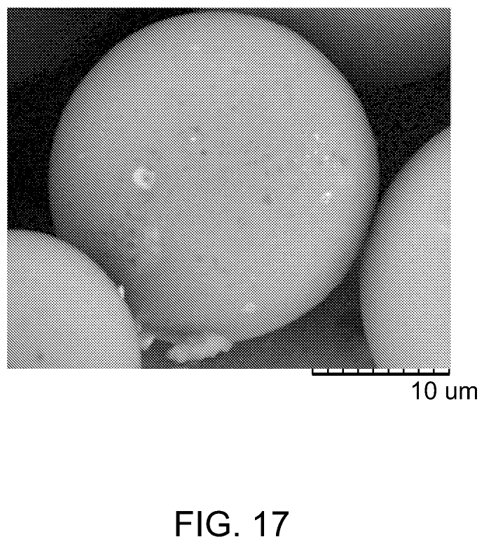

A Dolomite micromixer chip (part number 3200736) was used to generate droplets of protein therapeutics in heptane. These droplets in heptane were then combined with nButyl acetate to dehydrate the droplets into solid particles. The chip is set up with three inlets and one outlet as shown in FIG. 15. The disperse phase was comprised of a human IgG solution (25 mg/mL protein, 10 mM histidine, 8 mg/mL trehalose, 1% polysorbate 80) and was flowed through the central inlet at various flow rates. The continuous phase was comprised of heptane with 5% Span 80 and was flowed through the other two inlets at another set rate. The micromixing channels created droplets, which were collected from the outlet in a beaker. The droplets were imaged at the outlet of the device and had an average droplet size of about 20-30 μm as illustrated in FIG. 16. The emulsion was then flowed into a beaker at 10 mL/min while n-butylacetate was flowed into the same beaker at 20 mL/min. This allowed for the droplets to dehydrate into particles. The slurry was then pumped onto a filter membrane and the particles were collected on the filter membrane. The particles were dried under vacuum for 8 hours. SEM micrographs showed a smooth circular particle at 30 μm (FIG. 17).

### INCORPORATION BY REFERENCE

All publications and patents mentioned herein, are hereby incorporated by reference in their entirety as if each individual publication or patent was specifically and individually indicated to be incorporated by reference. In case of conflict, the present application, including any definitions herein, will control.

### EQUIVALENTS

While specific aspects and embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

1. A method of forming particles, the method comprising:
a) providing a first liquid comprising a therapeutic biologic and a solvent;
b) contacting the first liquid with a second liquid, thereby forming liquid droplets comprising the therapeutic biologic;
c) contacting the liquid droplets with a third liquid, thereby allowing the liquid droplets to dry; and
d) removing the first liquid, second liquid, and third liquid,
thereby forming particles comprising a therapeutic biologic, wherein the particles comprise less than about 10% internal void spaces and the circularity of the particles is from about 0.80 to about 1.00 after removing the first liquid, second liquid, and third liquid.

2. The method of claim 1, wherein the therapeutic biologic is an antibody, bovine serum albumin (BSA), or human serum albumin (HSA).

3. The method of claim 1, wherein the therapeutic biologic is an anti-CD20 antibody.

4. The method of claim 2 or 3, wherein the antibody is a monoclonal antibody.

5. The method of claim 2, 3 or 4, wherein the antibody is an IgG antibody.

6. The method of claim 5, wherein the IgG antibody is an IgG1 antibody.

7. The method of claim 6, wherein the IgG1 antibody is a monoclonal IgG1 antibody.

8. The method of any one of the preceding claims, wherein the therapeutic biologic in the particles has an activity per unit of about 0.5 to about 1.0.

9. The method of any one of the preceding claims, wherein the solvent is aqueous.

10. The method of claim 9, wherein the solvent is water, 0.9% saline, lactated Ringer's solution, a buffer, dextrose 5%, or a combination thereof.

11. The method of claim 10, wherein the solvent is water.

12. The method of claim 10, wherein the buffer is acetate buffer, histidine buffer, succinate buffer, HEPES buffer, tris buffer, carbonate buffer, citrate buffer, phosphate buffer, glycine buffer, barbital buffer, or cacodylate buffer.

13. The method of any one of the preceding claims, wherein the first liquid further comprises a carbohydrate, a pH adjusting agent, a salt, a chelator, a mineral, a polymer, a protein stabilizer, an emulsifier, an antiseptic, an amino acid, an antioxidant, a protein, an organic solvent, a paraben, a bactericide, a fungicide, a vitamin, a preservative, a nutrient media, an oligopeptide, a biologic excipient, a chemical excipient, a surfactant, or a combination thereof.

14. The method of claim 13, wherein the carbohydrate is dextran, trehalose, sucrose, agarose, mannitol, lactose, sorbitol, maltose, or a combination thereof.

15. The method of claim 13, wherein the pH adjusting agent is acetate, citrate, glutamate, glycinate, histidine, lactate, maleate, phosphate, succinate, tartrate, bicarbonate, aluminum hydroxide, phosphoric acid, hydrochloric acid, DL-lactic/glycolic acids, phosphorylethanolamine, tromethamine, imidazole, glyclyglycine, monosodium glutamate, sodium hydroxide, potassium hydroxide, or a combination thereof.

16. The method of claim 13, wherein the salt is sodium chloride, calcium chloride, potassium chloride, sodium hydroxide, stannous chloride, magnesium sulfate, sodium glucoheptonate, sodium pertechnetate, guanidine hydrochloride, potassium hydroxide, magnesium chloride, potassium nitrate, or a combination thereof.

17. The method of claim 13, wherein the protein stabilizer is trehalose, polyethylene glycol (PEG), polyoxamers, polyvinylpyrrolidone, polyacrylic acids, poly(vinyl) polymers, polyesters, polyaldehydes, tert-polymers, polyamino acids, hydroxyethylstarch, N-methyl-2-pyrrolidone, sorbitol, sucrose, mannitol, cyclodextrin, hydroxypropyl beta-cyclodextrin, sulfobutylether beta-cyclodextrin, or a combination thereof.

18. The method of claim 13, wherein the emulsifier is polysorbate, sorbitan monooleate, ethanolamine, polyoxyl 35 castor oil, poloxyl 40 hydrogenated castor oil, carbomer 1342, a corn oil-mono-di-triglyceride, a polyoxyethylated oleic glyceride, a poloxamer, or a combination thereof.

19. The method of claim 13, wherein the amino acid is alanine, aspartic acid, cysteine, isoleucine, glutamic acid, leucine, methionine, phenylalanine, pyrrolysine, serine, selenocysteine, threonine, tryptophan, tyrosine, valine, asparagine, arginine, histidine, glycine, glutamine, proline, or a combination thereof.

20. The method of claim 13, wherein the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, phosphatidylcholine, polyglycerol polyricinoleate, siloxanes, cetyl polyethylene glycol/polypropylene glycol-10/1 dimethicone triglyceride, bis-polyethylene glycol/polypropylene glycol-14/14 dimethicone, bis-(glyceryl/lauryl) glyceryl lauryl dimethicone (&) caprylic/capric triglyceride, cetyl polyethylene glycol/polypropylene glycol-10/1 dimethicone, phospholipids, or a combination thereof.

21. The method of claim 13, wherein the surfactant is polysorbate 80.

22. The method any one of the preceding claims, wherein the concentration of the therapeutic biologic in the first liquid is from about 0.0001 mg/mL to about 1000 mg/mL.

23. The method of claim 22, wherein the concentration of the therapeutic biologic in the first liquid is from about 10 mg/mL to about 500 mg/mL.

24. The method of claim 22 or 23, wherein the concentration of the therapeutic biologic in the first liquid is from about 10 mg/mL to about 100 mg/mL.

25. The method of any one of claims 22-24, wherein the concentration of the therapeutic biologic in the first liquid is from about 20 mg/mL to about 100 mg/mL.

26. The method of any one of the preceding claims, wherein the first liquid has a viscosity of less than about 100 mPa·s.

27. The method of claim 26, wherein the first liquid has a viscosity of less than about 10 mPa·s.

28. The method of claim 26 or 27, wherein the first liquid has a viscosity of less than about 3 mPa·s.

29. The method of any one of claims 26-28, wherein the first liquid has a viscosity of less than about 0.9 mPa·s.

30. The method of any one of the preceding claims, wherein the second liquid is an aqueous liquid, an organic solvent, an ionic liquid, a hydrogel, an ionogel, a protein stabilizer, or a combination thereof.

31. The method of claim 30, wherein the second liquid is an organic solvent.

32. The method of claim 31, wherein the organic solvent is acetonitrile, chlorobenzene, chloroform, cyclohexane, cumene, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, methylisobutylketone, N-methylpyrrolidone, nitromethane, pyridine, sulfolane, tetrahydrofuran, tetralin, toluene, 1,1,2-trichloroethene, xylene, acetic acid, acetone, anisole, 1-butanol, 2-butanol, butylacetate, tert-butylmethyl ether, dimethyl sulfoxide, ethanol, ethylacetate, ethyl ether, ethyl formate, formic acid, heptane, isobutylacetate, isopropylacetate, methylacetate, 3-methyl-1-butanol, methylethyl ketone, 2-methyl-1-propanol, pentane, 1-pentanol, 1-propanol, 2-propanol, propylacetate, triethylamine, 1,1-diethoxypropane, 1,1-dimethoxymethane, 2,2-dimethoxypropane, isooctane, isopropyl ether, methylisopropyl ketone, methyltetrahydrofuran, petroleum ether, trichloroacetic acid, trifluoroacetic acid, decanol, 2-ethylhexylacetate, amylacetate, or a combination thereof.

33. The method of claim 32, wherein the organic solvent is methylacetate, ethylacetate, propylacetate, butylacetate, amylacetate, 2-ethylhexylacetate, heptane, or a combination thereof.

34. The method of any one of the preceding claims, wherein the second liquid further comprises a surfactant.

35. The method of claim 34, wherein the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, phosphatidylcholine, polyglycerol polyricinoleate, siloxanes, cetyl polyethylene glycol/polypropylene glycol-10/1 dimethicone triglyceride, bis-polyethylene glycol/polypropylene glycol-14/14 dimethicone, bis-(glyceryl/lauryl) glyceryl lauryl dimethicone (&) caprylic/capric triglyceride, cetyl polyethylene glycol/polypropylene glycol-10/1 dimethicone, phospholipids, or a combination thereof.

36. The method of claim 34 or 35, wherein the surfactant is polysorbate 80.

37. The method of any one of the preceding claims, wherein the second liquid has a viscosity of less than about 50 mPa·s.

38. The method of claim 37, wherein the second liquid has a viscosity of less than about 10 mPa·s.

39. The method of claim 37 or 38, wherein the second liquid has a viscosity of less than about 5 mPa·s.

40. The method of any one of claims 37-39, wherein the second liquid has a viscosity of less than about 2 mPa·s.

41. The method of any one of claims 37-40, wherein the second liquid has a viscosity of less than about 0.40 mPa·s.

42. The method of any one of the preceding claims, wherein the liquid droplets of step b) are formed by membrane emulsification, homogenization, mechanical stirring, mechanical shaking, impinging jet mixing, ultra-sound, sonication, micro-channel emulsification, microsieve emulsification, capillary extrusion, static mixing, or a combination thereof.

43. The method of claim 42, wherein the liquid droplets of step b) are formed by membrane emulsification, homogenization, impinging jet mixing, static mixing, or a combination thereof.

44. The method of claim 42 or 43, wherein the membrane emulsification is conducted by rotating membrane emulsification, cross-flow membrane emulsification, or a combination thereof.

45. The method of claim 42 or 43, wherein the homogenization is conducted by shear homogenization, pressure homogenization, rotor-stator homogenization, microfluidization, or a combination thereof.

46. The method of claim 42, wherein the mechanical stirring is conducted by a turbulent stirred vessel, a magnetic stirring device, a mechanical stirring device, or a combination thereof.

47. The method of claim 42 or 43, wherein the static mixing comprises laminar flow, turbulent flow, transition flow, or a combination thereof.

48. The method of any one of the preceding claims, wherein the third liquid is an aqueous liquid, an organic solvent, an ionic liquid, a hydrogel, an ionogel, a protein stabilizer, or a combination thereof.

49. The method of claim 48, wherein the third liquid is an organic solvent.

50. The method of claim 49, wherein the organic solvent is acetonitrile, chlorobenzene, chloroform, cyclohexane, cumene, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, methylisobutylketone, N-methylpyrrolidone, nitromethane, pyridine, sulfolane, tetrahydrofuran, tetralin, toluene, 1,1,2-trichloroethene, xylene, acetic acid, acetone, anisole, 1-butanol, 2-butanol, butylacetate, tert-butylmethyl ether, dimethyl sulfoxide, ethanol, ethylacetate, ethyl ether, ethyl formate, formic acid, heptane, isobutylacetate, isopropylacetate, methylacetate, 3-methyl-1-butanol, methylethyl ketone, 2-methyl-1-propanol, pentane, 1-pentanol, 1-propanol, 2-propanol, propylacetate, triethylamine, 1,1-diethoxypropane, 1,1-dimethoxymethane, 2,2-dimethoxypropane, isooctane, isopropyl ether, methylisopropyl ketone, methyltetrahydrofuran, petroleum ether, trichloroacetic acid, trifluoroacetic acid, decanol, 2-ethylhexylacetate, amylacetate, or a combination thereof.

51. The method of claim 49 or 50, wherein the organic solvent is methylacetate, ethylacetate, propylacetate, butylacetate, amylacetate, 2-ethylhexylacetate, heptane, or a combination thereof.

52. The method of any one of the preceding claims, wherein the third liquid further comprises a surfactant.

53. The method of claim 52, wherein the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, phosphatidylcholine, polyglycerol polyricinoleate, siloxanes, cetyl polyethylene glycol/polypropylene glycol-10/1 dimethicone triglyceride, bis-polyethylene glycol/polypropylene glycol-14/14 dimethicone, bis-(glyceryl/lauryl) glyceryl lauryl dimethicone (&) caprylic/capric triglyceride, cetyl polyethylene glycol/polypropylene glycol-10/1 dimethicone, phospholipids, or a combination thereof.

54. The method of claim 52 or 53, wherein the surfactant is polysorbate 80.

55. The method of any one of the preceding claims, wherein the third liquid has a viscosity of less than about 50 mPa·s.

56. The method of claim 55, wherein the third liquid has a viscosity of less than about 10 mPa·s.

57. The method of claim 55 or 56, wherein the third liquid has a viscosity of less than about 5 mPa·s.

58. The method of any one of claims 55-57, wherein the third liquid has a viscosity of less than about 2 mPa·s.

59. The method of any one of claims 55-58, wherein the third liquid has a viscosity of less than about 0.40 mPa·s.

60. The method of any one of the preceding claims, wherein the liquid droplets of step c) are dried after contact with a third liquid.

61. The method of any one of the preceding claims, wherein step c) further comprises decreasing the temperature of the third liquid to a temperature within about 30° C. of the freezing point of the first liquid.

62. The method of any one of the preceding claims, wherein the boiling point of the third liquid at atmospheric pressure is from about 0 to about 200° C.

63. The method of any one of the preceding claims, wherein the first liquid, second liquid, and third liquid are removed through centrifugation, sieving, filtration, magnetic collection, solvent exchange, decanting, or a combination thereof.

64. The method of any one of the preceding claims, further comprising washing the particles after step d) with a washing fluid.

65. The method of claim 64, wherein the washing fluid is an organic liquid, a supercritical fluid, a cryogenic liquid, or a combination thereof.

66. The method of any one of the preceding claims, wherein the particles are further dried by lyophilization or vacuum desiccation.

67. The method of claim 66, wherein the particles are further dried by contacting the particles with a stream of gas.

68. The method of claim 67, wherein the gas has a temperature from about −80 to about 200° C.

69. The method of claim 67 or 68, wherein the gas has a temperature from about 10 to about 40° C.

70. The method of any one of claims 67-69, wherein the gas has a relative humidity greater than about 0% to less than about 100%.

71. The method of any one of claims 67-70, wherein the gas comprises helium, air, nitrogen or argon.

72. The method of any one of the preceding claims, further comprising sterilizing the particles after the first liquid, second liquid, and third liquid are removed.

73. The method of claim 72, wherein the sterilization occurs by irradiation, pasteurization, or freezing.

74. The method of claim 73, wherein the irradiation is by gamma radiation.

75. The method of any one of the preceding claims, wherein the particles comprise less than about 5% internal void spaces after removing the first liquid, second liquid, and third liquid.

76. The method of claim 75, wherein the particles comprise less than about 1% internal void spaces after removing the first liquid, second liquid, and third liquid.

77. The method of claim 75 or 76, wherein the particles comprise less than about 0.1% internal void spaces after removing the first liquid, second liquid, and third liquid.

78. The method of any one of claims 75-77, wherein the particles comprise less than about 0.01% internal void spaces after removing the first liquid, second liquid, and third liquid.

79. The method of any one of claims 75-78, wherein the particles are substantially free from any internal void spaces after removing the first liquid, second liquid, and third liquid.

80. The method of any one of the preceding claims, wherein the circularity of the particles is from about 0.85 to about 1.00 after removing the first liquid, second liquid, and third liquid.

81. The method of claim 80, wherein the circularity of the particles is from about 0.90 to about 1.00 after removing the first liquid, second liquid, and third liquid.

82. The method of claim 80 or 81, wherein the circularity of the particles is from about 0.95 to about 1.00 after removing the first liquid, second liquid, and third liquid.

83. The method of any one of claims 80-82, wherein the circularity of the particles is from about 0.98 to about 1.00 after removing the first liquid, second liquid, and third liquid.

84. The method of any one of claims 80-83, wherein the circularity of the particles is about 1.00 after removing the first liquid, second liquid, and third liquid.

85. The method of any one of the preceding claims, wherein the particles have a substantially smooth surface after removing the first liquid, second liquid, and third liquid.

86. The method of any one of the preceding claims, wherein the particles have a diameter of about 0.1 to about 1000 μm after removing the first liquid, second liquid, and third liquid.

87. The method of claim 86, wherein the particles have a diameter of about 1 to about 100 μm after removing the first liquid, second liquid, and third liquid.

88. The method of claim 86 or 87, wherein the particles have a diameter of about 5 to about 100 μm after removing the first liquid, second liquid, and third liquid.

89. The method of any one of claims 86-88, wherein the particles have a diameter of about 5 to about 50 μm after removing the first liquid, second liquid, and third liquid.

90. The method of any one of claims 86-89, wherein the particles have a diameter of about 5 to about 20 μm after removing the first liquid, second liquid, and third liquid.

91. The method of any one of the preceding claims, wherein the particles have a skeletal density of about 1.00 to about 6.00 g/cm³ after removing the first liquid, second liquid, and third liquid.

92. The method of claim 91, wherein the particles have a skeletal density of about 1.15 to about 1.60 g/cm³ after removing the first liquid, second liquid, and third liquid.

93. The method of claim 91 or 92, wherein the particles have a skeletal density of about 1.25 to about 1.50 g/cm³ after removing the first liquid, second liquid, and third liquid.

94. The method of any one of claims 91-93, wherein the particles have a skeletal density of about 1.30 to about 1.40 g/cm³ after removing the first liquid, second liquid, and third liquid.

95. The method of any one of the preceding claims, wherein the particles have a glass transition temperature that is higher than about 60° C. after removing the first liquid, second liquid, and third liquid.

96. The method of claim 95, wherein the particles have a glass transition temperature that is higher than about 90° C. after removing the first liquid, second liquid, and third liquid.

97. The method of claim 95 or 96, wherein the particles have a glass transition temperature that is higher than about 100° C. after removing the first liquid, second liquid, and third liquid.

98. The method of any one of claims 95-97, wherein the particles have a glass transition temperature that is higher than about 130° C. after removing the first liquid, second liquid, and third liquid.

99. The method of any one of claims 95-98, wherein the particles have a glass transition temperature that is higher than about 170° C. after removing the first liquid, second liquid, and third liquid.

100. The method of any one of the preceding claims, wherein the particles further comprise a carbohydrate, a pH adjusting agent, a salt, a chelator, a mineral, a polymer, a protein stabilizer, an emulsifier, an antiseptic, an amino acid, an antioxidant, a protein, an organic solvent, a paraben, a bactericide, a fungicide, a vitamin, a preservative, a nutrient media, an oligopeptide, a biologic excipient, a chemical excipient, a surfactant, or a combination thereof.

101. The method of claim 100, wherein the carbohydrate is dextran, trehalose, sucrose, agarose, mannitol, lactose, sorbitol, maltose, or a combination thereof.

102. The method of claim 100, wherein the pH adjusting agent is acetate, citrate, glutamate, glycinate, histidine, lactate, maleate, phosphate, succinate, tartrate, bicarbonate, aluminum hydroxide, phosphoric acid, hydrochloric acid, DL-lactic/glycolic acids, phosphorylethanolamine, tromethamine, imidazole, glyclyglycine, monosodium glutamate, sodium hydroxide, potassium hydroxide, or a combination thereof.

103. The method of claim 100, wherein the salt is sodium chloride, calcium chloride, potassium chloride, sodium hydroxide, stannous chloride, magnesium sulfate, sodium glucoheptonate, sodium pertechnetate, guanidine hydrochloride, potassium hydroxide, magnesium chloride, potassium nitrate, or a combination thereof.

104. The method of claim 100, wherein the protein stabilizer is trehalose, polyethylene glycol (PEG), polyoxamers, polyvinylpyrrolidone, polyacrylic acids, poly(vinyl) polymers, polyesters, polyaldehydes, tert-polymers, polyamino acids, hydroxyethylstarch, N-methyl-2-pyrrolidone, sorbitol, sucrose, mannitol, cyclodextrin, hydroxypropyl beta-cyclodextrin, sulfobutylether beta-cyclodextrin, or a combination thereof.

105. The method of claim 100, wherein the emulsifier is polysorbate, sorbitan monooleate, ethanolamine, polyoxyl 35 castor oil, poloxyl 40 hydrogenated castor oil, carbomer 1342, a corn oil-mono-di-triglyceride, a polyoxyethylated oleic glyceride, a poloxamer, or a combination thereof.

106. The method of claim 100, wherein the amino acid is alanine, aspartic acid, cysteine, isoleucine, glutamic acid, leucine, methionine, phenylalanine, pyrrolysine, serine, selenocysteine, threonine, tryptophan, tyrosine, valine, asparagine, arginine, histidine, glycine, glutamine, proline, or a combination thereof.

107. The method of claim 100, wherein the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, phosphatidylcholine, polyglycerol polyricinoleate, siloxanes, cetyl polyethylene glycol/polypropylene glycol-10/1 dimethicone triglyceride, bis-polyethylene glycol/polypropylene glycol-14/14 dimethicone, bis-(glyceryl/lauryl) glyceryl lauryl dimethicone (&) caprylic/capric triglyceride, cetyl polyethylene glycol/polypropylene glycol-10/1 dimethicone, phospholipids, or a combination thereof.

108. The method of claim 100, wherein the surfactant is polysorbate 80.

109. The method of any one of the preceding claims, wherein the particle has greater than about 60% therapeutic biologic by weight after removing the first liquid, second liquid, and third liquid.

110. The method of claim 109, wherein the particle has greater than about 70% therapeutic biologic by weight after removing the first liquid, second liquid, and third liquid.

111. The method of claim 109 or 110, wherein the particle has greater than about 80% therapeutic biologic by weight after removing the first liquid, second liquid, and third liquid.

112. The method of any one of claims 109-111, wherein the particle has greater than about 90% therapeutic biologic by weight after removing the first liquid, second liquid, and third liquid.

113. The method of any one of claims 109-112, wherein the particle has greater than about 95% therapeutic biologic by weight after removing the first liquid, second liquid, and third liquid.

114. The method of any one of claims 109-113, wherein the particle has greater than about 98% therapeutic biologic by weight after removing the first liquid, second liquid, and third liquid.

115. The method of any one of claims 109-114, wherein the particle has greater than about 99% therapeutic biologic by weight after removing the first liquid, second liquid, and third liquid.

116. The method of any one of the preceding claims, wherein the particles have less than about 10% aggregation of the therapeutic biologic after removing the first liquid, second liquid, and third liquid.

117. The method of claim 116, wherein the particles have less than about 5% aggregation of the therapeutic biologic after removing the first liquid, second liquid, and third liquid.

118. The method of claim 116 or 117, wherein the particles have less than about 3% aggregation of the therapeutic biologic after removing the first liquid, second liquid, and third liquid.

119. The method of any one of claims 116-118, wherein the particles have less than about 1% aggregation of the therapeutic biologic after removing the first liquid, second liquid, and third liquid.

120. The method of any one of claims 116-119, wherein the particles have less than about 0.5% aggregation of the therapeutic biologic after removing the first liquid, second liquid, and third liquid.

121. The method of any one of claims 116-120, wherein the particles are substantially free from any aggregation of the therapeutic biologic after removing the first liquid, second liquid, and third liquid.

122. The method of any one of claims 1-115, wherein the particles have about 3% to about 1% aggregation of the therapeutic biologic after removing the first liquid, second liquid, and third liquid.

123. The method of any one of claims 1-115, wherein the particles have about 1% to about 0.5% aggregation of the therapeutic biologic after removing the first liquid, second liquid, and third liquid.

124. The method of any one of the preceding claims, wherein the particles have less than about 10% fragmentation of the therapeutic biologic after removing the first liquid, second liquid, and third liquid.

125. The method of claim 124, wherein the particles have less than about 5% fragmentation of the therapeutic biologic after removing the first liquid, second liquid, and third liquid.

126. The method of claim 124 or 125, wherein the particles have less than about 3% fragmentation of the therapeutic biologic after removing the first liquid, second liquid, and third liquid.

127. The method of any one of claims 124-126, wherein the particles have less than about 1% fragmentation of the therapeutic biologic after removing the first liquid, second liquid, and third liquid.

128. The method of any one of claims 124-127, wherein the particles are substantially free from any fragmentation of the therapeutic biologic after removing the first liquid, second liquid, and third liquid.

129. The method of any one of the preceding claims, wherein the particles have less than about 50% change in charge variants of the therapeutic biologic after removing the first liquid, second liquid, and third liquid.

130. The method of claim 129, wherein the particles have less than about 30% change in charge variants of the therapeutic biologic after removing the first liquid, second liquid, and third liquid.

131. The method of claim 129 or 130, wherein the particles have less than about 25% change in charge variants of the therapeutic biologic after removing the first liquid, second liquid, and third liquid.

132. The method of any one of claims 129-131, wherein the particles have less than about 15% change in charge variants of the therapeutic biologic after removing the first liquid, second liquid, and third liquid.

133. The method of any one of claims 129-132, wherein the particles have less than about 10% change in charge variants of the therapeutic biologic after removing the first liquid, second liquid, and third liquid.

134. The method of any one of claims 129-133, wherein the particles have less than about 5% change in charge variants of the therapeutic biologic after removing the first liquid, second liquid, and third liquid.

135. The method of any one of claims 129-134, wherein the particles have less than about 3% change in charge variants of the therapeutic biologic after removing the first liquid, second liquid, and third liquid.

136. The method of any one of claims 129-135, wherein the particles have less than about 1% change in charge variants of the therapeutic biologic after removing the first liquid, second liquid, and third liquid.

137. The method of any one of claims 129-136, wherein the particles are substantially free from any change in charge variants of the therapeutic biologic after removing the first liquid, second liquid, and third liquid.

138. The method of any one of the preceding claims, wherein the particles have a surfactant content of less than about 10% by mass after removing the first liquid, second liquid, and third liquid.

139. The method of claim 138, wherein the particles have a surfactant content of less than about 5% by mass after removing the first liquid, second liquid, and third liquid.

140. The method of claim 138 or 139, wherein the particles have a surfactant content of less than about 3% by mass after removing the first liquid, second liquid, and third liquid.

141. The method of any one of claims 138-140, wherein the particles have a surfactant content of less than about 1% by mass after removing the first liquid, second liquid, and third liquid.

142. The method of any one of claims 138-141, wherein the particles have a surfactant content of less than about 0.1% by mass after removing the first liquid, second liquid, and third liquid.

143. The method of any one of claims 138-142, wherein the particles have a surfactant content of less than about 0.01% by mass after removing the first liquid, second liquid, and third liquid.

144. The method of any one of claims 138-143, wherein the particles have a surfactant content of less than about 0.001% by mass after removing the first liquid, second liquid, and third liquid.

145. The method of any one of claims 138-144, wherein the particles are substantially free from any surfactant content after removing the first liquid, second liquid, and third liquid.

146. The method of any one of the preceding claims, wherein the particles have less than about 3% of residual first liquid, second liquid, and third liquid by mass remaining after removing the first liquid, second liquid, and third liquid.

147. The method of claim 146, wherein the particles have less than about 2% of residual first liquid, second liquid, and third liquid by mass remaining after removing the first liquid, second liquid, and third liquid.

148. The method of claim 146 or 147, wherein the particles have less than about 1% of residual first liquid, second liquid, and third liquid by mass remaining after removing the first liquid, second liquid, and third liquid.

149. The method of any one of claims 146-148, wherein the particles have less than about 0.1% of residual first liquid, second liquid, and third liquid by mass remaining after removing the first liquid, second liquid, and third liquid.

150. The method of any one of claims 146-149, wherein the particles have less than about 0.01% of residual first liquid, second liquid, and third liquid by mass remaining after removing the first liquid, second liquid, and third liquid.

151. The method of any one of claims 146-150, wherein the particles are substantially free from any residual first liquid, second liquid, and third liquid by mass after removing the first liquid, second liquid, and third liquid.

152. The method of any one of the preceding claims, wherein the particles have less than about 5% of residual moisture by mass.

153. The method of claim 152, wherein the particles have less than about 3% of residual moisture by mass.

154. The method of claim 152 or 153, wherein the particles have less than about 2% of residual moisture by mass.

155. The method of any one of claims 152-154, wherein the particles have less than about 1% of residual moisture by mass.

156. The method of any one of claims 152-155, wherein the particles have less than about 0.1% of residual moisture by mass.

157. The method of any one of claims 152-156, wherein the particles have less than about 0.01% of residual moisture by mass.

158. The method of any one of claims 152-157, wherein the particles are substantially free from any residual moisture by mass.

159. A method of controlling the morphology of particles, the method comprising:
a) providing a first liquid comprising a therapeutic biologic and a solvent;
b) contacting the first liquid with a second liquid, thereby forming liquid droplets comprising the therapeutic biologic;
c) contacting the liquid droplets with a third liquid, thereby forming a mixture, wherein the Peclet number of the mixture determines the morphology of the particles;
d) allowing the liquid droplets to dry; and
e) removing the first liquid, second liquid, and third liquid,
thereby forming particles comprising a therapeutic biologic, wherein the particles comprise less than about 10% internal void spaces and the circularity of the particles is from about 0.80 to about 1.00 after removing the first liquid, second liquid, and third liquid.

160. The method of claim 159, wherein the therapeutic biologic is an antibody, bovine serum albumin (BSA), or human serum albumin (HSA).

161. The method of claim 159, wherein the therapeutic biologic is an anti-CD20 antibody.

162. The method of claim 160, wherein the antibody is monoclonal.

163. The method of claim 160, wherein the antibody is an IgG antibody.

164. The method of claim 163, wherein the IgG antibody is an IgG1 antibody.

165. The method of claim 164, wherein the IgG1 antibody is a monoclonal IgG1 antibody.

166. The method of any one of claims 159-165, wherein the therapeutic biologic in the particles has an activity per unit of about 0.5 to about 1.0.

167. The method of any one of claims 159-166, wherein the solvent is aqueous.

168. The method of claim 167, wherein the solvent is water, 0.9% saline, lactated Ringer's solution, a buffer, dextrose 5%, or a combination thereof.

169. The method of claim 168, wherein the solvent is water.

170. The method of claim 168, wherein the buffer is acetate buffer, histidine buffer, succinate buffer, HEPES buffer, tris buffer, carbonate buffer, citrate buffer, phosphate buffer, glycine buffer, barbital buffer, or cacodylate buffer.

171. The method of any one of claims 159-170, wherein the first liquid further comprises a carbohydrate, a pH adjusting agent, a salt, a chelator, a mineral, a polymer, a protein stabilizer, an emulsifier, an antiseptic, an amino acid, an antioxidant, a protein, an organic solvent, a paraben, a bactericide, a fungicide, a vitamin, a preservative, a nutrient media, an oligopeptide, a biologic excipient, a chemical excipient, a surfactant, or a combination thereof.

172. The method of claim 171, wherein the carbohydrate is dextran, trehalose, sucrose, agarose, mannitol, lactose, sorbitol, maltose, or a combination thereof.

173. The method of claim 171, wherein the pH adjusting agent is acetate, citrate, glutamate, glycinate, histidine, lactate, maleate, phosphate, succinate, tartrate, bicarbonate, aluminum hydroxide, phosphoric acid, hydrochloric acid, DL-lactic/glycolic acids, phosphorylethanolamine, tromethamine, imidazole, glyclyglycine, monosodium glutamate, sodium hydroxide, potassium hydroxide, or a combination thereof.

174. The method of claim 171, wherein the salt is sodium chloride, calcium chloride, potassium chloride, sodium hydroxide, stannous chloride, magnesium sulfate, sodium glucoheptonate, sodium pertechnetate, guanidine hydrochloride, potassium hydroxide, magnesium chloride, potassium nitrate, or a combination thereof.

175. The method of claim 171, wherein the protein stabilizer is trehalose, polyethylene glycol (PEG), polyoxamers, polyvinylpyrrolidone, polyacrylic acids, poly(vinyl) polymers, polyesters, polyaldehydes, tert-polymers, polyamino acids, hydroxyethylstarch, N-methyl-2-pyrrolidone, sorbitol, sucrose, mannitol, cyclodextrin, hydroxypropyl beta-cyclodextrin, sulfobutylether beta-cyclodextrin, or a combination thereof.

176. The method of claim 171, wherein the emulsifier is polysorbate, sorbitan monooleate, ethanolamine, polyoxyl 35 castor oil, poloxyl 40 hydrogenated castor oil, carbomer 1342, a corn oil-mono-di-triglyceride, a polyoxyethylated oleic glyceride, a poloxamer, or a combination thereof.

177. The method of claim 171, wherein the amino acid is alanine, aspartic acid, cysteine, isoleucine, glutamic acid, leucine, methionine, phenylalanine, pyrrolysine, serine, selenocysteine, threonine, tryptophan, tyrosine, valine, asparagine, arginine, histidine, glycine, glutamine, proline, or a combination thereof.

178. The method of claim 171, wherein the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, phosphatidylcholine, polyglycerol polyricinoleate, siloxanes, cetyl polyethylene glycol/polypropylene glycol-10/1 dimethicone triglyceride, bis-polyethylene glycol/polypropylene glycol-14/14 dimethicone, bis-(glyceryl/lauryl) glyceryl lauryl dimethicone (&) caprylic/capric triglyceride, cetyl polyethylene glycol/polypropylene glycol-10/1 dimethicone, phospholipids, or a combination thereof.

179. The method of claim 171, wherein the surfactant is polysorbate 80.

180. The method of any one of claims 159-179, wherein the concentration of the therapeutic biologic in the first liquid is from about 0.0001 mg/mL to about 1000 mg/mL.

181. The method of claim 180, wherein the concentration of the therapeutic biologic in the first liquid is from about 10 mg/mL to about 500 mg/mL.

182. The method of claim 180 or 181, wherein the concentration of the therapeutic biologic in the first liquid is from about 10 mg/mL to about 100 mg/mL.

183. The method of any one of claims 180-182, wherein the concentration of the therapeutic biologic in the first liquid is from about 20 mg/mL to about 100 mg/mL.

184. The method of any one of claims 159-183, wherein the first liquid has a viscosity of less than about 100 mPa·s.

185. The method of claim 184, wherein the first liquid has a viscosity of less than about 10 mPa·s.

186. The method of claim 184 or 185, wherein the first liquid has a viscosity of less than about 3 mPa·s.

187. The method of any one of claims 184-186, wherein the first liquid has a viscosity of less than about 0.9 mPa·s.

188. The method of any one of claims 159-183, wherein the second liquid is an aqueous liquid, an organic solvent, an ionic liquid, a hydrogel, ionogel, protein stabilizer, or a combination thereof.

189. The method of claim 188, wherein the second liquid is an organic solvent.

190. The method of claim 189, wherein the organic solvent is acetonitrile, chlorobenzene, chloroform, cyclohexane, cumene, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, methylisobutylketone, N-methylpyrrolidone, nitromethane, pyridine, sulfolane, tetrahydrofuran, tetralin, toluene, 1,1,2-trichloroethene, xylene, acetic acid, acetone, anisole, 1-butanol, 2-butanol, butylacetate, tert-butylmethyl ether, dimethyl sulfoxide, ethanol, ethylacetate, ethyl ether, ethyl formate, formic acid, heptane, isobutylacetate, isopropylacetate, methylacetate, 3-methyl-1-butanol, methylethyl ketone, 2-methyl-1-propanol, pentane, 1-pentanol, 1-propanol, 2-propanol, propylacetate, triethylamine, 1,1-diethoxypropane, 1,1-dimethoxymethane, 2,2-dimethoxypropane, isooctane, isopropyl ether, methylisopropyl ketone, methyltetrahydrofuran, petroleum ether, trichloroacetic acid, trifluoroacetic acid, decanol, 2-ethylhexylacetate, amylacetate, or a combination thereof.

191. The method of claim 189 or 190, wherein the organic solvent is methylacetate, ethylacetate, propylacetate, butylacetate, amylacetate, 2-ethylhexylacetate, heptane, or a combination thereof.

192. The method of any one of claims 159-191, wherein the second liquid further comprises a surfactant.

193. The method of claim 192, wherein the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, phosphatidylcholine, polyglycerol polyricinoleate, siloxanes, cetyl polyethylene glycol/polypropylene glycol-10/1 dimethicone triglyceride, bis-polyethylene glycol/polypropylene glycol-14/14 dimethicone, bis-(glyceryl/lauryl) glyceryl lauryl dimethicone (&) caprylic/capric triglyceride, cetyl polyethylene glycol/polypropylene glycol-10/1 dimethicone, phospholipids, or a combination thereof.

194. The method of claim 192 or 193, wherein the surfactant is polysorbate 80.

195. The method of any one of claims 159-194, wherein the second liquid has a viscosity of less than about 50 mPa·s.

196. The method of claim 195, wherein the second liquid has a viscosity of less than about 10 mPa·s.

197. The method of claim 195 or 196, wherein the second liquid has a viscosity of less than about 5 mPa·s.

198. The method of any one of claims 195-197, wherein the second liquid has a viscosity of less than about 2 mPa·s.

199. The method of any one of claims 195-198, wherein the second liquid has a viscosity of less than about 0.40 mPa·s.

200. The method of any one of claims 159-199, wherein the liquid droplets of step b) are formed by membrane emulsification, homogenization, mechanical stirring, mechanical shaking, impinging jet mixing, ultra-sound, sonication, micro-channel emulsification, microsieve emulsification, capillary extrusion, static mixing, or a combination thereof.

201. The method of claim 200, wherein the liquid droplets of step b) are formed by membrane emulsification homogenization, impinging jet mixing, static mixing, or a combination thereof.

202. The method of claim 200 or 201, wherein the membrane emulsification is conducted by rotating membrane emulsification, cross-flow membrane emulsification, or a combination thereof.

203. The method of claim 200 or 201, wherein the homogenization is conducted by shear homogenization, pressure homogenization, rotor-stator homogenization, microfluidization, or a combination thereof.

204. The method of claim 200, wherein the mechanical stirring is conducted by a turbulent stirred vessel, a magnetic stirring device, a mechanical stirring device, or a combination thereof.

205. The method of claim 200 or 201, wherein the static mixing comprises laminar flow, turbulent flow, transition flow, or a combination thereof.

206. The method of any one of claims 159-205, wherein the third liquid is an aqueous liquid, an organic solvent, an ionic liquid, a hydrogel, ionogel, protein stabilizer, or a combination thereof.

207. The method of claim 206, wherein the third liquid is an organic solvent.

208. The method of claim 206, wherein the organic solvent is acetonitrile, chlorobenzene, chloroform, cyclohexane, cumene, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, methylisobutylketone, N-methylpyrrolidone, nitromethane, pyridine, sulfolane, tetrahydrofuran, tetralin, toluene, 1,1,2-trichloroethene, xylene, acetic acid, acetone, anisole, 1-butanol, 2-butanol, butylacetate, tert-butylmethyl ether, dimethyl sulfoxide, ethanol, ethylacetate, ethyl ether, ethyl formate, formic acid, heptane, isobutylacetate, isopropylacetate, methylacetate, 3-methyl-1-butanol, methylethyl ketone, 2-methyl-1-propanol, pentane, 1-pentanol, 1-propanol, 2-propanol, propylacetate, triethylamine, 1,1-diethoxypropane, 1,1-dimethoxymethane, 2,2-dimethoxypropane, isooctane, isopropyl ether, methylisopropyl ketone, methyltetrahydrofuran, petroleum ether, trichloroacetic acid, trifluoroacetic acid, decanol, 2-ethylhexylacetate, amylacetate, or a combination thereof.

209. The method of claim 206 or 207, wherein the organic solvent is methylacetate, ethylacetate, propylacetate, butylacetate, amylacetate, 2-ethylhexylacetate, heptane, or a combination thereof.

210. The method of any one of claims 159-209, wherein the third liquid further comprises a surfactant.

211. The method of claim 210, wherein the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, phosphatidylcholine, polyglycerol polyricinoleate, siloxanes, cetyl polyethylene glycol/polypropylene glycol-10/1 dimethicone triglyceride, bis-polyethylene glycol/polypropylene glycol-14/14 dimethicone, bis-(glyceryl/lauryl) glyceryl lauryl dimethicone (&) caprylic/capric triglyceride, cetyl polyethylene glycol/polypropylene glycol-10/1 dimethicone, phospholipids, or a combination thereof.

212. The method of claim 210 or 211, wherein the surfactant is polysorbate 80.

213. The method of any one of claims 159-212, wherein the third liquid has a viscosity of less than about 50 mPa·s.

214. The method of claim 213, wherein the third liquid has a viscosity of less than about 10 mPa·s.

215. The method of claim 213 or 214, wherein the third liquid has a viscosity of less than about 5 mPa·s.

216. The method of any one of claims 213-215, wherein the third liquid has a viscosity of less than about 2 mPa·s.

217. The method of any one of claims 213-216, wherein the third liquid has a viscosity of less than about 0.40 mPa·s.

218. The method of any one of claims 159-217, wherein the Peclet number of the mixture of step c) is less than about 500.

219. The method of claim 218, wherein the Peclet number of the mixture of step c) is less than about 10.

220. The method of claim 218 or 219, wherein the Peclet number of the mixture of step c) is less than about 5.

221. The method of any one of claims 218-220, wherein the Peclet number of the mixture of step c) is less than about 3.

222. The method of any one of claims 218-221, wherein the Peclet number of the mixture of step c) is less than about 2.

223. The method of any one of claims 218-222, wherein the Peclet number of the mixture of step c) is less than about 1.

224. The method of any one of claims 159-223, wherein the liquid droplets of step c) are dried after contact with a third liquid.

225. The method of any one of claims 159-224, wherein step c) further comprises decreasing the temperature of the third liquid to a temperature within about 30° C. of the freezing point of the first liquid.

226. The method of any one of claims 159-225, wherein the boiling point of the third liquid at atmospheric pressure is from about 0 to about 200° C.

227. The method of any one of claims 159-226, wherein the first liquid, second liquid, and third liquid is removed through centrifugation, sieving, filtration, magnetic collection, solvent exchange, decanting, or a combination thereof.

228. The method of any one of claims 159-227, further comprising washing the particles after step e) with a washing fluid.

229. The method of claim 228, wherein the washing fluid is an organic liquid, a supercritical fluid, a cryogenic liquid, or a combination thereof.

230. The method of any one of claims 159-229, wherein the particles are further dried by lyophilization or vacuum desiccation.

231. The method of claim 230, wherein the particles are further dried by contacting the particles with a stream of gas.

232. The method of claim 231, wherein the gas has a temperature from about −80 to about 200° C.

233. The method of claim 231 or 232, wherein the gas has a temperature from about 10 to about 40° C.

234. The method of any one of claims 231-233, wherein the gas has a relative humidity greater than about 0% to less than about 100%.

235. The method of any one of claims 231-234, wherein the gas comprises helium, air, nitrogen or argon.

236. The method of any one of claims 159-235, further comprises sterilization of the particles after the first liquid, second liquid, and third liquid is removed.

237. The method of claim 236, wherein the sterilization occurs by irradiation, pasteurization, or freezing.

238. The method of claim 237, wherein the irradiation is by gamma radiation.

239. The method of any one of claims 159-238, wherein the particles comprise less than about 5% internal void spaces after removing the first liquid, second liquid, and third liquid.

240. The method of claim 239, wherein the particles comprise less than about 1% internal void spaces after removing the first liquid, second liquid, and third liquid.

241. The method of claim 239 or 240, wherein the particles comprise less than about 0.1% internal void spaces after removing the first liquid, second liquid, and third liquid.

242. The method of any one of claims 239-241, wherein the particles comprise less than about 0.01% internal void spaces after removing the first liquid, second liquid, and third liquid.

243. The method of any one of claims 239-242, wherein the particles are substantially free from any internal void spaces after removing the first liquid, second liquid, and third liquid.

244. The method of any one of claims 159-243, wherein the circularity of the particles is from about 0.85 to about 1.00 after removing the first liquid, second liquid, and third liquid.

245. The method of claim 244, wherein the circularity of the particles is from about 0.90 to about 1.00 after removing the first liquid, second liquid, and third liquid.

246. The method of claim 244 or 245, wherein the circularity of the particles is from about 0.95 to about 1.00 after removing the first liquid, second liquid, and third liquid.

247. The method of any one of claims 244-246, wherein the circularity of the particles is from about 0.98 to about 1.00 after removing the first liquid, second liquid, and third liquid.

248. The method of any one of claims 244-247, wherein the circularity of the particles is about 1.00 after removing the first liquid, second liquid, and third liquid.

249. The method of any one of claims 159-248, wherein the particles have a substantially smooth surface after removing the first liquid, second liquid, and third liquid.

250. The method of any one of claims 159-249, wherein the particles have a diameter of about 0.1 to about 1000 μm after removing the first liquid, second liquid, and third liquid.

251. The method of claim 250, wherein the particles have a diameter of about 1 to about 100 μm after removing the first liquid, second liquid, and third liquid.

252. The method of claim 250 or 251, wherein the particles have a diameter of about 5 to about 100 μm after removing the first liquid, second liquid, and third liquid.

253. The method of any one of claims 250-252, wherein the particles have a diameter of about 5 to about 50 μm after removing the first liquid, second liquid, and third liquid.

254. The method of any one of claims 250-253, wherein the particles have a diameter of about 5 to about 20 μm after removing the first liquid, second liquid, and third liquid.

255. The method of any one of claims 159-254, wherein the particles have a skeletal density of about 1.00 to about 6.00 g/cm³ after removing the first liquid, second liquid, and third liquid.

256. The method of claim 255, wherein the particles have a skeletal density of about 1.15 to about 1.60 g/cm³ after removing the first liquid, second liquid, and third liquid.

257. The method of claim 255 or 256, wherein the particles have a skeletal density of about 1.25 to about 1.50 g/cm³ after removing the first liquid, second liquid, and third liquid.

258. The method of any one of claims 255-257, wherein the particles have a skeletal density of about 1.30 to about 1.40 g/cm³ after removing the first liquid, second liquid, and third liquid.

259. The method of any one of claims 159-258, wherein the particles have a glass transition temperature that is higher than about 60° C. after removing the first liquid, second liquid, and third liquid.

260. The method of claim 259, wherein the particles have a glass transition temperature that is higher than about 90° C. after removing the first liquid, second liquid, and third liquid.

261. The method of claim 259 or 260, wherein the particles have a glass transition temperature that is higher than about 100° C. after removing the first liquid, second liquid, and third liquid.

262. The method of any one of claims 159-258, wherein the particles have a glass transition temperature that is higher than about 130° C. after removing the first liquid, second liquid, and third liquid.

263. The method of any one of claims 159-258, wherein the particles have a glass transition temperature that is higher than about 170° C. after removing the first liquid, second liquid, and third liquid.

264. The method of any one of claims 159-263, wherein the particles further comprise a carbohydrate, a pH adjusting agent, a salt, a chelator, a mineral, a polymer, a protein stabilizer, an emulsifier, an antiseptic, an amino acid, an antioxidant, a protein, an organic solvent, a paraben, a bactericide, a fungicide, a vitamin, a preservative, a nutrient media, an oligopeptide, a biologic excipient, a chemical excipient, a surfactant, or a combination thereof.

265. The method of claim 264, wherein the carbohydrate is dextran, trehalose, sucrose, agarose, mannitol, lactose, sorbitol, maltose, or a combination thereof.

266. The method of claim 264, wherein the pH adjusting agent is acetate, citrate, glutamate, glycinate, histidine, lactate, maleate, phosphate, succinate, tartrate, bicarbonate, aluminum hydroxide, phosphoric acid, hydrochloric acid, DL-lactic/glycolic acids, phosphorylethanolamine, tromethamine, imidazole, glyclyglycine, monosodium glutamate, sodium hydroxide, potassium hydroxide, or a combination thereof.

267. The method of claim 264, wherein the salt is sodium chloride, calcium chloride, potassium chloride, sodium hydroxide, stannous chloride, magnesium sulfate, sodium glucoheptonate, sodium pertechnetate, guanidine hydrochloride, potassium hydroxide, magnesium chloride, potassium nitrate, or a combination thereof.

268. The method of claim 264, wherein the protein stabilizer is trehalose, polyethylene glycol (PEG), polyoxamers, polyvinylpyrrolidone, polyacrylic acids, poly(vinyl) polymers, polyesters, polyaldehydes, tert-polymers, polyamino acids, hydroxyethylstarch, N-methyl-2-pyrrolidone, sorbitol, sucrose, mannitol, cyclodextrin, hydroxypropyl beta-cyclodextrin, sulfobutylether beta-cyclodextrin, or a combination thereof.

269. The method of claim 264, wherein the emulsifier is polysorbate, sorbitan monooleate, ethanolamine, polyoxyl 35 castor oil, poloxyl 40 hydrogenated castor oil, carbomer 1342, a corn oil-mono-di-triglyceride, a polyoxyethylated oleic glyceride, a poloxamer, or a combination thereof.

270. The method of claim 264, wherein the amino acid is alanine, aspartic acid, cysteine, isoleucine, glutamic acid, leucine, methionine, phenylalanine, pyrrolysine, serine, selenocysteine, threonine, tryptophan, tyrosine, valine, asparagine, arginine, histidine, glycine, glutamine, proline, or a combination thereof.

271. The method of claim 264, wherein the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, phosphatidylcholine, polyglycerol polyricinoleate, siloxanes, cetyl polyethylene glycol/polypropylene glycol-10/1 dimethicone triglyceride, bis-polyethylene glycol/polypropylene glycol-14/14 dimethicone, bis-(glyceryl/lauryl) glyceryl lauryl dimethicone (&) caprylic/capric triglyceride, cetyl polyethylene glycol/polypropylene glycol-10/1 dimethicone, phospholipids, or a combination thereof.

272. The method of claim 264, wherein the surfactant is polysorbate 80.

273. The method of any one of claims 159-272, wherein the particle has greater than about 60% therapeutic biologic by weight after removing the first liquid, second liquid, and third liquid.

274. The method of claim 273, wherein the particle has greater than about 70% therapeutic biologic by weight after removing the first liquid, second liquid, and third liquid.

275. The method of claim 273 or 274, wherein the particle has greater than about 80% therapeutic biologic by weight after removing the first liquid, second liquid, and third liquid.

276. The method of any one of claims 273-275, wherein the particle has greater than about 90% therapeutic biologic by weight after removing the first liquid, second liquid, and third liquid.

277. The method of any one of claims 273-276, wherein the particle has greater than about 95% therapeutic biologic by weight after removing the first liquid, second liquid, and third liquid.

278. The method of any one of claims 273-277, wherein the particle has greater than about 98% therapeutic biologic by weight after removing the first liquid, second liquid, and third liquid.

279. The method of any one of claims 273-278, wherein the particle has greater than about 99% therapeutic biologic by weight after removing the first liquid, second liquid, and third liquid.

280. The method of any one of claims 159-279, wherein the particles have less than about 10% aggregation of the therapeutic biologic after removing the first liquid, second liquid, and third liquid.

281. The method of claim 280, wherein the particles have less than about 5% aggregation of the therapeutic biologic after removing the first liquid, second liquid, and third liquid.

282. The method of claim 280 or 281, wherein the particles have less than about 3% aggregation of the therapeutic biologic after removing the first liquid, second liquid, and third liquid.

283. The method of any one of claims 280-282, wherein the particles have less than about 1% aggregation of the therapeutic biologic after removing the first liquid, second liquid, and third liquid.

284. The method of any one of claims 280-283, wherein the particles have less than about 0.5% aggregation of the therapeutic biologic after removing the first liquid, second liquid, and third liquid.

285. The method of any one of claims 280-284, wherein the particles are substantially free from any aggregation of the therapeutic biologic after removing the first liquid, second liquid, and third liquid.

286. The method of any one of claims 159-279, wherein the particles have about 3% to about 1% aggregation of the therapeutic biologic after removing the first liquid, second liquid, and third liquid.

287. The method of any one of claims 159-279, wherein the particles have about 1% to about 0.5% aggregation of the therapeutic biologic after removing the first liquid, second liquid, and third liquid.

288. The method of any one of claims 159-287, wherein the particles have less than about 10% fragmentation of the therapeutic biologic after removing the first liquid, second liquid, and third liquid.

289. The method of claim 288, wherein the particles have less than about 5% fragmentation of the therapeutic biologic after removing the first liquid, second liquid, and third liquid.

290. The method of claim 288 or 289, wherein the particles have less than about 3% fragmentation of the therapeutic biologic after removing the first liquid, second liquid, and third liquid.

291. The method of any one of claims 288-290, wherein the particles have less than about 1% fragmentation of the therapeutic biologic after removing the first liquid, second liquid, and third liquid.

292. The method of any one of claims 288-291, wherein the particles are substantially free from any fragmentation of the therapeutic biologic after removing the first liquid, second liquid, and third liquid.

293. The method of any one of claims 159-292, wherein the particles have less than about 50% change in charge variants of the therapeutic biologic after removing the first liquid, second liquid, and third liquid.

294. The method of claim 293, wherein the particles have less than about 30% change in charge variants of the therapeutic biologic after removing the first liquid, second liquid, and third liquid.

295. The method of claim 293 or 294, wherein the particles have less than about 25% change in charge variants of the therapeutic biologic after removing the first liquid, second liquid, and third liquid.

296. The method of any one of claims 293-295, wherein the particles have less than about 15% change in charge variants of the therapeutic biologic after removing the first liquid, second liquid, and third liquid.

297. The method of any one of claims 293-296, wherein the particles have less than about 10% change in charge variants of the therapeutic biologic after removing the first liquid, second liquid, and third liquid.

298. The method of any one of claims 293-297, wherein the particles have less than about 5% change in charge variants of the therapeutic biologic after removing the first liquid, second liquid, and third liquid.

299. The method of any one of claims 293-298, wherein the particles have less than about 3% change in charge variants of the therapeutic biologic after removing the first liquid, second liquid, and third liquid.

300. The method of any one of claims 293-299, wherein the particles have less than about 1% change in charge variants of the therapeutic biologic after removing the first liquid, second liquid, and third liquid.

301. The method of any one of claims 293-300, wherein the particles are substantially free from any change in charge variants of the therapeutic biologic after removing the first liquid, second liquid, and third liquid.

302. The method of any one of claims 159-301, wherein the particles have a surfactant content of less than about 10% by mass after removing the first liquid, second liquid, and third liquid.

303. The method of claim 302, wherein the particles have a surfactant content of less than about 5% by mass after removing the first liquid, second liquid, and third liquid.

304. The method of claim 302 or 303, wherein the particles have a surfactant content of less than about 3% by mass after removing the first liquid, second liquid, and third liquid.

305. The method of any one of claims 302-304, wherein the particles have a surfactant content of less than about 1% by mass after removing the first liquid, second liquid, and third liquid.

306. The method of any one of claims 302-305, wherein the particles have a surfactant content of less than about 0.1% by mass after removing the first liquid, second liquid, and third liquid.

307. The method of any one of claims 302-306, wherein the particles have a surfactant content of less than about 0.01% by mass after removing the first liquid, second liquid, and third liquid.

308. The method of any one of claims 302-307, wherein the particles have a surfactant content of less than about 0.001% by mass after removing the first liquid, second liquid, and third liquid.

309. The method of any one of claims 302-308, wherein the particles are substantially free from any surfactant content after removing the first liquid, second liquid, and third liquid.

310. The method of any one of claims 159-309, wherein the particles have less than about 3% of residual first liquid, second liquid, and third liquid by mass remaining after removing the first liquid, second liquid, and third liquid.

311. The method of claim 310, wherein the particles have less than about 2% of residual first liquid, second liquid, and third liquid by mass remaining after removing the first liquid, second liquid, and third liquid.

312. The method of claim 310 or 311, wherein the particles have less than about 1% of residual first liquid, second liquid, and third liquid by mass remaining after removing the first liquid, second liquid, and third liquid.

313. The method of any one of claims 310-312, wherein the particles have less than about 0.1% of residual first liquid, second liquid, and third liquid by mass remaining after removing the first liquid, second liquid, and third liquid.

314. The method of any one of claims 310-313, wherein the particles have less than about 0.01% of residual first liquid, second liquid, and third liquid by mass remaining after removing the first liquid, second liquid, and third liquid.

315. The method of any one of claims 310-314, wherein the particles are substantially free from any residual first liquid, second liquid, and third liquid by mass after removing the first liquid, second liquid, and third liquid.

316. The method of any one of claims 159-315, wherein the particles have less than about 5% of residual moisture by mass.

317. The method of claim 316, wherein the particles have less than about 3% of residual moisture by mass.

318. The method of claim 316 or 317, wherein the particles have less than about 2% of residual moisture by mass.

319. The method of any one of claims 316-318, wherein the particles have less than about 1% of residual moisture by mass.

320. The method of any one of claims 316-319, wherein the particles have less than about 0.1% of residual moisture by mass.

321. The method of any one of claims 316-320, wherein the particles have less than about 0.01% of residual moisture by mass.

322. The method of any one of claims 316-321, wherein the particles are substantially free from any residual moisture by mass.
